(19) United States
(12) Patent Application Publication
(10) Pub. No.: US 2014/0088187 A1
(43) Pub. Date: Mar. 27, 2014

(54) ANDROGEN RECEPTOR INHIBITORS AND METHODS OF USE THEREOF

(71) Applicants: Alan C. Rigby, Glen Ridge, NJ (US); Steven P. Balk, Needham, MA (US); Kumaran Shanmugasundaram, North Plainfield, NJ (US); Howard C. Shen, Pasadena, CA (US)

(72) Inventors: Alan C. Rigby, Glen Ridge, NJ (US); Steven P. Balk, Needham, MA (US); Kumaran Shanmugasundaram, North Plainfield, NJ (US); Howard C. Shen, Pasadena, CA (US)

(73) Assignee: BETH ISRAEL DEACONESS MEDICAL CENTER, Boston, MA (US)

(21) Appl. No.: 13/994,679

(22) PCT Filed: Dec. 14, 2011
(86) PCT No.: PCT/US2011/064852

§ 371 Date: Dec. 11, 2013

Related U.S. Application Data
(60) Provisional application No. 61/422,958, filed on Dec. 14, 2010.

(51) Int. Cl. C07D 317/58; C12Q 1/66

(52) U.S. Cl. CPC C07D 317/58; C12Q 1/66
USPC 514/465; 549/443; 435/375; 435/8

(57) ABSTRACT

The present invention relates to novel aryl sulfonamide compounds use of such compounds in the inhibition of androgen receptor and in the treatment of various diseases, disorders or conditions related to androgen receptor.

### RELATED APPLICATIONS

This application claims the benefit of U.S. Provisional Application 61/422,958, filed on Dec. 14, 2010. The entire contents of the above-referenced application are herein incorporated by reference.

### BACKGROUND OF THE INVENTION

The androgen receptor (AR) is a member of the steroid nuclear-receptor superfamily of ligand-dependent transcription factors. The binding of androgen to AR initiates the gene activation required for male sex development.

In oncology drug discovery, inhibitors (antagonists or partial antagonists) of androgen receptor function are useful for treatment of anti-androgen refractory prostate cancer.

Development of synthetic ligands that specifically bind to androgen receptors has been largely guided by trial and error method of drug design despite the importance of the androgen receptor in physiological processes and medical conditions such as prostate cancer and modulation of reproductive organ modulation. Previously, new ligands specific for androgen receptors were discovered in the absence of information on the three dimensional structure of the androgen receptor with a bound ligand. Before the present invention, researchers were essentially discovering androgen receptor ligands by probing in the dark and without the ability to visualize how the amino acids of the androgen receptor held a ligand in its grasp.

Prostate cancer is one of the most frequently diagnosed noncutaneous cancers among men in the United States. One of the approaches to the treatment of prostate cancer is by androgen deprivation. The male sex hormone, testosterone, stimulates the growth of cancerous prostatic cells and, therefore, is the primary fuel for the growth of prostate cancer. The goal of androgen deprivation is to decrease the stimulation by testosterone of the cancerous prostatic cells. Testosterone normally is produced by the testes in response to stimulation from a hormonal signal called luteinizing hormone (LH) which in turn is stimulated by luteinizing-hormone releasing hormone (LH-RH). Androgen deprivation is accomplished either surgically by bilateral orchidectomy or chemically by LH-RH agonists (LHRH) with or without nonsteroidal antiandrogens.

Current studies suggest that early androgen deprivation in patients with micrometastatic disease may indeed prolong survival (Messing E M, et al (1999), N Engl J Med 34, 1781-1788; Newling (2001), Urology 58(Suppl 2A), 50-55). Moreover, androgen deprivation is being employed in numerous new clinical settings, including neoadjuvant therapy prior to radical prostatectomy, long-term adjuvant therapy for patients at high risk for recurrence following radiation or surgery, neoadjuvant therapy for radiation, and treatment of biochemical recurrence following radiation or surgery (Carroll, et al (2001), Urology 58, 1-4; Horwitz E M, et al (2001), Int J Radiat Oncol Biol Phy March 15; 49(4), 947-56). Thus, more prostate cancer patients have become candidates for and are being treated by androgen ablation. Moreover, these prostate cancer patients are being treated earlier and longer than in the past, which in some cases may be as long as 10 or more years of androgen deprivation therapy.

Unfortunately, androgen deprivation therapy is fraught with significant side effects, including hot flashes, gynecomastia, osteoporosis, decreased lean muscle mass, depression and other mood changes, loss of libido, and erectile dysfunction (Stege R (2000), Prostate Suppl 10, 38-42). Consequently, complications of androgen blockade now contribute significantly to the morbidity, and in some cases the mortality, of men suffering from prostate cancer.

Given that more patients today are being treated by long-term androgen deprivation, osteoporosis has become a clinically important side effect in men suffering from prostate cancer undergoing androgen deprivation. Loss of bone mineral density (BMD) occurs in the majority of patients being treated by androgen deprivation by 6 months. New innovative approaches are urgently needed at both the basic science and clinical levels to decrease the incidence of androgen-deprivation induced osteoporosis in men suffering from prostate cancer.

The AR is comprised of an N-terminal transactivation domain, a DBD in the middle, and a C-terminal LBD to which androgen (testosterone or dihydrotestosterone (DHT)) binds, stimulating AR nuclear translocation and expression of multiple androgen regulated proteins such as prostate specific antigen (PSA). The standard treatment for metastatic PCa is suppression of testicular androgen production by surgical or medical castration (androgen deprivation therapy, ADT), but patients invariably relapse with a more aggressive form of PCa termed castration resistant PCa (CRPC). Significantly, the AR remains highly expressed in these CRPC tumors, and the expression of multiple AR target genes supports that AR transcriptional activity is restored. Mechanisms that may contribute to this restored AR activity include increased androgen synthesis by the tumor cells, AR gene amplification and other mechanisms that increase AR expression, AR somatic gain of-function mutations, AR alternative splicing that deletes the LBD, and activation of kinases that directly or indirectly enhance AR responses to low levels of androgen. A substantial proportion of CRPC patients respond to secondary therapies that further suppress androgen synthesis (ketoconazole, abiraterone). In contrast, a smaller fraction respond to established AR antagonists (flutamide, nilutamide, or bicalutamide) that compete with androgen for binding to the LBD, and these responses are generally modest and transient. Somatic AR mutations that markedly enhance the agonist activity of these antagonists are found in a subset of CRPC patients treated long-term with these drugs. However, it is important to note that the AR is wild-type in the majority of CRPC patients that relapse following ADT, and for reasons that remain unclear the current AR antagonists have limited effectiveness (Yuan and Balk, 2009).

MDV3100 is a recently developed AR antagonist that was selected for activity against PCa cells overexpressing a wild-type AR (Tran et al., 2009). In contrast to bicalutamide, which enhances AR nuclear translocation (Masiello et al., 2002), the MDV3100 liganded AR localizes primarily to the cytoplasm and does not have demonstrable agonist activity. Results of phase MI clinical trials indicate that it may be substantially more active than previous antagonists, although the majority of MDV3100 treated patients still relapse within one year with tumors that express high levels of PSA (indicative of AR reactivation). While the molecular basis for relapse and AR reactivation after MDV3100 is unclear, these observations support the potential efficacy of AR antagonists with novel MOAs in CRPC.

It is a goal of the invention to identify entirely novel AR antagonists that both prevent AR nuclear localization/chromatin binding and enhance AR degradation.

### SUMMARY OF THE INVENTION

In one aspect, the invention provides a compound comprising one or more functional groups that is capable of contacting or interacting with a binding pocket or a portion of a binding pocket of an AR ligand binding domain supporting or inducing the AR in an antagonized conformation, comprising any one or more of the following contacts:

(a) hydrogen bonding with Arg752 and Gln711;

(b) hydrogen bonding with a side chain amino group of Val746;

(c) hydrogen bonding with a side chain amide group of Gln783;

(d) hydrogen bonding with Asn705 and Gln783;

(e) hydrogen bonding with a side chain amine group of Asn705 of helix 3;

(f) interaction with a side chain amine group of Gln783 of helix 7;

(g) hydrogen bonding with residues Asn705 and Gln783;

(h) interaction with a carbonyl group of Gln783; and

(i) hydrophobic interaction with residues Leu707, Met745, and Met749 in a hydrophobic cleft.

In another aspect, the invention provides a compound of formula I,

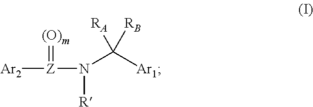

wherein,

Ar₁ is aryl or heteroaryl, each of which is optionally substituted;

Ar₂ is aryl or heteroaryl, each of which is optionally substituted;

R_{A} is H, alkyl, aralkyl, aryl, heteroaryl, halo, nitro, or CN, each of which is optionally substituted;

R_{B} is H, H, alkyl, aralkyl, aryl, heteroaryl, halo, nitro, or CN, each of which is optionally substituted;

R′ is H, alkyl, aryl, or aralkyl, each of which is optionally substituted;

Z is C or S, and

m 0, 1, or 2.

In one aspect, the invention provides a method for antagonizing activity of an androgen receptor, comprising contacting an androgen receptor with a compound of the invention.

In another aspect, the invention provides a method of antagonizing an androgen receptor, comprising administering an effective amount of the compound of the invention, to a subject.

In another aspect, the invention provides a method for treating a subject having a condition susceptible to treatment with an androgen receptor antagonist, comprising administering to the subject a therapeutically effective amount of a compound of the invention.

In another aspect, the invention provides a method for decreasing the activity and amount of androgen receptor in cells, comprising contacting the cells with a compound of the invention; wherein the amount of androgen receptors in the cells is decreased.

In another aspect, the invention provides a method of preventing or treating cancer in a subject, comprising administering an effective amount of the compound of the invention to a subject.

In another aspect, the invention provides a pharmaceutical composition comprising the compound of the invention, for use in treating a subject having a condition susceptible to treatment with an androgen receptor antagonist.

In another aspect, the invention provides a method for identifying a compound which antagonizes the activity of AR, the method comprising: contacting AR in an antagonized conformation with a compound under conditions suitable for antagonization of the activity of said AR; and detecting antagonization of the activity of said AR by the compound,

wherein the compound interacts with a binding site comprising one or more of

(a) hydrogen bonding with residues Arg752 and Gln711;

(b) hydrogen bonding with a side chain amino group of Val746;

(c) hydrogen bonding with a side chain amide group of Gln783;

(d) hydrogen bonding with Asn705 and Gln783;

(e) hydrogen bonding with a side chain amine group of Asn705 of helix 3;

(f) interaction with a side chain amine of Gln783 of helix 7;

(g) hydrogen bonding with residues Asn705 and Gln783;

(h) interaction with a carbonyl group of Gln783; and

(i) hydrophobic interaction with residues Leu707, Met745, and Met749 in a hydrophobic cleft.

In another aspect, the invention provides a method of identifying a compound that antagonizes AR, comprising:

a) using a three dimensional model/structure of a binding site of an antagonized conformation of AR, wherein said binding site comprises one or more of Arg752; Gln711; Val746; Gln783; Asn705; Leu707; Met745; and Met749; and

b) employing said antagonized conformation of AR template to select said AR antagonization compound, wherein said antagonziation compound binds to said binding site.

In another aspect, the invention provides a method of identifying a compound that antagonizes AR, the method comprising:

a. providing a three dimensional model/structure of a binding site of an antagonized conformation of AR, wherein said binding site comprises one or more of Arg752; Gln711; Val746; Gln783; Asn705; Leu707; Met745; and Met749; and
b. simulating a binding interaction between said binding site and a compound, wherein the interaction of the compound with the binding site occurs at one or more of Arg752; Gln711; Val746; Gln783; Asn705; Leu707; Met745; and Met749; and
c. determining whether said compound binds to one or more amino acid residues selected from the group consisting of, Arg752; Gln711; Val746; Gln783; Asn705; Leu707; Met745; and Met749, of said binding site, wherein said compound which binds to said amino acid residue of the binding site.

### BRIEF DESCRIPTION OF THE DRAWINGS

FIG. 1. Flow chart for the in-silico discovery of the Non-steroidal AR antagonist;

FIG. 2. The docked conformation of the series of Substituted N-(1,3-benzodioxol-5-yl methyl)benzene sulfonamide;

FIG. 3. Superimposition of crystal bicalutamide and the docked conformation of bicalutamide with RMSD of 0.555 A. The compound is show in the crystal structure complex (atomic colors) and the computationally docked pose of bicalutamide to validate the accuracy of the approach;

FIG. 4. The docked conformations of the 50 substituted N-(1,3-benzodioxol-5-yl methyl)benzene sulfonamide derivatives into the corepressor bound modeled AR;

FIG. 5. An in silico derived homology model of AR in an antagonist conformation;

FIG. 6. In silico identified compounds are competitive AR antagonists. Luciferase activity from COS7 cells transfected with AR and AR-responsive luciferase reporter plasmids followed by drug treatment for 24 hours. (a) Wildtype AR response to DHT (10 nM) alone or in the presence of bicalutamide (Bic, 10 μM) or compounds 1-57 (50 μM). Numbered arrows indicate the 16 compounds selected for further study. (b) Wildtype AR response to DHT (10 nM), Mifepristone (Mif, 100 nM), or the 16 selected compounds (50 μM). (c) AR N/C interaction stimulated by DHT (10 nM) alone or in the presence of Bic (10 μM) or chemotype A-F compounds (10 μM). (d) Wildtype AR response to increasing DHT concentrations (0-200 nM) in the presence of chemotype A-F compounds (10 μM). (e) T877A mutant AR response to DHT (10 nM), Bic (10 μM), hydroxyflutamide (HF, 100 nM) and chemotype A compounds (10 μM). (f) W741C mutant AR response to Bic (10 μM), HF (100 nM), and chemotype A compounds (10 μM). Error bars represent S.E.M.

FIG. 7. VP16 transactivation domain to the N-terminus of the full length AR (VP16-AR) to generate an AR with constitutive transcriptional activity;

FIG. 8. Decreased nuclear AR (at 10 μM), while further decreasing basal level PSA expression in steroid depleted medium;

FIG. 9. Compound A89, but not bicalutamide, stimulates a dose dependent decrease in AR protein as well as PSA levels in C4-2 cells in steroid depleted medium;

FIG. 10. Chemotype A compounds stimulate AR degradation. (a) AR and PSA protein levels in C4-2 cells following treatment with DHT (10 nM), A61, A89, MDV3100, or Bic for 24 hours. (b) AR and PSA protein levels in C4-2 cells following treatment with A89 in the presence of DHT (10 nM) for 24 hours. (c) AR protein levels in C4-2 cells following treatment with A89 (50 μM) alone or in the presence of MG132 proteasome inhibitor (MG, 10 μM). (d) AR protein levels in PC3-AR cells following treatment with cyclohexamide (CHX, 100 nM) and A89 (50 μM). (e) AR protein levels in VCaP cells following treatment with CHX (100 nM) and A89 (50 μM). (0 Luciferase activity from COS7 cells transfected with either AR WT or ARΔH12, VP16-NCoRc, and an AR-responsive luciferase reporter followed by treatment with Mif (100 nM) and A89 (50 μM) alone or in combination for 24 hours. Error bars represent S.E.M. (g) AR WT and ARΔH12 protein levels in transfected COS7 cells following treatment with A89 for 24 hours.

FIG. 11. Effects of linker modification (Generation 3 compounds) on AR expression and activity in C4-2 cells. C4-2 cells in CSS medium were treated for 16 hours with generation 3 compounds and blotted for AR and PSA expression.

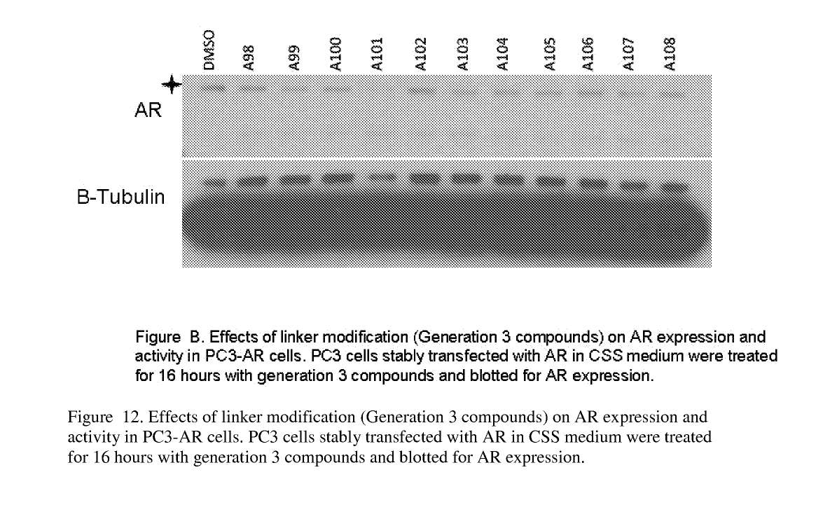

FIG. 12. Effects of linker modification (Generation 3 compounds) on AR expression and activity in PC3-AR cells. PC3 cells stably transfected with AR in CSS medium were treated for 16 hours with generation 3 compounds and blotted for AR expression.

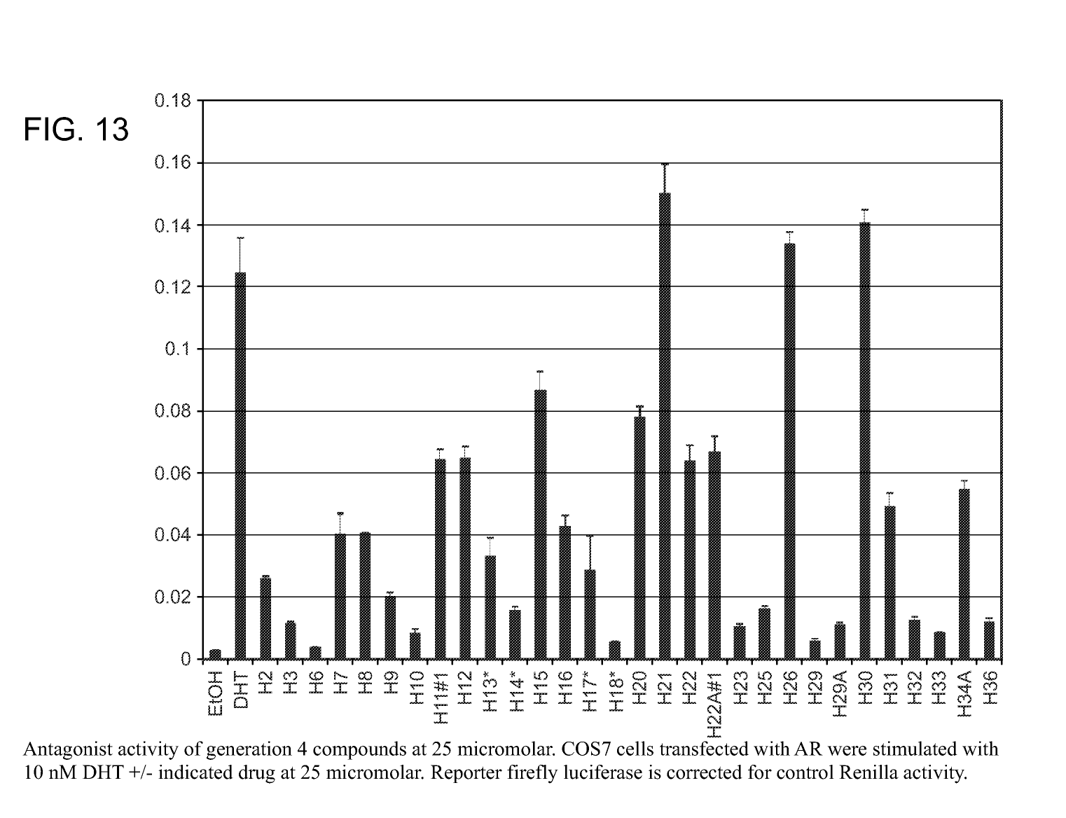

FIG. 13. Antagonist activity of generation 4 compounds at 25 micromolar. COS7 cells transfected with AR were stimulated with 10 nM DHT+/−indicated drug at 25 micromolar. Reporter firefly luciferase is corrected for control Renilla activity.

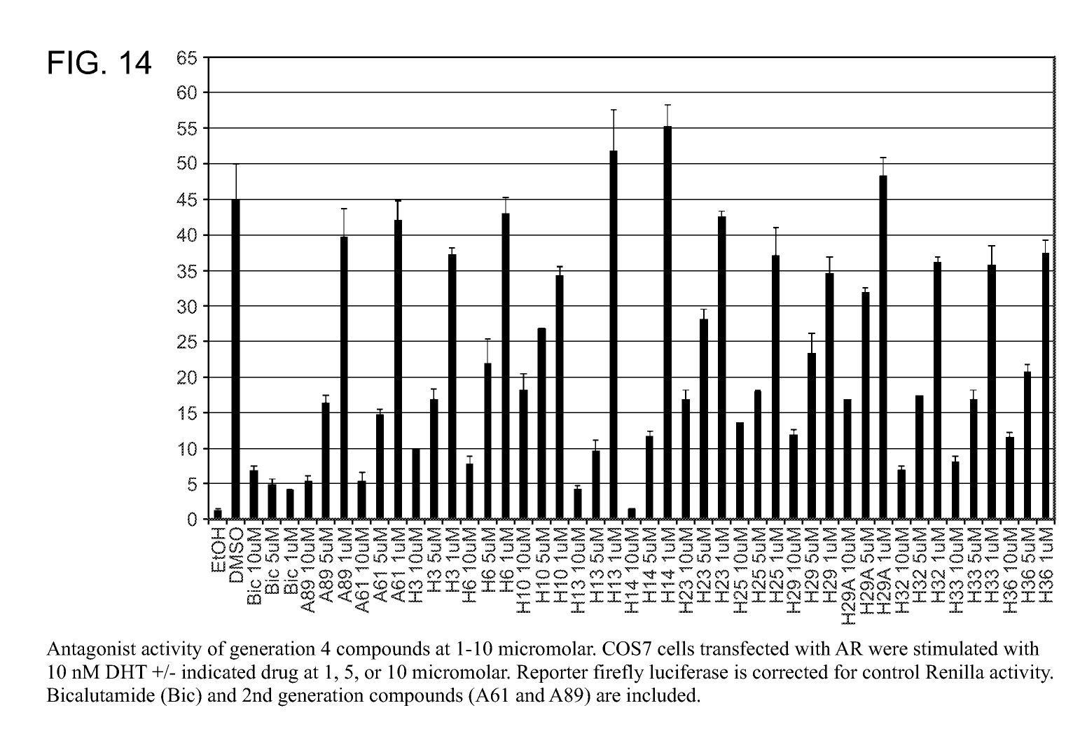

FIG. 14. Antagonist activity of generation 4 compounds at 1-10 micromolar. COS7 cells transfected with AR were stimulated with 10 nM DHT+/−indicated drug at 1, 5, or 10 micromolar. Reporter firefly luciferase is corrected for control Renilla activity. Bicalutamide (Bic) and 2nd generation compounds (A61 and A89) are included.

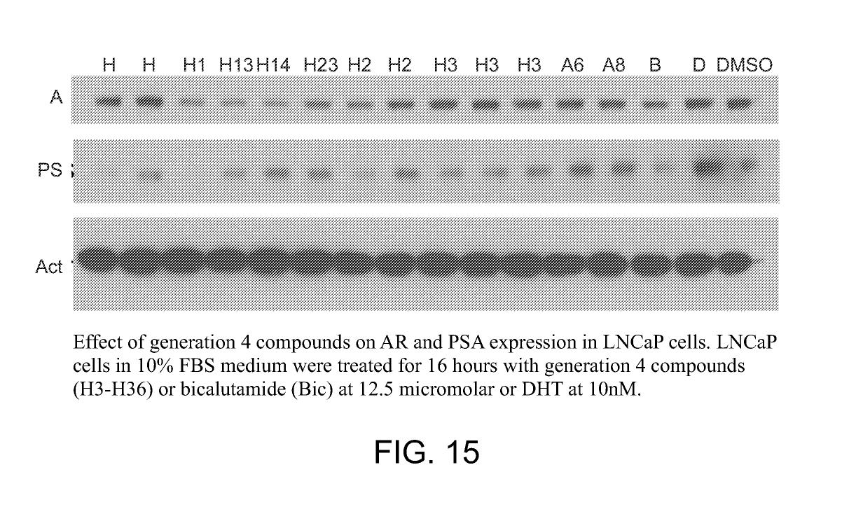

FIG. 15. Effect of generation 4 compounds on AR and PSA expression in LNCaP cells. LNCaP cells in 10% FBS medium were treated for 16 hours with generation 4 compounds (H3-H36) or bicalutamide (Bic) at 12.5 micromolar or DHT at 10 nM.

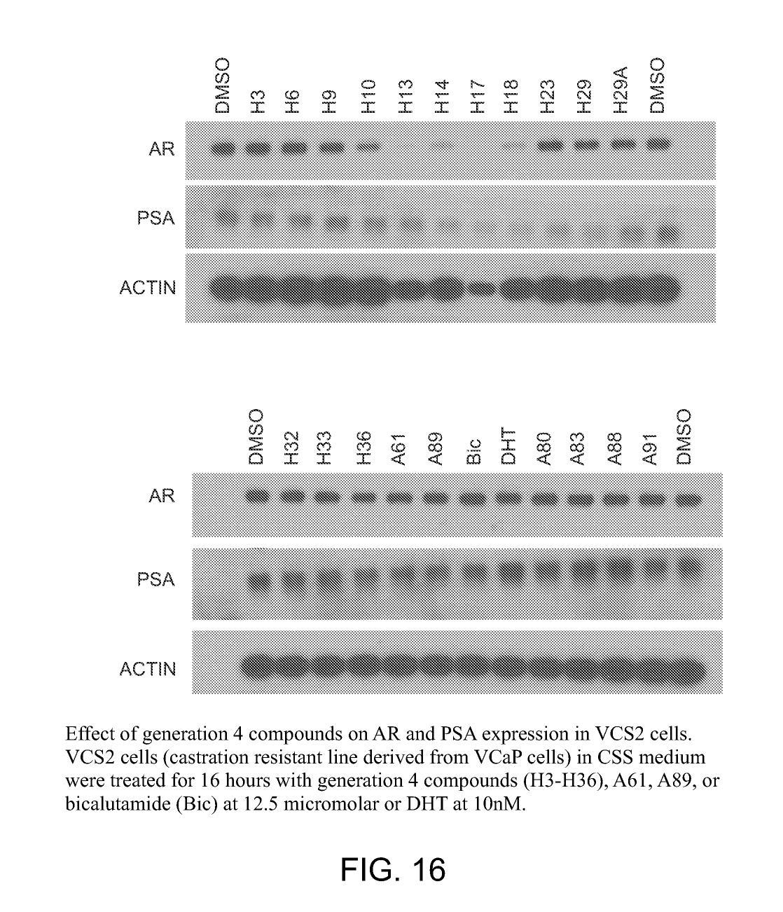

FIG. 16. Effect of generation 4 compounds on AR and PSA expression in VCS2 cells. VCS2 cells (castration resistant line derived from VCaP cells) in CSS medium were treated for 16 hours with generation 4 compounds (H3-H36), A61, A89, or bicalutamide (Bic) at 12.5 micromolar or DHT at 10 nM.

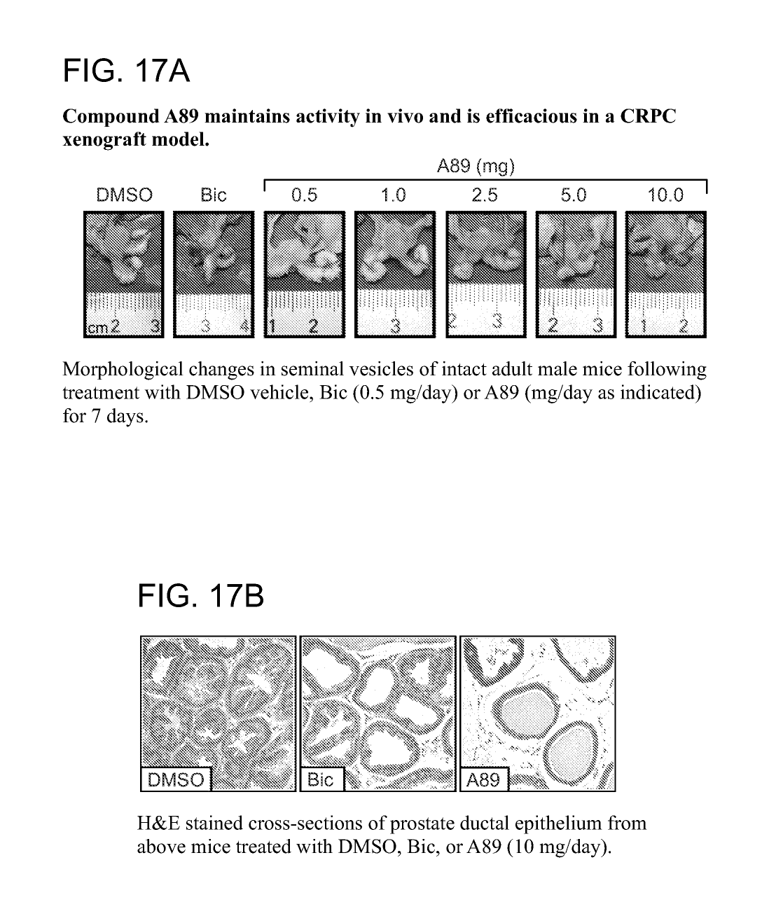

FIG. 17. Compound A89 maintains activity in vivo and is efficacious in a CRPC xenograft model. (a) Morphological changes in seminal vesicles of intact adult male mice following treatment with DMSO vehicle, Bic (0.5 mg/day) or A89 (mg/day as indicated) for 7 days. (b) H&E stained cross-sections of prostate ductal epithelium from above mice treated with DMSO, Bic, or A89 (10 mg/day). (c) AR and Ki-67 staining in pre- and post-castration relapsed VCaP xenograft tumor sections following treatment with A89 (5 mg/day) or Bic (0.5 mg/day) for 7 days. (d) mRNA levels of AR and AR-target genes in post-castration relapsed VCaP xenograft tumors following treatment with A89 (5 mg/day) for 7 days. Error bars represent S.E.M.

### DETAILED DESCRIPTION OF THE INVENTION

Compounds of the Invention

In one aspect, the invention provides a compound comprising one or more functional groups that is capable of contacting or interacting with a binding pocket or a portion of a binding pocket of an AR ligand binding domain supporting or inducing the AR in an antagonized conformation, comprising any one or more of the following contacts:

(a) hydrogen bonding with Arg752 and Gln711;

(b) hydrogen bonding with a side chain amino group of Val746;

(c) hydrogen bonding with a side chain amide group of Gln783;

(d) hydrogen bonding with Asn705 and Gln783;

(e) hydrogen bonding with a side chain amine group of Asn705 of helix 3;

(f) interaction with a side chain amine group of Gln783 of helix 7;

(g) hydrogen bonding with residues Asn705 and Gln783;

(h) interaction with a carbonyl group of Gln783; and

(i) hydrophobic interaction with residues Leu707, Met745, and Met749 in a hydrophobic cleft.

In another aspect, the invention provides a compound of formula I,

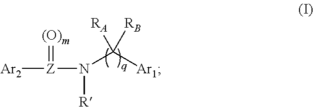

wherein,

Ar₁ is aryl or heteroaryl, each of which is optionally substituted;

Ar₂ is aryl or heteroaryl, each of which is optionally substituted;

R_{A} is H, alkyl, aralkyl, aryl, heteroaryl, halo, nitro, or CN, each of which is optionally substituted;

R_{B} is H, H, alkyl, aralkyl, aryl, heteroaryl, halo, nitro, or CN, each of which is optionally substituted;

R′ is H, alkyl, aryl, or aralkyl, each of which is optionally substituted;

Z is C or S,

q is 0 or 1; and

m 0, 1, or 2.

In certain embodiments, Ar1 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

In other embodiments, Ar2 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

In another embodiment, the invention provides a compound of formula I-A,

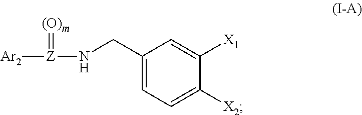

wherein,

X₁ and X₂, together with the atoms to which each is attached, form a fused carbocyclic, aryl, heterocyclic, or heteroaryl ring, each of which is optionally substituted; or

X₁ is halo or absent;

X₂ is halo or absent;

Ar₂ is aryl or heteroaryl, each of which is optionally substituted;

Z is C or S; and

m 0, 1, or 2.

In another embodiment, the invention provides a compound of formula II,

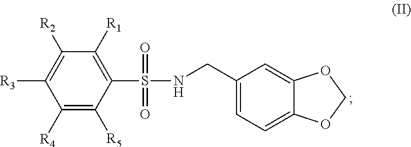

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and

each n is independently 0, 1, or 2.

In another embodiment, the invention provides a compound of formula II,

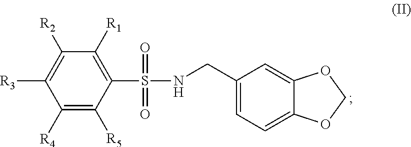

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and
each n is independently 0, 1, or 2;
   wherein the following compounds of formula H are excluded:

| Entry | R₁ | R₂ | R₃ | R₄ | R₅ |
|---|---|---|---|---|---|
| 1 | | | Cl | | |
| 2 | | | F | | |
| 3 | | CH₃ | | | CH₃ |
| 4 | | | Br | | |
| 5 | | Cl | | | |
| 6 | | Cl | F | | |
| 7 | | CH₃ | | | |
| 8 | | CH₃ | F | | |
| 9 | | | CH₃ | | |
| 10 | | CH₃ | Cl | CH₃ | |
| 11 | | CH₃ | CH₃ | | |
| 12 | Cl | Cl | | | |
| 13 | CH₃ | | Br | | |
| 14 | Cl | | Cl | | |
| 15 | Br | | Br | | |
| 16 | OCH₃ | Br | | | |
| 17 | | | OCH₃ | | |
| 18 | Cl | CH₃ | Cl | | |
| 19 | | | I | | |
| 20 | OCH₃ | Cl | Cl | | |
| 21 | Cl | | Cl | Cl | |
| 22 | | OCH₃ | Cl | | |
| 23 | Cl | Cl | Cl | | |
| 24 | Cl | Cl | OCH₃ | | |
| 25 | CH₃ | | CH₃ | OCH₃ | |
| 26 | Br | CH₃ | CH₃ | | |
| 27 | | | t-Bu | | |
| 28 | O—Et | Br | | | |
| 29 | F | | | | |
| 30 | | | C₆H₅CH₃ | | |
| 31 | | F | | | |
| 32 | Cl | | | | Cl |
| 33 | CH₃ | CH₃ | OCH₃ | | |
| 34 | | | F | OCH₃ | |
| 35 | | F | | | OCH₃ |
| 36 | Cl | Cl | OCH₃ | | |
| 37 | Br | OCH₃ | | | |
| 38 | | Cl | OCH₃ | | |
| 39 | | I | OCH₃ | | |
| 40 | | | | SCH₃ | |
| 41 | O—Et | Cl | | | |
| 42 | Cl | Cl | O—Et | | |
| 43 | | | F | O—Et | |
| 44 | CH₃ | CH₃ | O—Et | | |
| 45 | | OCH₃ | Br | | |
| 46 | CH₃ | Cl | Cl | | |
| 47 | | CH₃ | O—Et | | |
| 48 | | | Et | | |
| 49 | | F | F | | |

In a further embodiment, R₁ and R₅ are each independently H, halo, or optionally substituted alkyl;

R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′;

R₃ is

(a) H, alkyl, or haloalkyl; (b) halogen, nitro, or cyano; or (c) C(O)R′, C(O)OR′, OR′, NR″R′, NR″C(O)R′, or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and
each n is independently 0, 1, or 2.

In another embodiment, R₃ is haloalkyl.

In certain embodiments, R₂ and R₄ are each independently H or haloalkyl.

In various embodiments, R₃ is OR′ and R′ is haloalkyl.

In other embodiments, R₂ and R₄ are each independently H or OR′ and R′ is haloalkyl.

In still other embodiments, R₃ is NR″R′, NR″C(O)R′, or NR″S(O)ₙR′.

In another embodiment, R₃ and R₄ are each independently H, CH₃, CF₃, CN, COCH₃, COOH, COCH₂CH₃, COCH₂CH₂CH₃, CONH₂, NO₂, NC, NHCOCH₃, NHCOCH₂CH₃, NHCOCH₂Br, NHCOCH₂Cl, N(COCH₃)₂, N(COCH₂CH₃)₂, NHCOCF₃, NHSO₂CH₃, NHCOCH₂C(CH₃)₃, NCS, OCF₃, SCN, or SO₂CH₃.

In one embodiment, the invention provides a compound of formula III:

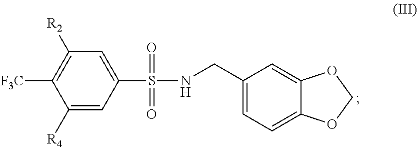

wherein R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′.

In certain embodiments, R₂ is H and R₄ is nitro, —NC, —S—CN, —N═C═S, or cyano.

In another embodiment, the invention provides a compound of formula IV:

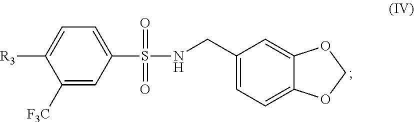

wherein R₃ is (a) H, alkyl, or haloalkyl; (b) halogen, nitro, —NC, or cyano; or (c) C(O)R′, C(O)OR′, OR′, NR″R′, NR″C(O)R′, or NR″S(O)ₙR′.

In certain embodiments, R₃ is nitro, —NC, cyano, C(O)R′, C(O)OR′, or NR″R′, wherein

each R′ is independently H, alkyl, or alkynyl, each of which is optionally substituted; and
each R″ is independently H or optionally substituted alkyl.

In another embodiment, the invention provides a compound of formula V:

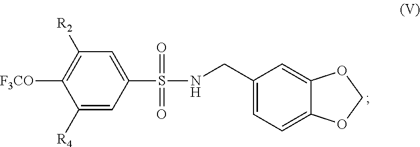

wherein R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′;

In a further embodiment, R₂ is H and R₄ is C(O)R′, C(O)N(R″)(R′), or S(O)ₙR′; and n is 2; wherein

each R′ is independently H, alkyl, cycloalkyl, heterocycloalkyl, aryl, heteroaryl, aralkyl, alkenyl, alkynyl, haloalkyl, or alkoxy, each of which is optionally substituted; and

each R″ is independently H or optionally substituted alkyl.

In another embodiment, the invention provides a compound of formula VI:

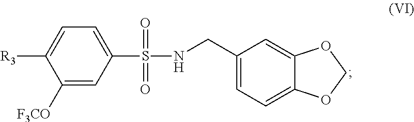

wherein R₃ is (a) H, alkyl, or haloalkyl; (b) halogen, nitro, —NC, or cyano; or (c) C(O)R′, C(O)OR′, OR′, NR″R′, NR″C(O)R′, or NR″S(O)ₙR′.

In one embodiment, R₃ is nitro, C(O)R′, or C(O)OR′, wherein each R′ is independently H, alkyl, or alkynyl, each of which is optionally substituted.

Representative compounds of the invention include, but are not limited to, the following compounds of Table 1 below.

TABLE 1

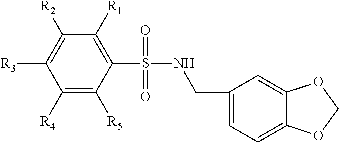

| Entry | R₁ | R₂ | R₃ | R₄ | R₅ |
|---|---|---|---|---|---|
| 1 | | —NO₂ | —CF₃ | | |
| 2 | | —NC | —CF₃ | | |
| 3 | | —SCN | —CF₃ | | |
| 4 | | —N═C═S | —CF₃ | | |
| 5 | | —CN | —CF₃ | | |
| 6 | | —CF₃ | —NO₂ | | |
| 7 | | —CF₃ | 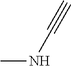 | | |
| 8 | | —CF₃ | —NC | | |
| 9 | | —CF₃ | —COOH | | |
| 10 | | —CF₃ | —C(O)CH₂CH₃ | | |
| 11 | | —CF₃ | —C(O)(CH₂)₂CH₃ | | |
| 12 | | —CF₃ | —C(O)CH₃ | | |
| 13 | | —SO₂CH₃ | —OCF₃ | | |
| 14 | | —C(O)CH₃ | —OCF₃ | | |
| 15 | | —C(O)NH₂ | —OCF₃ | | |
| 16 | | —OCF₃ | —C(O)CH₃ | | |
| 17 | | —OCF₃ | —C(O)CH₂CH₃ | | |
| 18 | | —OCF₃ | —COOH | | |
| 19 | | —OCF₃ | —NO₂ | | |
| 20 | | | —N(COCH₃)₂ | | |
| 21 | | | —NHCOCH₃ | | |
| 22 | | | —NHCOCH₂CH₃ | | |
| 23 | | | —NHCOCH₂Br | | |
| 24 | | | —NHCOCH₂Cl | | |
| 25 | | | 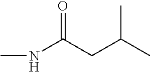 | | |
| 26 | | | —NHSO₂CH₃ | | |
| 27 | | | —NHCOCF₃ | | |
| 28 | | | —N(COCH₂CH₃)₂ | | |
| 29 | | —NHCOCH₂Cl | | | |
| 30 | | —NHCOCH₂Br | | | |
| 31 | | —SCN | | | |
| 32 | | —C(O)CH₂CH₃ | —CH₃ | | |
| 33 | | —C(O)CH₃ | —CH₃ | | |
| 34 | | —CF₃ | —CN | | |
| 35 | | —C(O)CH₃ | —NO₂ | | |
| 36 | | —C(O)CH₂CH₃ | —NO₂ | | |
| 37 | | | 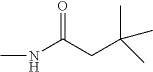 | | |
| 38 | | —C(O)CH₂CH₃ | —OCF₃ | | |

In another aspect, the invention provides a compound of formula VII,

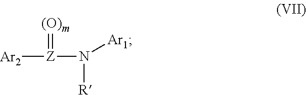

wherein,

Ar₁ is aryl or heteroaryl, each of which is optionally substituted;

Ar₂ is aryl or heteroaryl, each of which is optionally substituted;

R′ is H, alkyl, aryl, or aralkyl, each of which is optionally substituted;

Z is C or S, and

m 0, 1, or 2.

In certain embodiments, Ar1 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

In other embodiments, Ar2 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

In another embodiment, the invention provides a compound of formula VII-A,

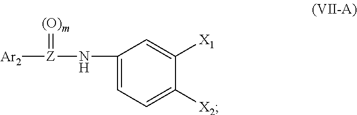

wherein,

X₁ and X₂, together with the atoms to which each is attached, form a fused carbocyclic, aryl, heterocyclic, or heteroaryl ring, each of which is optionally substituted; or

X₁ is halo or absent;

X₂ is halo or absent;

Ar₂ is aryl or heteroaryl, each of which is optionally substituted;

Z is C or S; and

m 0, 1, or 2.

In another embodiment, the invention provides a compound of formula VIII,

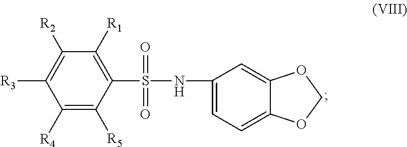

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and

each n is independently 0, 1, or 2.

In certain embodiments, the invention provides a compound selected from the following:

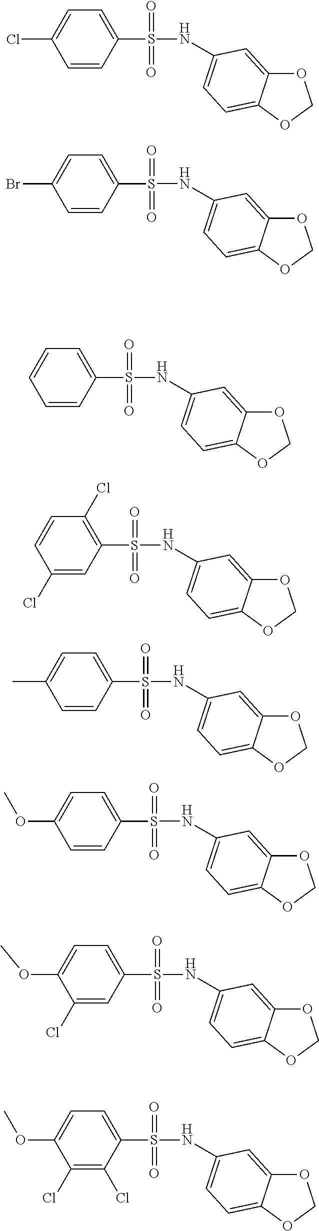

In another aspect, the invention provides a compound of formula IX,

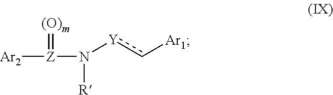

wherein,

Ar₁ is aryl or heteroaryl, each of which is optionally substituted;

Ar₂ is aryl or heteroaryl, each of which is optionally substituted;

R′ is H, alkyl, aryl, or aralkyl, each of which is optionally substituted;

Z is C or S,

Y is S(O). or N; and

each m independently 0, 1, or 2.

In certain embodiments, Ar1 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

In other embodiments, Ar2 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

In another embodiment, the invention provides a compound of formula IX-A,

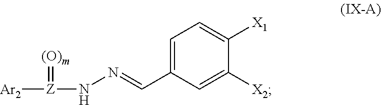

wherein,

X₁ and X₂, together with the atoms to which each is attached, form a fused carbocyclic, aryl, heterocyclic, or heteroaryl ring, each of which is optionally substituted; or

X₁ is halo or absent;

X₂ is halo or absent;

Ar₂ is aryl or heteroaryl, each of which is optionally substituted;

Z is C or S; and

m 0, 1, or 2.

In another embodiment, the invention provides a compound of formula X,

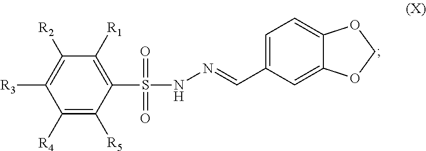

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and

each n is independently 0, 1, or 2.

In certain embodiments, the invention provides a compound selected from the following:

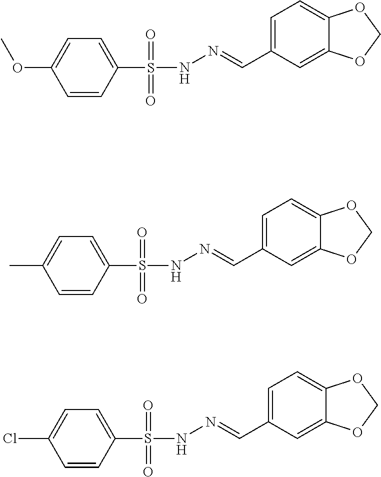 [SMILES: COc1ccc(S(=O)(=O)N/N=C/c2ccc3c(c2)OCO3)cc1 ; Cc1ccc(S(=O)(=O)N/N=C/c2ccc3c(c2)OCO3)cc1 ; O=S(=O)(N/N=C/c1ccc2c(c1)OCO2)c1ccc(Cl)cc1]

In another embodiment, the invention provides a compound as described herein, wherein the compound is a selective androgen receptor antagonist.

In certain embodiments, the selective androgen receptor antagonist is a selective androgen receptor partial antagonist.

In a further embodiment, the compound of the invention is a selective androgen receptor reducing compound.

In a further embodiment, the compound of the invention is a selective androgen receptor degrading compound.

In certain embodiments, the compound of the invention reduces AR cytoplasmic-nuclear translocation.

In other embodiments, the compound of the invention degrades AR.

A compound of the present invention which functions as an “antagonist” of the androgen receptor can bind to the androgen receptor and block or inhibit the androgen-associated responses normally induced by a natural androgen receptor ligand.

The invention also provides for a pharmaceutical composition comprising a compound of formula I, or a pharmaceutically acceptable ester, salt, or prodrug thereof, together with a pharmaceutically acceptable carrier.

Another object of the present invention is the use of a compound as described herein (e.g., of any formulae herein) in the manufacture of a medicament for use in the treatment of a disorder or disease herein. Another object of the present invention is the use of a compound as described herein (e.g., of any formulae herein) for use in the treatment of a disorder or disease herein.

In another aspect, the invention provides a method of method of synthesizing a compound of formula I. The synthesis of the compounds of the invention can be found in the Examples below.

Another embodiment is a method of making a compound of any of the formulae herein using any one, or combination of, reactions delineated herein. The method can include the use of one or more intermediates or chemical reagents delineated herein.

Another aspect is an isotopically labeled compound of any of the formulae delineated herein. Such compounds have one or more isotope atoms which may or may not be radioactive (e.g., ³H, ²H, ¹⁴C, ¹³C, ³⁵S, ³²P, ¹²⁵I, and ¹³¹I) introduced into the compound. Such compounds are useful for drug metabolism studies and diagnostics, as well as therapeutic applications.

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999, and subsequent editions thereof.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxan, tetrahydrofuran or methanol.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included in the present invention. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds of this invention may also be represented in multiple tautomeric forms, in such instances, the invention expressly includes all tautomeric forms of the compounds described herein. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present invention. All crystal forms of the compounds described herein are expressly included in the present invention.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Methods of the Invention

In one aspect, the invention provides a method for modulating activity of an androgen receptor, comprising contacting an androgen receptor with a compound disclosed in the invention.

In one embodiment, the androgen receptor is in a cell.

In another aspect, the invention provides a method of antagonizing an androgen receptor, comprising administering an effective amount of the compound disclosed in the invention, to a subject.

In a further embodiment, the compound reduces AR cytoplasmic-nuclear translocation or degrades AR.

In another aspect, the invention provides a method for treating a subject having a condition susceptible to treatment with an androgen receptor antagonist, comprising administering to the subject a therapeutically effective amount of a compound disclosed in the invention.

In certain embodiments, the condition is selected from: cancer, chronic fatigue syndrome; chronic myalgia; acute fatigue syndrome and muscle loss; complicated fractures; periodontal disease; wasting secondary to fractures and wasting in connection with chronic obstructive pulmonary disease, wasting in connection with chronic liver disease, wasting in connection with AIDS, cancer cachexia, cardiomyopathy; thrombocytopenia; growth retardation in connection with Crohn's disease; short bowel syndrome; irritable bowel syndrome; inflammatory bowel disease; Crohn's disease and ulcerative colitis; complications associated with transplantation; obesity and growth retardation associated with obesity; anorexia; hypercortisolism and Cushing's syndrome; Paget's disease; osteoarthritis; osteochondrodysplasias; depression, nervousness, irritability and stress; cardiac dysfunction; cachexia and protein loss due to chronic illness; hyperinsulinemia; wasting in connection with multiple sclerosis, Kennedy's disease or other neurodegenerative disorders; hypothermia; congestive heart failure; lipodystrophy; muscular atrophy; musculoskeletal impairment; sleep disorders; hirsutism, acne, seborrhea, androgenic alopecia, anemia, hyperpilosity, benign prostate hypertrophy, adenomas and neoplasies of the prostate and malignant tumor cells expressing the androgen receptor; osteosarcoma; hypercalcemia of malignancy; metastatic bone disease; endometriosis and polycystic ovary syndrome; eclampsia of pregnancy and preterm labor; hypogonadism, male and female sexual dysfunction, hair loss, and Reaven's Syndrome.

In certain embodiments, the subject has cancer.

In a further embodiment, the subject has androgen dependent prostate cancer.

In another embodiment, the subject has androgen independent prostate cancer.

In still other embodiments, the subject has prostate cancer that has relapsed after AR deprivation therapy (ADT), which is referred to as castraction resistant prostate cancer, or the subject has a condition that may progress to prostate cancer including prostate intraepithelial neoplasi (PIN) or prostatic inflammatory atrophy (PIA).

In another embodiment, the invention provides a method further comprising the step of identifying a subject having a condition susceptible to treatment with the compound disclosed in the invention.

In another aspect, the invention provides a method for decreasing the activity and amount of androgen receptor in cells, comprising contacting the cells with a compound disclosed in the invention; wherein the amount of androgen receptors in the cells is decreased.

In one embodiment, the decreasing activity and amount of androgen receptor in cells comprises one or more of:

(a) preventing agonist binding and activation of AR;

(b) preventing nuclear access of the AR;

(c) preventing chromatin binding of the AR;

(d) decreasing AR protein or message levels in the cells; or

(e) degrading the AR.

In another aspect, the invention provides a method of preventing or treating cancer in a subject, comprising administering an effective amount of the compound disclosed in the invention to a subject.

In one embodiment, the invention provides a method further comprising the use of an additional therapeutic agent.

In certain embodiments, the additional therapeutic agent is an anti-cancer drug.

In a further embodiment, the additional therapeutic is an AR drug.

In various embodiments, the invention includes any of the methods as described above wherein the subject is a human.

In another aspect, the invention provides a pharmaceutical composition comprising the compound disclosed in the invention, for use in treating a subject having a condition susceptible to treatment with an androgen receptor antagonist.

In another aspect, the invention provides a method for identifying a compound which modulates the activity of AR, the method comprising: contacting AR in an antagonized conformation with a compound under conditions suitable for modulation of the activity of said AR; and detecting modulation of the activity of said AR by the compound,

wherein the compound interacts with a binding site comprising one or more of

(a) hydrogen bonding with residues Arg752 and Gln711;

(b) hydrogen bonding with a side chain amino group of Val746;

(c) hydrogen bonding with a side chain amide group of Gln783;

(d) hydrogen bonding with Asn705 and Gln783;

(e) hydrogen bonding with a side chain amine group of Asn705 of helix 3;

(f) interaction with a side chain amine of Gln783 of helix 7;

(g) hydrogen bonding with residues Asn705 and Gln783;

(h) interaction with a carbonyl group of Gln783; and

(i) hydrophobic interaction with residues Leu707, Met745, and Met749 in a hydrophobic cleft.

In another aspect, the invention provides a method of identifying a compound that modulates AR, comprising:

a) using a three dimensional model/structure of a binding site of an antagonized conformation of AR, wherein said binding site comprises one or more of Arg752; Gln711; Val746; Gln783; Asn705; Leu707; Met745; and Met749; and

b) employing said antagonized conformation of AR template to select said AR modulator compound, wherein said modulator compound binds to said binding site.

In another aspect, the invention provides a method of identifying a compound that modulates AR, the method comprising:

c. providing a three dimensional model/structure of a binding site of an antagonized conformation of AR, wherein said binding site comprises one or more of Arg752; Gln711; Val746; Gln783; Asn705; Leu707; Met745; and Met749; and
d. simulating a binding interaction between said binding site and a compound, wherein the interaction of the compound with the binding site occurs at one or more of Arg752; Gln711; Val746; Gln783; Asn705; Leu707; Met745; and Met749; and
c. determining whether said compound binds to one or more amino acid residues selected from the group consisting of, Arg752; Gln711; Val746; Gln783; Asn705; Leu707; Met745; and Met749, of said binding site, wherein said compound which binds to said amino acid residue of the binding site.

In one aspect, compounds disclosed in the present invention may be useful to block or inhibit (“antagonize”) the function of the androgen receptor in the prostate of a male individual or in the uterus of a female individual. The antagonism of the AR in the prostate can be assayed through observation of minimal effects on prostate growth in castrated rodents and antagonism of prostate growth induced by AR agonists.

The compounds disclosed in the present invention can be used in the treatment of prostate cancer, either alone or as an adjunct to GnRH agonist/antagonist therapy, for their ability to restore bone, or as a replacement for antiandrogen therapy because of their ability to antagonize androgen in the prostate, and minimize bone depletion. Further, the compounds of the present invention can be used for their ability to restore bone in the treatment of pancreatic cancer as an adjunct to treatment with antiandrogen, or as monotherapy for their antiandrogenic properties, offering the advantage over traditional antiandrogens of being bone-sparing. Additionally, compounds of this invention can increase the number of blood cells, such as red blood cells and platelets, and can be useful for the treatment of hematopoietic disorders, such as aplastic anemia. Thus, considering their tissue selective androgen receptor agonism listed above, the compounds of this invention are ideal for hormone replacement therapy in hypogonadic (androgen deficient) men.

Representative compounds disclosed in the present invention typically display submicromolar binding affinity for the androgen receptor. Compounds of this invention are therefore useful in treating mammals suffering from disorders related to androgen receptor function. Pharmacologically effective amounts of the compound, including the pharmaceutically effective salts thereof, are administered to the mammal, to treat disorders related to androgen receptor function.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

Thus, in another aspect of the invention, methods for the treatment of cancer are provided comprising administering a therapeutically effective amount of an inventive compound (i.e., of any of the formulae herein), as described herein, to a subject in need thereof. In certain embodiments, the subject is identified as in need of such treatment. In certain embodiments, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells,” as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like.

For any of the methods described above, a compound disclosed in the invention may be used. Compounds disclosed in the invention used in the methods of the invention include those specifically delineated as individual species, as well as compounds encompassed by the following formulae:

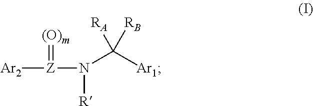

wherein,

Ar₁ is aryl or heteroaryl, each of which is optionally substituted;

Ar₂ is aryl or heteroaryl, each of which is optionally substituted;

R_{A} is H, alkyl, aralkyl, aryl, heteroaryl, halo, nitro, or CN, each of which is optionally substituted;

R_{B} is H, H, alkyl, aralkyl, aryl, heteroaryl, halo, nitro, or CN, each of which is optionally substituted;

R′ is H, alkyl, aryl, or aralkyl, each of which is optionally substituted;

Z is C or S, and

m 0, 1, or 2.

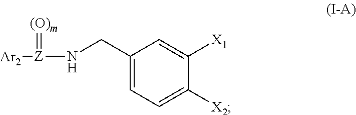

wherein,

X₁ and X₂, together with the atoms to which each is attached, form a fused carbocyclic, aryl, heterocyclic, or heteroaryl ring, each of which is optionally substituted; or

X₁ is halo or absent;

X₂ is halo or absent;

Ar₂ is aryl or heteroaryl, each of which is optionally substituted;

Z is C or S; and

m 0, 1, or 2.

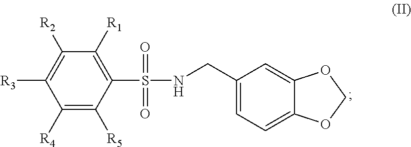

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR % S(O)ₙC(O)R % S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and
each n is independently 0, 1, or 2.

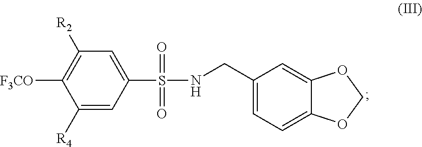

wherein R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′.

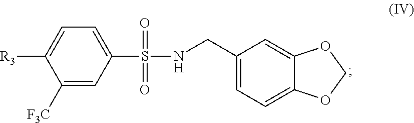

wherein R₃ is (a) H, alkyl, or haloalkyl; (b) halogen, nitro, —NC, or cyano; or (c) C(O)R′, C(O)OR′, OR′, NR″R′, NR″C(O)R′, or NR″S(O)ₙR′.

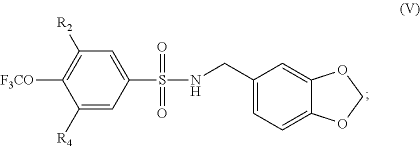

wherein R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′;

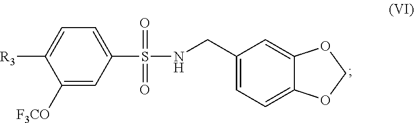

wherein R₃ is (a) H, alkyl, or haloalkyl; (b) halogen, nitro, —NC, or cyano; or (c) C(O)R′, C(O)OR′, OR′, NR″R′, NR″C(O)R′, or NR″S(O)ₙR′.

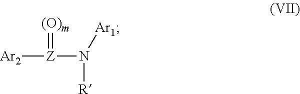

wherein,

Ar₁ is aryl or heteroaryl, each of which is optionally substituted;

Ar₂ is aryl or heteroaryl, each of which is optionally substituted;

R′ is H, alkyl, aryl, or aralkyl, each of which is optionally substituted;

Z is C or S, and

m 0, 1, or 2.

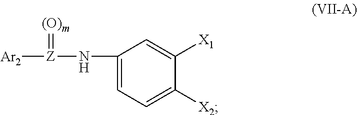

wherein,

X₁ and X₂, together with the atoms to which each is attached, form a fused carbocyclic, aryl, heterocyclic, or heteroaryl ring, each of which is optionally substituted; or

X₁ is halo or absent;

X₂ is halo or absent;

Ar₂ is aryl or heteroaryl, each of which is optionally substituted;

Z is C or S; and

m 0, 1, or 2.

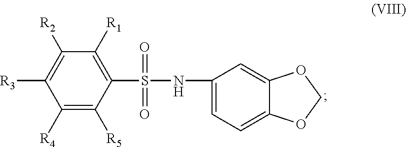

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and

each n is independently 0, 1, or 2.

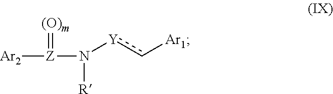

wherein,

Ar₁ is aryl or heteroaryl, each of which is optionally substituted;

Ar₂ is aryl or heteroaryl, each of which is optionally substituted;

R′ is H, alkyl, aryl, or aralkyl, each of which is optionally substituted;

Z is C or S,

Y is S(O)ₘ or N; and

each m independently 0, 1, or 2.

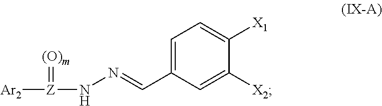

wherein,

X₁ and X₂, together with the atoms to which each is attached, form a fused carbocyclic, aryl, heterocyclic, or heteroaryl ring, each of which is optionally substituted; or

X₁ is halo or absent;

X₂ is halo or absent;

Ar₂ is aryl or heteroaryl, each of which is optionally substituted;

Z is C or S; and

m 0, 1, or 2.

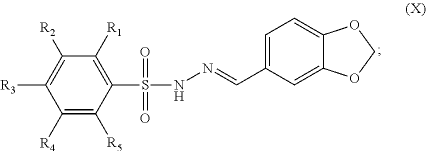

wherein,

R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;

R₂ and R₄ are each independently

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;

R₃ is

(a) H, alkyl, cycloalkyl, heterocycloalkyl, aryl, heteroaryl, aralkyl, alkenyl, alkynyl, haloalkyl, or alkoxy, each of which is optionally substituted;

(b) halogen, nitro, cyano, —NC, —S—CN, or —N═C═S; or

(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and

each n is independently 0, 1, or 2.

In certain embodiments, the invention provides a method of treatment of any of the disorders described herein, wherein the subject is a human.

In accordance with the foregoing, the present invention further provides a method for preventing or treating any of the diseases or disorders described above in a subject in need of such treatment, which method comprises administering to said subject a therapeutically effective amount of a compound of the invention or a pharmaceutically acceptable salt thereof. For any of the above uses, the required dosage will vary depending on the mode of administration, the particular condition to be treated and the effect desired.

AR Homology Modeling

It is well established that ligand binding causes movement of helix 12 within the ligand-binding domain (LBD) of the nuclear receptor superfamily of transcription factors with movement towards helices 3-5, which stabilizes ligand binding and generates a hydrophobic cleft for th subsequent binding of coactivator proteins via leucine-x-x-leucine-leucine (LxxLL) motifs in the NTD (aminoacids 23-27, FQNLF). Agonist bound AR undergoes dissociation from chaperones, dimerization, phosphorylation, translocation to the nucleus, and binding to the androgen response elements (ARE). The transactivation of AR-regulated gene expression is ensured by further recruitment of coactivators such as tartrate-resistant acid phosphatase220 (TRAP220) and steroid receptor coactivators (SRC). AR antagonist like bicalutamide, flutamide, and nilutamide currently used for treatment of PCa compete with androgens or AR agonists for the ligand-binding pocket and it is believed function by displacing helix 12, thus preventing the formation of coactivator-binding cleft preventing the coactivator recruitment. However, a paucity of the AR LBD in an antagonized structure has precluded definitive studies supporting this and other hypotheses. This unique antagonist induced conformation of AR enhances the recruitment of corepressor proteins like nuclear receptor corepressor (NCoR), silencing mediator for retinoic acid and thyroid hormone receptors (SMRT) with the extended LxxLL-like motifs interacting with the helices 3, 4, 5 thereby down regulating the over expression of AR modulated genes in prostate cancer.

To overcome some of these structural limitations a homolog model of the AR LBD has been generated in what is believed to be the AR antagonized conformation using the comparative protein modeling suite ORCHESTRAR. To do so the structural details of the corepressor NCoR bound PR (PDB code: 2OVM) has been used, as well as a wealth of structural data of the AR in an agonized conformation as template structures. The percent sequence identity of 52% was observed for the alignment of the NCoR bound PR with the wild type AR (PDB code: 2AM9). The sequence alignment had the homolog score of 276.90 and significance score of 36.3 which were used to model the conserved regions using the ORCHESTRAR. After searching the loops and fixing the side chains, the obtained structure was minimized with MMFF94 forcefield using the biopolymer module. The AR model was submitted to PROCHECK and the quality of the protein structure evaluated by Ramachandran plot. The modeled AR structure in the antagonized conformation was superimposed with wild type AR structure and identified a RMSD of 3.627 A.

GALAHAD Pharmacophore Model and 3D Pharmacophore Triplet Hypothesis:

Ligand-based computational screens were carried out using a highly refined training set of 23 active/reference compounds that were subjected to the pharmacophore alignment using the GALAHAD. The molecules were aligned using the pharmacophore features and the best single conformer pharmacophore model (out of the ˜250 that were generated) was selected to represent the chemical descriptors responsible for antagonist activity using the triplet tuplets. Pharmacophore triplet tuplets were generated using the pharmacophore feature like positive nitrogens, negative centers, hydrogen bond donors, acceptors atoms; and hydrophobic center with the multiple edge lengths binned at 0.5 A intervals.

Several databases that are within our in house library of 1.3 M compounds were screened. This library consists of compounds from: Chembridge, Asinex, NCI and others that are minimized using the CONCORD and subjected to the tuplet conformer generation. A 3D pharmacophoric tuplet hypothesis that was generated with the 100 random conformations of the active molecules from our GALAHAD model was used to screen the database of compounds. The above similar procedure used for the one of the identified lead scaffold N-(1,3-benzodioxol-5ylmethy)benzenesulfonamide.

Importantly all ligands were energy minimized using a standard Tripos force field that employs Powell minimization and simplex optimization with a distance dependent dielectric function and an energy gradient of 0.01 kcal/mol\acute{Å} with maximum of 1000 iterations. The ligands identified using our ligand-based pharmacophore were then docked into our homolog modeled AR conformation. This structure-based computational docking was initially carried out using the Surflex-dock with the initial generation of the protomol possessing a threshold range of 0.50 A by selecting the crucial interacting residues 705ASN, 711GLN, 752ARG and 877THR. All compounds that achieved a superior ranking to the known actives that were doped into our in house library were evaluated by using the SARCHITECT ADMETOX models. These ADME models: absorption rate constant, blood brain barrier penetration, bioavailabilty, elimination rate constant and protein binding were used as a filter to triage compounds that would likely fail later stage development on the basis of these metrics. The evaluated toxicity models are carinogenicity in rat and mouse models, mutagenicity, and HERO toxicity were also used to filter our best docked and ranked compounds.

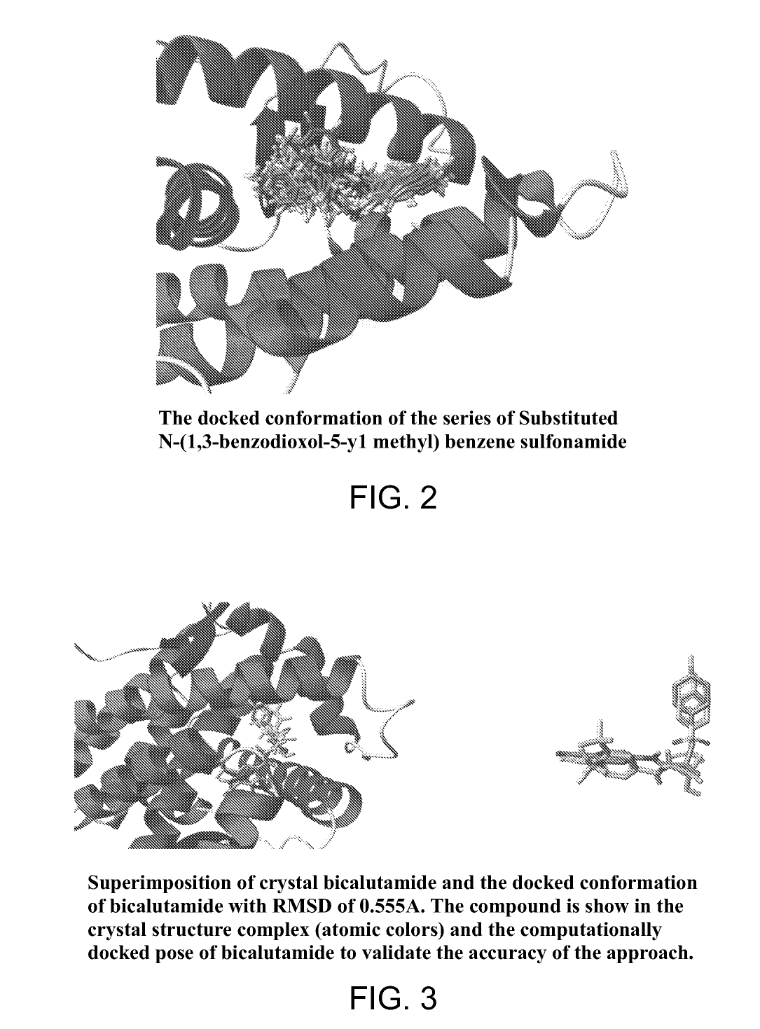

Using this same approach and the 3D pharmacophore triplet hypothesis (discussed above) another cluster of our in house library was screened for compounds similar to our active series resulting in the identification of another 40 compounds selected based on diversity of the R groups substitution pattern around our chemotype A lead scaffold. Through luciferase assays, these 40 compounds were screened at 50 μM for their ability to inhibit DHT stimulated AR transcriptional activity. Out of this structure-activity-relationship (SAR) series of 40 compounds 27 showed promising AR antagonist activity. Dose response studies were carried out for the 50 substituted N-(1,3-benzodioxol-5-ylmethyl)benzene sulfonamide derivatives. Their docked conformers of the compounds are represented in FIG. 2.

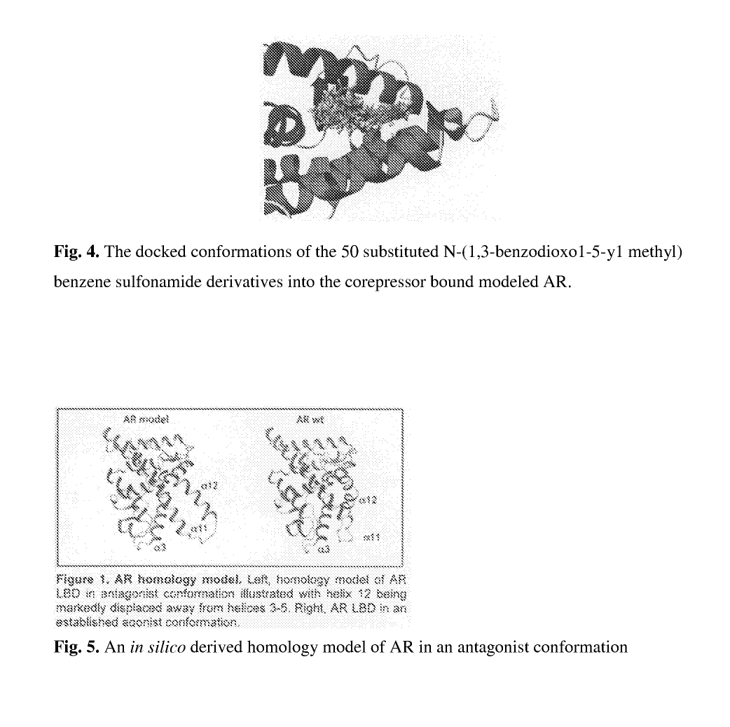

AR antagonist R-bicalutamide docked into the modeled AR protein and revealed interesting interactions in contrast to those interactions observed in the mutant W741L AR model (PDB ID code 1Z95) as illustrated in FIG. 3. The significance of these interactions within the ligand-protein interaction interface are nicely discussed by Bohl et al. In the docking studies with the antagonized conformation of the AR, observed was the hydrogen bonding interaction between cyano group in the A ring of the bicalutamide with the amino side chain hydrogen atom of the ARG752 and GLN711 residues. In addition to contribution for the hydrophobic interactions, trifluoromethy group forms hydrogen bond with the amino side chain hydrogen atom of VAL746 situated in the hydrophobic pocket. The amide side chain hydrogen atom of residue GLN783 forms hydrogen bond with the chiral hydroxyl group of the R-bicalutamide. The latter two interactions with VAL746 and GLN783 are not represented in the mutant AR-bicalutamide model and are crucial in binding the AR in the antagonistic form. Substituted N-(1,3-benzodioxol-5-yl methyl)benzene sulfonamide derivatives docked have a unique interaction in comparison to bicalutamide though they have a common interacting residue GLN783.

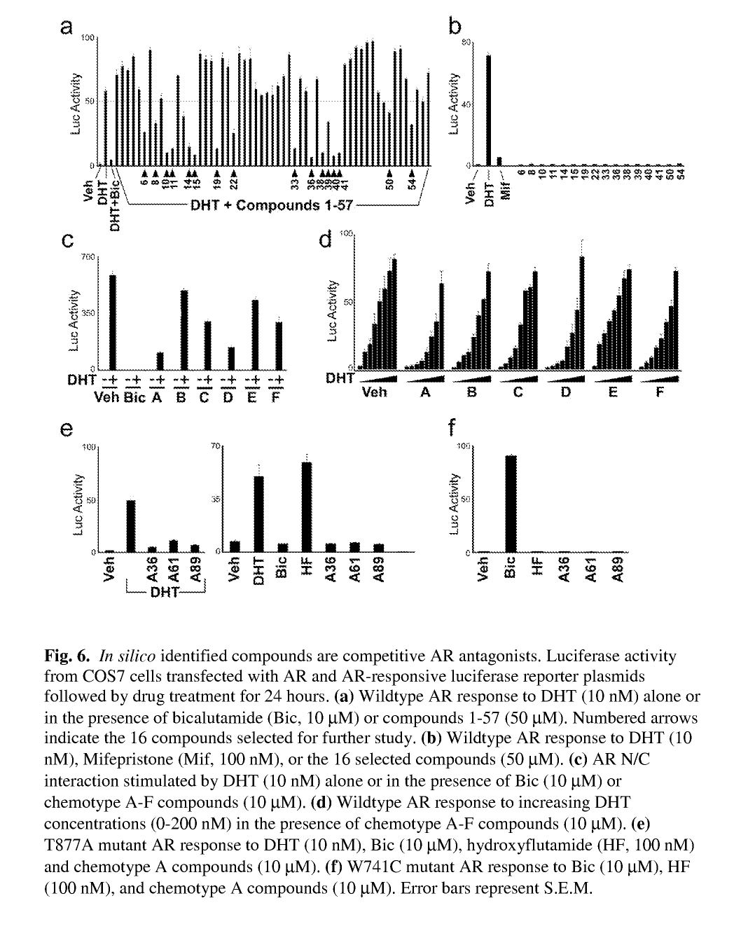

To further understand our functional data and to assemble hypothesizes regarding the possible mechanisms of action (MOA) for these compounds were using additional structure-based docking identifying the interaction pattern between the identified AR antagonists and the LBD of the AR. The set of identified compounds belonging to substituted N-(1,3-benzodioxol-5-yl methyl)benzene sulfonamide derivatives (Chemotype A) are docked into the LBD pocket of the AR (FIG. 4). The analysis of the docked pose of the most active compounds 36 (N-(1,3-benzodioxol-5-ylmethyl)-3-fluorobenzenesulfonamide), 61 (N-(1,3-benzodioxol-5-ylmethyl)-2,4-difluorobenzenesulfonamide) and 89 (N-(1,3-benzodioxol-5-ylmethyl)-4-ethoxy-2,5-difluorobenzenesulfonamide) reveal the unique interaction with the residues of the LBD. The compound 36 forms hydrogen bond with the residues ASN705 and GLN783. One of the oxygen in benzodioxazole ring forms hydrogen bond with side chain amine group of the ASN705 residue present in the N-terminal region of helix 3. Two sulfonyl oxygen atoms interact with side chain amine group of the GLN783 residue located at the N-terminal region of helix 7. Compound 61 forms hydrogen bonding interaction with the similar residues ASN705 and GLN783, through the oxygen atom in the benzodioxazole ring and with one of the sulfonyl oxygen atom. The compound 89 maintains all the interactions of the compound 61 and also interacts additionally with the carbonyl group of the GLN783 side chain through the amino hydrogen atom in the ligand. The presence of oxy ethy group (—OCH₂CH₃) at the para position of the benzenesulfonamide rings in compound 89 shift aryl ring position perpendicular to the benzodioxazole ring. The substituted 4-ethoxy-2,5-difluorobenzene aryl ring might be involved in hydrophobic interaction with the residues LEU707, MET745 and MET749 that forms the hydrophobic cleft of the ligand binding domain as illustrated in (FIG. 4).

Opportunity and Potential Impact

Multiple therapeutic targets have been identified in PCa and other cancers, but the pipeline of target-selective drugs that inhibit these proteins remains limited. The paucity of clinical compounds has been attributed to the recognized limitations of the tools currently employed, limited chemical diversity within the compound repositories that are screened, as well as the limited target space that has been deemed “druggable”. The currently described in silico CADDD platform mitigates several of these limitations, allowing one to screen 10⁶-10⁷ compounds in target classes that are within what has been defined as the “expanded druggable genome” with a focus on protein-protein or protein-DNA interfaces. This novel CADDD platform has identified novel antagonists for two compelling therapeutic targets in PCa, androgen receptor (AR) and ERG.

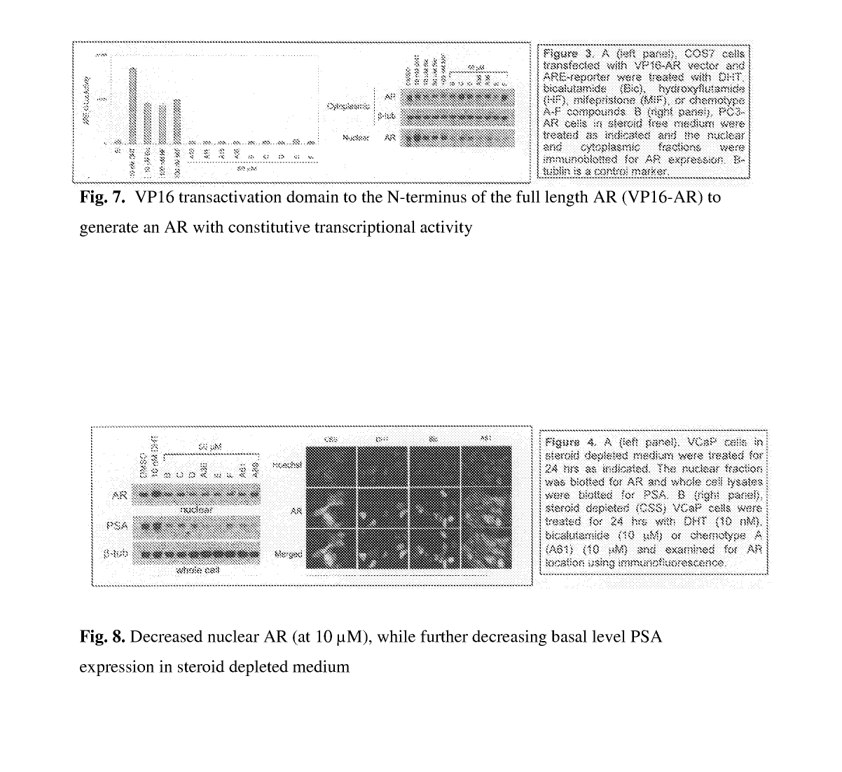

While the AR is a well validated therapeutic target, there remains a paucity of structural information detailing its conformation in an antagonized state. To date X-ray crystallography studies have only been successful in generating structures of wild-type AR bound to agonists or mutant ARs bound to antagonists that function as agonists as a result of these mutations. Such limitations have been overcome by this invention through the use of an innovative CADDD platform that has been developed. Computational methods like these are playing an increasingly significant role in academic as well as industry drug discovery initiatives. A homology modeling strategy powered by a wealth of structure/function data from the AR and other nuclear hormone receptor family members provided an in silico derived homology model of AR in an antagonist conformation (FIG. 5). Briefly, the AR homology model was developed using a five stage iterative construction process: 1) identification of a suitable template(s) or reference sequence(s) of known structure (template selection); 2) optimal alignment of the target sequence(s) to the template sequence (target-template alignment); 3) model construction of structurally conserved regions using the template(s) structures; 4) modeling of side chains and regions of sequence divergence; and 5) refinement of the model structure through conformational sampling and evaluation. Importantly it is well established that when sequences with greater than 40% identity to the template sequence (AR) are modeled (as was done here), 90% of the mainchain atoms are within an RMS error of ˜1 A (Fiser. 2004). Using structure/function details provided by other nuclear receptor LBD complexes combined with the structural insight provided by the model in FIG. 5, various interfacial “hot spots” have been defined, which allow for the identification and rational optimization of small molecule antagonists using structure-based virtual screens (SBVSs) of small molecules. These target-dependent in silico screens offer a unique, accelerated strategy for the identification of target-selective “hits”. When the SBVSs were partnered with ligand-dependent pharmacophore screens using the pharmacophore models that represent requisite, non-steroidal steric and electronic features present in known active antagonists (including those noted), the above-described CADDD platform identified 57 non-steroidal compounds that recognized and docked into the AR LBD in this antagonized conformation.

Functional Validation of in Silico Identified Antagonists

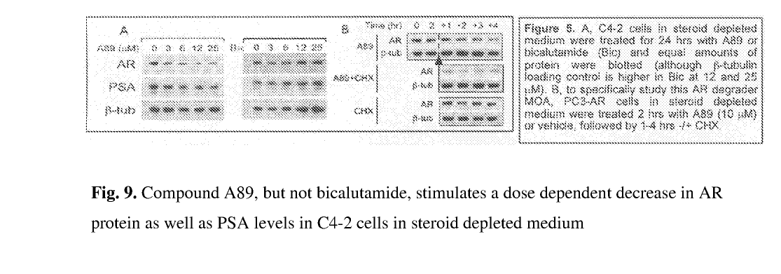

The 57 compounds were initially evaluated for their ability to inhibit DHT-stimulated AR activity in COS7 cells transfected with an AR expression vector, AR-responsive firefly luciferase reporter plasmid, and a constitutive Renilla luciferase control plasmid. At an initial concentration of 50 μM, 16 compounds demonstrated >50% inhibition of DHT stimulated AR reporter activity, with no significant effect on the internal Renilla luciferase control (FIG. 6a). Several of these compounds inhibited AR activity 80-90%, which was comparable to the effects of 10 μM bicalutamide, while none of 16 compounds had demonstrable agonist activity (FIG. 6b). The 16 compounds provided an initial structure activity relationship (SAR) series that was used to further refine our ligand-based pharmacophores for subsequent ligand-based virtual screens (LBVSs). These additional CADDD screens identified 40 additional chemotype A analogs, several of which (including A61 and A89) had increased potency in antagonizing AR activity in these luciferase reporter assays (data not shown).

To more directly confirm that the compounds did not induce an agonist conformation, we studied the ability of the compounds to stimulate the interaction between the AR NTD and the LBD. This AR N/C interaction is mediated by the LxxLL-like motif in the AR NTD binding to the agonist-induced coactivator binding site in the LBD. Cells were co-transfected with expression vectors encoding the VP16 transactivation domain fused to the AR NTD (VP16-AR NTD), the GAL4 DBD fused to the AR LBD (GAL4-AR LBD), and a GAL4 regulated luciferase reporter. As expected, the AR N/C interaction was stimulated by DHT, but not by the antagonist bicalutamide (FIG. 6c). Significantly, none of the compounds stimulated the N/C interaction. Moreover, they inhibited DHT-stimulated AR N/C interaction to varying degrees consistent with their potency as AR antagonists.

To confirm that the compounds were binding within the steroid binding pocket of the LBD, we evaluated the competitive nature of this inhibition in the presence of increasing DHT concentrations. Cells transfected with AR and luciferase reporter plasmids were treated with increasing concentrations of DHT (0-200 nM) in the presence of 10 μM of compounds. Each of the compounds at this concentration inhibited AR activity at the lowest level of DHT (FIG. 6d). Significantly, this inhibition could be abrogated by increasing the DHT concentration, consistent with competitive binding to the LBD steroid binding pocket. To further validate the predicted positioning of the compounds in the steroid binding pocket, we also examined the effects of established mutations within the LBD. The well-characterized T877A mutation increases the size of the steroid binding pocket, which mitigates the ability of hydroxyflutamide to distort this variant LBD allowing this antagonist to function as a strong agonist. Our benchmarked modeling studies predict that the compounds do not reposition T877 (data not shown). Consistent with this prediction, these compounds retain their antagonist activity for the T877A mutant AR and importantly do not gain the agonist activity demonstrated by hydroxyflutamide (FIG. 6e). The compounds also failed to activate the W741C mutant AR, which can be strongly activated by bicalutamide. These findings are consistent with our modeling data that predict chemotype A antagonist activities are not dependent on displacement of W741 (FIG. 6f).

Antagonists Prevent Chromatin Binding and Decrease AR Nuclear Localization.

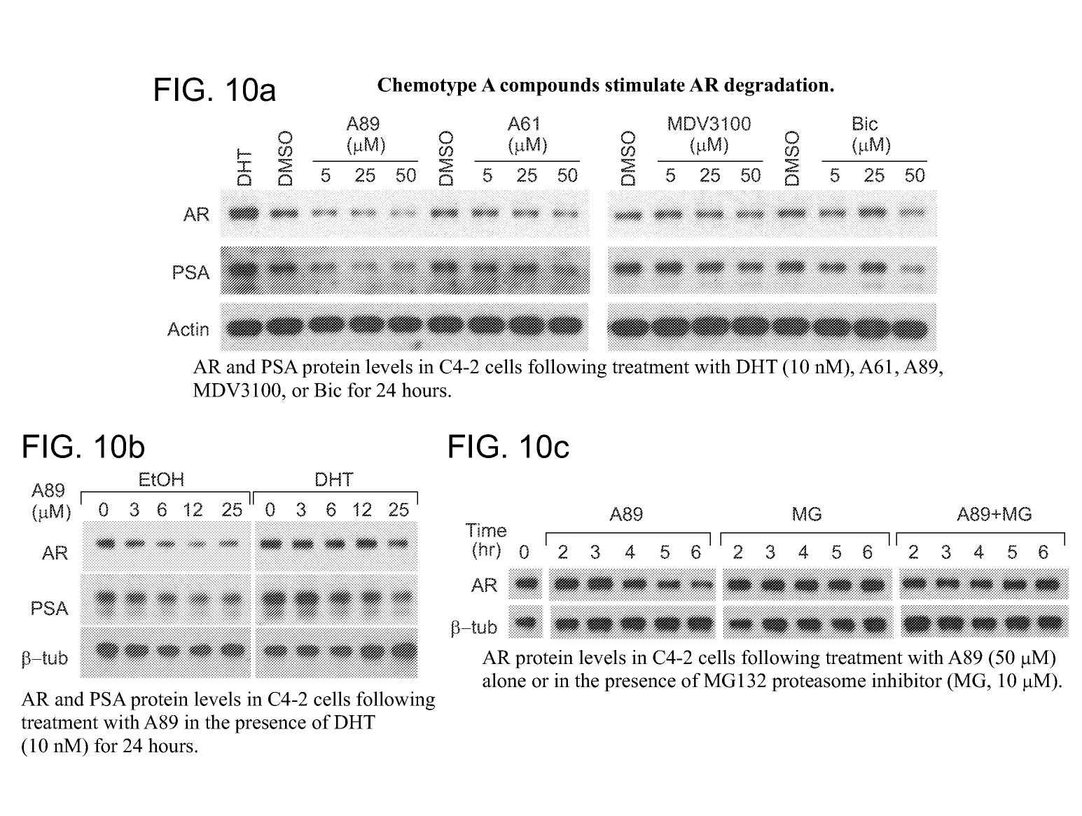

Currently available AR antagonists including bicalutamide impair coactivator binding and may enhance corepressor recruitment, but these compounds do not prevent AR nuclear localization/chromatin binding and thus may acquire some partial agonist activity in CRPC cells (Masiello et al., 2002; Tran et al., 2009). To address whether the compounds stimulate AR binding to chromatin, we fused the VP16 transactivation domain to the N-terminus of the full length AR (VP16-AR) to generate an AR with constitutive transcriptional activity. Consistent with previous data (Masiello et al., 2002), this VP16-AR yielded strong reporter activity in response to DHT, bicalutamide, hydroxyflutamide, and mifepristone (FIG. 7A). However, in marked contrast, there was no transcriptional activity induced by any of our compounds. Using nuclear/cytoplasmic cell extracts from PC3 cells stably transfected with AR, we further demonstrated that the compounds prevented nuclear AR accumulation, and decreased the ratio of nuclear/cytoplasmic AR (FIG. 7B).

Compounds Suppress AR Nuclear Localization and Enhance AR Degradation in PCa Cells.

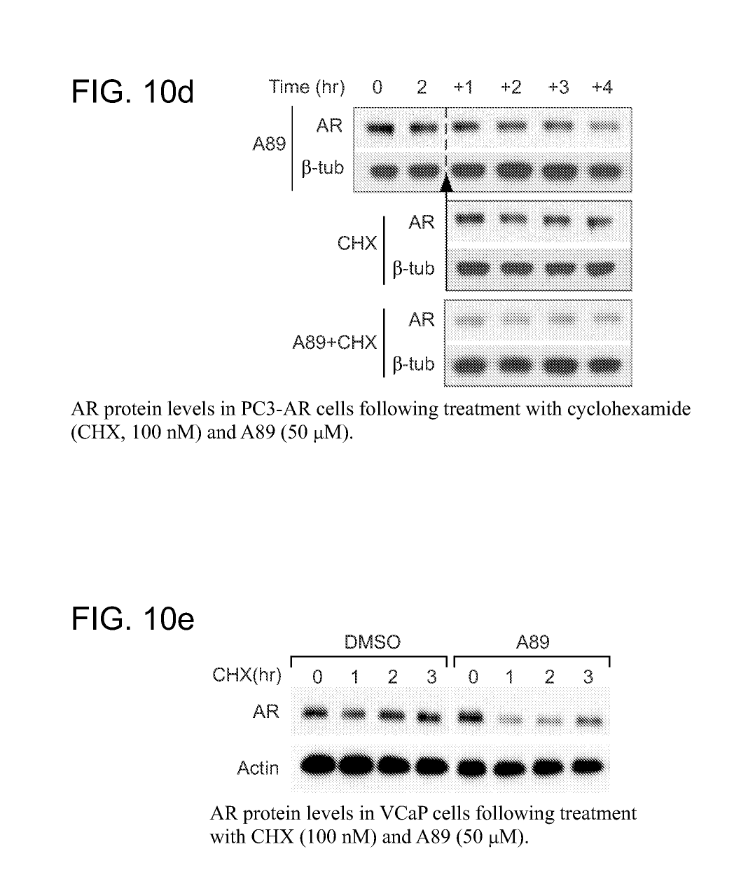

VCaP cells, derived from a CRPC vertebral metastasis, express substantial levels of AR and AR regulated genes such as PSA even when cultured in steroid depleted medium. Significantly, in addition to blocking DHT stimulated PSA expression (not shown), each of the compounds (including 2^{nd} generation chemotype A compounds, A61 and A89) significantly decreased nuclear AR (at 10 μM), while further decreasing basal level PSA expression in steroid depleted medium (FIG. 8A). In additional studies focused on chemotype A compounds, we used immunofluorescence to assess AR nuclear localization. While VCaP cells exhibit some nuclear AR staining when grown in steroid depleted media (CSS medium), the nuclear AR levels were markedly increased by DHT and by bicalutamide (Bic) (FIG. 8B). In contrast, and consistent with the biochemical fractionation studies noted above, basal nuclear AR was decreased by A61.

Chemotype A Compounds Enhance AR Protein Degradation.

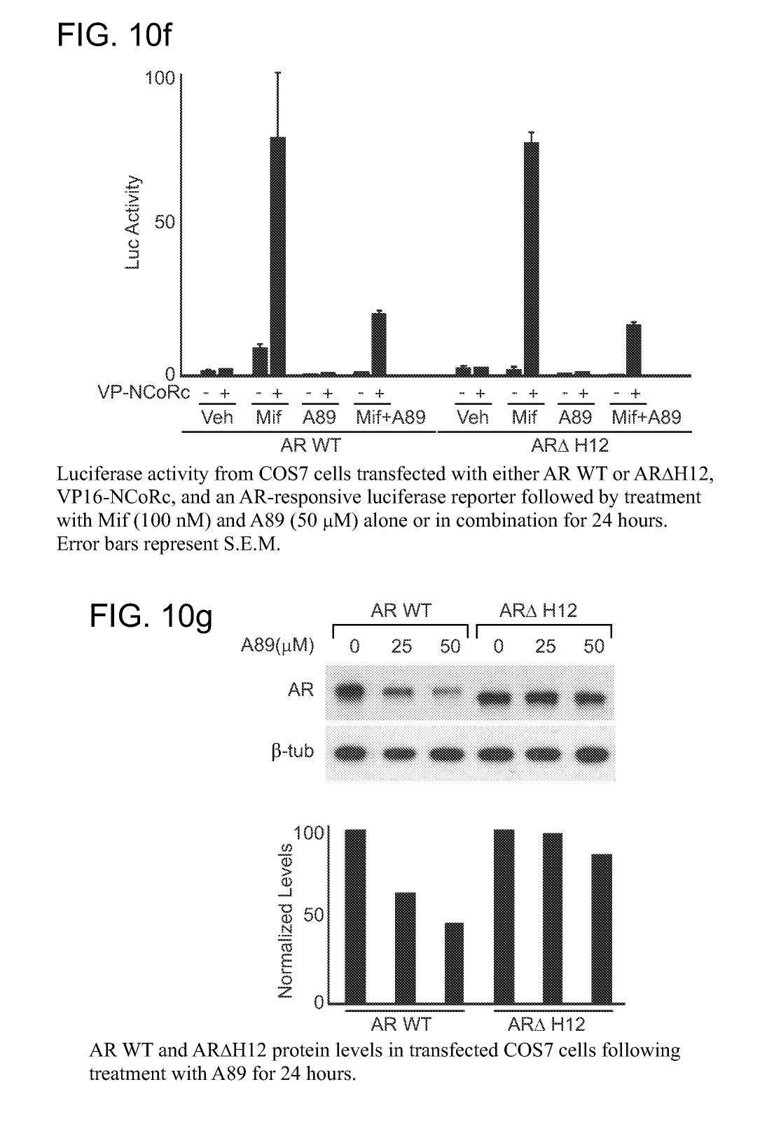

Total cellular AR protein levels were also decreased to varying degrees in response to each of the chemotype A compounds (10 μM) in VCaP cells and C4-2 cells (derived from a LNCaP PCa xenograft that relapsed after castration). It should be noted that while both LNCaP and C4-2 cells express a mutant AR (T877A), our compounds inhibit this mutant AR in reporter gene assays (data not shown). Subsequent mechanistic studies have focused on the 2^{nd} generation chemotype A compounds, A61 and A89, based on their relatively higher potency for the AR in reporter assays (not shown). FIG. 9A shows that compound A89, but not bicalutamide, stimulates a dose dependent decrease in AR protein as well as PSA levels in C4-2 cells in steroid depleted medium. Significantly, the decrease in AR was blocked in the presence of a proteasome inhibitor, indicating increased degradation (not shown). To more definitively assess the AR protein degradation as an MOA for wild-type AR (independent of effects on endogenous AR mRNA), an AR negative PCa cell line (PC3 cells) that was stably transfected with wild-type AR under the control of a heterologous CMV promoter (PC3-AR cells) was examined. As in C4-2 and VCaP cells, compound A89 (10 μM) decreased AR protein within 4 hours (FIG. 9B). Moreover, when cycloheximide (CHX) was added to block new protein synthesis, there was a rapid (within ˜1 hour) and dramatic decrease in total AR protein in the A89 treated cells, supporting the conclusion that A89 targets the AR for rapid degradation (it is not yet clear whether the low level of AR protein remaining after 4 hours of CHX+A89 treatment is a distinct, more stable pool of AR that is not bound by A89). In further MOA studies it was found that chemotype A binding to the AR LBD is helix 12 independent [consistent with our model of AR in the antagonistic conformation (FIG. 5)], while the signal for degradation is helix 12 dependent, which suggests that a surface on the displaced helix 12 is involved in this degradation MOA (not shown).

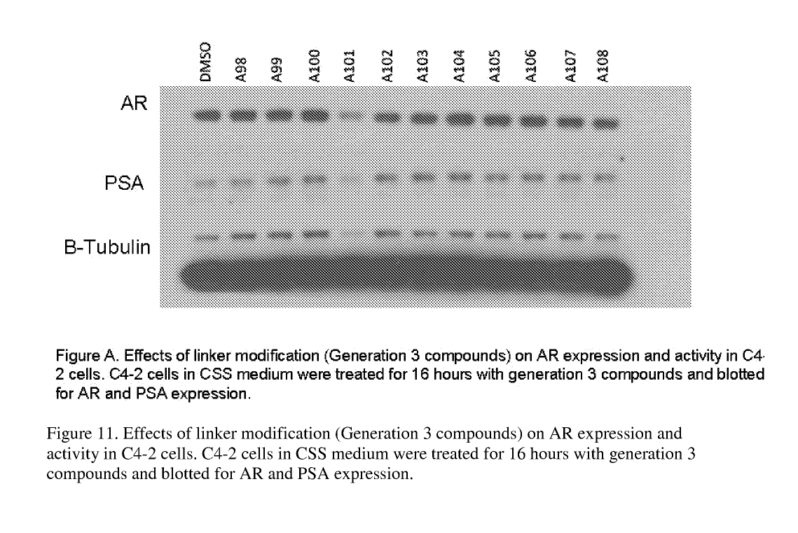

Similar to the results in VCaP cells, our chemotype A lead compounds A61 and A89 reduced nuclear AR levels and PSA expression in another CRPC cell line (C4-2 cells, derived from a castration resistant LNCaP xenograft) cultured in steroid depleted medium (data not shown). Significantly, in the C4-2 cells both A61 and A89 caused a noteworthy decrease in total AR protein levels that correlated with a decrease in basal PSA (FIG. 10a). MDV3100 and bicalutamide showed no clear effect on basal PSA or AR protein levels at concentrations (5 μM) that suppress DHT stimulated AR activity, while modest decreases were observed at higher drug concentrations (25-50 μM). The decline in AR protein in response to chemotype A appeared to be a direct consequence of AR antagonist binding as it was competitively blocked in the presence of DHT (FIG. 10b). This decrease in AR protein was also prevented by treating the cells with proteasome inhibitors (MG115 and MG132), implicating AR protein degradation as an advantageous chemotype A mechanism of action (FIG. 10c).

As LNCaP and C4-2 cells express the T877A mutant AR, we next examined PC3-AR cells expressing wildtype AR. As observed in C4-2 cells, A89 caused a decrease in AR protein levels (FIG. 10d). To further validate that this was due to increased AR protein degradation, we pretreated these cells with A89 for 2 hours and then blocked new protein synthesis with cycloheximide (CHX). Significantly, AR protein levels declined markedly in A89 treated cells following 1 hour treatment with CHX as compared to the effects of CHX alone in vehicle treated control cells (FIG. 10d). We finally examined VCaP cells. Interestingly, while androgen increases AR stability in these cells, AR protein levels decrease due to a due to a marked decline in AR mRNA (Cai et al., 2009; Yu et al., 2010). Nonetheless, using CHX we confirmed that A89 increased degradation of endogenous wildtype AR in VCaP cells as similarly seen in C4-2 and PC3-AR cells (FIG. 10e).

### Chemotype A-Induced AR Degradation is Helix 12 Dependent

The ERα antagonist fulvestrant (Faslodex) represents a novel class of estrogen receptor downregulators that appear to mediate ERα degradation by specifically repositioning helix 12. To assess the role of helix 12 in chemotype A-mediated AR degradation, we first determined if helix 12 is required for A89 binding. As this helix is required for AR agonist activity, we could not directly measure competitive antagonist activity on a truncated AR lacking helix 12. However, we have shown previously that the AR antagonist mifepristone binds to the AR and recruits the corepressor protein NCoR in the absence of helix 12. Given these data we used this mifepristone-stimulated NCoR recruitment as a readout to assay competitive A89 binding to the LBD. COS7 cells were co-transfected with expression vectors for either intact wildtype AR (AR WT) or an M886X variant AR in which the AR was truncated between helices 11 and 12 (AR ΔH12) along with an expression vector encoding the NCoR C-terminal AR binding region fused to the VP16 transactivation domain (VP16-NCoRc) and an AR-responsive luciferase reporter.

Consistent with our previous results, mifepristone strongly enhanced the interaction of VP16-NCoRc with both AR WT and AR ΔH12 (FIG. 100. Significantly, A89 did not stimulate VP16-NCoRc recruitment, and it suppressed mifepristone-mediated VP16-NCoRc recruitment equivalently on both AR WT and AR ΔH12, indicating that A89 binding is not dependent on helix 12. We next transiently transfected COS7 cells with AR WT or AR ΔH12 plasmids and assayed for AR expression levels in response to A89. As expected, AR WT protein levels decreased in a dose dependent manner following treatment with A89 FIG. 10g). In contrast, AR ΔH12 protein levels were not clearly decreased by A89 treatment. Taken together, these data strongly suggest that the A89-induced repositioning of helix 12 generates a signal that targets AR for degradation.

Chemotype A Compounds are AR Antagonists In Vivo and are Efficacious in CRPC Xenografts.

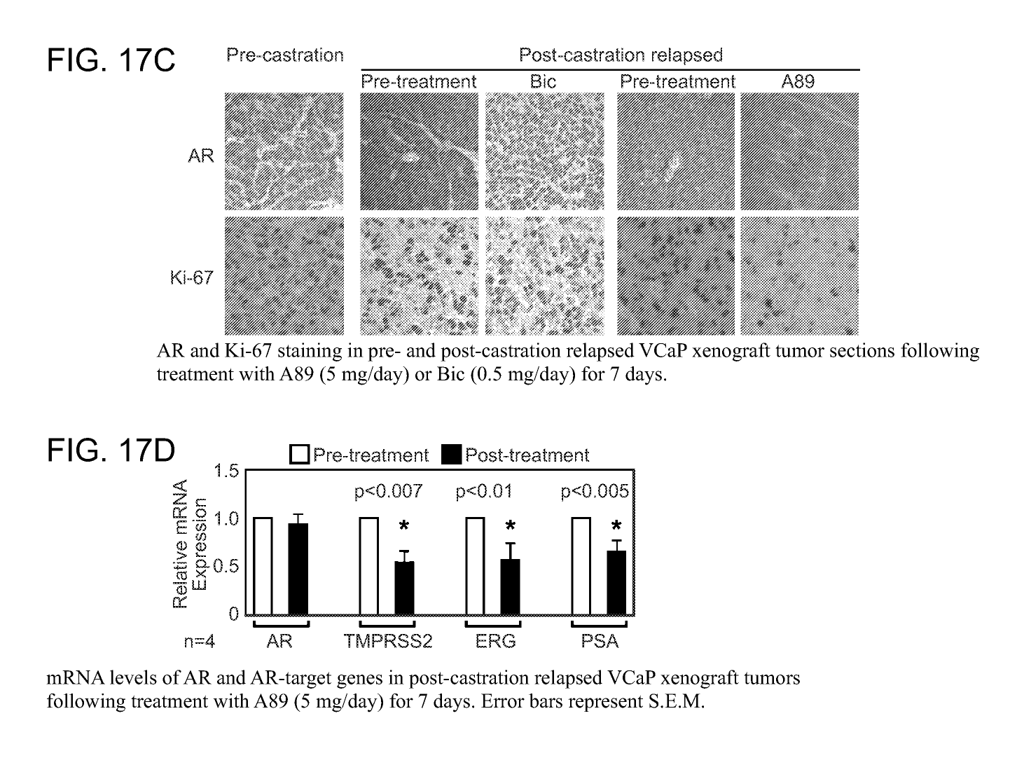

To evaluate the activity of the chemotype A compounds in vivo we first treated adult male mice with compound A89 in a dose ranging experiment from 0.5 mg/day to 10 mg/day via intraperitoneal injection for 7 days. While all mice remained outwardly healthy, the seminal vesicles underwent dose-dependent involution (a biomarker of AR antagonist activity) beginning at 2.5 mg/day (FIG. 17a). Compound A89 similarly caused atrophy of the androgen sensitive prostate epithelium (FIG. 17b). We next evaluated A89 in VCaP xenograft tumors that had relapsed following an initial response to castration, which is a model of CRPC. Consistent with our previous data, these relapsed VCaP xenografts expressed substantial nuclear and cytoplasmic AR and were refractory to bicalutamide, which actually enhanced nuclear AR expression (FIG. 17c). In contrast, A89 substantially decreased nuclear AR staining and decreased tumor cell proliferation as evidenced by Ki-67 staining (FIG. 17c). Analysis of mRNA extracted from biopsies of relapsed VCaP tumors pre- and post treatment with A89 further demonstrated a significant decrease in expression of androgen regulated genes including TMPRSS2, ERG (from the TMPRSS2:ERG fusion gene in VCaP), and PSA (FIG. 17d). Similar results were obtained using a second chemotype A lead, A61 (data not shown).

In summary, we have developed a broadly applicable CADDD platform for small molecule drug discovery that has been benchmarked and validated through the identification of a series of AR antagonists that are both chemically novel and mechanistically unique. Significantly, despite the paucity of structural data for the AR LBD in an antagonized conformation, we were able to generate a refined homology model predictive of this antagonistic conformation for use in our target driven SBVSs. LBVSs that used stringent ligand-dependent pharmacophore models developed around a training set of AR antagonists, which were benchmarked for pure antagonist descriptors against a series of AR agonists, facilitated the rapid identification of a proposed small molecule hit list. We then secondarily screened this hit list using our target-dependent SBVSs, and finally filtered the proposed hit list using in silico ADMET models. This platform approach facilitated the enrichment of small molecules optimized for AR antagonism and drug-like properties. Remarkably, preliminary functional studies demonstrated that the 16 most active compounds were all competitive antagonists devoid of agonist activity and the ability to stimulate AR N/C terminal interaction that is associated with AR activation. Moreover, the compounds all decreased nuclear AR levels and prevented chromatin binding as assessed using a constitutively active VP16-AR fusion protein that is robustly recruited by currently available AR antagonists. These data support that this virtual screening platform was able to successfully identify several small molecules representative of distinct chemical architectures that all stabilize a similar or identical antagonist conformation.

Studies aimed at defining the mechanism of action for these compounds revealed that compounds of the invention stabilize a conformation of the AR that is targeted for proteasome mediated degradation. Newly synthesized AR protein undergoes multiple rounds of Hsp90 dependent refolding to maintain a conformation competent to bind androgen, but in the absence of ligand the AR appears to undergo polyubiquitination and proteasome-mediated degradation. Structural features responsible for targeting the AR for protein degradation remain unclear, but our data support that the predicted chemotype A-mediated repositioning of helix 12 may expose a surface on this helix that is recognized by the degradation machinery, as appears to be the case for the fulvestrant-liganded ERα. Based on these findings, we propose that the functional consequences of antagonist binding may be dictated by the precise localization of helix 12, and that antagonist mediated stabilization of this helix in the degradation conformer may drive AR protein degradation. Importantly, this chemotype A-dependent degradation event may represent a mechanism of action advantage allowing these antagonists to compete with high affinity intratumorally synthesized AR ligands that contribute to CRPC.

Overall, these studies have resulted in the identification of a novel series of AR antagonists that uniquely incorporate the following mechanisms of action against AR reactivated in CRPC: 1) blocking of androgen binding to AR, 2) prevention of AR nuclear translocation, 3) inhibition of AR DNA binding, and 4) stimulation of AR protein degradation. Moreover, these findings exemplify the power and utility of novel computational screening platforms that, when applied in synergy with strong biological insights into the signaling mechanisms of target molecules, serve as essential tools for effectively translating such insights into real-world pharmaceuticals.

### DEFINITIONS

Listed below are definitions of various terms used to describe this invention. These definitions apply to the terms as they are used throughout this specification and claims, unless otherwise limited in specific instances, either individually or as part of a larger group. The number of carbon atoms in a hydrocarbyl substituent can be indicated by the prefix “Cₓ-C_{y},” where x is the minimum and y is the maximum number of carbon atoms in the substituent. Likewise, a Cₓ chain means a hydrocarbyl chain containing x carbon atoms.

If a linking element in a depicted structure is “absent”, then the left element in the depicted structure is directly linked to the right element in the depicted structure. For example, if a chemical structure is depicted as X-(L)ₙ-Y wherein L is absent or n is 0, then the chemical structure is X—Y.

The term “alkyl” as used herein, refers to a saturated, straight- or branched-chain hydrocarbon radical. For example, “C₁-C₈ alkyl” contains from one to eight carbon atoms. Examples of alkyl radicals include, but are not limited to, methyl, ethyl, propyl, isopropyl, n-butyl, tert-butyl, neopentyl, n-hexyl, heptyl, octyl radicals and the like.

The term “alkenyl” as used herein, denotes a straight- or branched-chain hydrocarbon radical containing one or more double bonds. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds. For example, “C₂-C₈ alkynyl” contains from from two to eight carbon atoms. Representative alkynyl groups include, but are not limited to, for example, ethynyl, 1-propynyl, 1-butynyl, heptynyl, octynyl and the like.

The term “alkoxy” refers to an —O-alkyl moiety.

The term “aralkyl,” or “arylalkyl,” as used herein, refers to an alkyl residue attached to an aryl ring. Examples include, but are not limited to, benzyl, phenethyl and the like.

The term “cycloalkyl” denotes a monovalent group derived from a monocyclic or polycyclic saturated carbocyclic ring compound. Examples of cycloalkyl include, but not limited to, cyclopropyl, cyclobutyl, cyclopentyl, cyclohexyl, bicyclo[2.2.1]heptyl, and bicyclo[2.2.2]octyl and the like.

The terms “carbocycle” or “carbocyclic” or “carbocyclyl” refer to a saturated (e.g., “cycloalkyl”), partially saturated (e.g., “cycloalkenyl” or “cycloalkynyl”) or completely unsaturated (e.g., “aryl”) ring system containing zero heteroatom ring atom. A carbocyclyl may be, without limitation, a single ring, or two or more fused rings, or bridged or spiro rings. A carbocyclyl may contain, for example, from 3 to 10 ring members (i.e., C₃-C₁₀carbocyclyl, such as C₃-C₁₀cycloalkyl). A substituted carbocyclyl may have either cis or trans geometry. Representative examples of carbocyclyl groups include, but are not limited to, cyclopropyl, cyclobutyl, cyclopentyl, cyclohexyl, cycloheptyl, cyclooctyl, cyclopentenyl, cyclopentadienyl, cyclohexadienyl, adamantyl, decahydro-naphthalenyl, octahydro-indenyl, cyclohexenyl, phenyl, naphthyl, fluorenyl, indanyl, 1,2,3,4-tetrahydro-naphthyl, indenyl, isoindenyl, bicyclodecanyl, anthracenyl, phenanthrene, benzonaphthenyl (also known as “phenalenyl”), decalinyl, and norpinanyl and the like. A carbocyclyl group can be attached to the parent molecular moiety through any substitutable carbon atom of the group.

The term “aryl” refers to an aromatic carbocyclyl containing from 6 to 14 carbon ring atoms. Non-limiting examples of aryls include phenyl, naphthalenyl, anthracenyl, and indenyl and the like. An aryl group can be connected to the parent molecular moiety through any substitutable carbon atom of the group.

The term “heteroaryl” means an aromatic heterocyclyl typically containing from 5 to 18 ring atoms. A heteroaryl may be a single ring, or two or more fused rings. Non-limiting examples of five-membered heteroaryls include imidazolyl; furanyl; thiophenyl (or thienyl or thiofuranyl); pyrazolyl; oxazolyl; isoxazolyl; thiazolyl; 1,2,3-, 1,2,4-, 1,2,5-, and 1,3,4-oxadiazolyl; and isothiazolyl. Non-limiting examples of six-membered heteroaryls include pyridinyl; pyrazinyl; pyrimidinyl; pyridazinyl; and 1,3,5-, 1,2,4-, and 1,2,3-triazinyl. Non-limiting examples of 6/5-membered fused ring heteroaryls include benzothiofuranyl, isobenzothiofuranyl, benzisoxazolyl, benzoxazolyl, purinyl, and anthranilyl. Non-limiting examples of 6/6-membered fused ring heteroaryls include quinolinyl; isoquinolinyl; and benzoxazinyl (including cinnolinyl and quinazolinyl).

The term “heterocycloalkyl” refers to a non-aromatic 3-, 4-, 5-, 6- or 7-membered ring or a bi- or tri-cyclic group fused system, where at least one of the ring atoms is a heteroatom, and where (i) each 5-membered ring has 0 to 1 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl and the like.

The terms “heterocyclic” or “heterocycle” or “heterocyclyl” refer to a saturated (e.g., “heterocycloalkyl”), partially unsaturated (e.g., “heterocycloalkenyl” or “heterocycloalkynyl”) or completely unsaturated (e.g., “heteroaryl”) ring system, where at least one of the ring atoms is a heteroatom (i.e., nitrogen, oxygen or sulfur), with the remaining ring atoms being independently selected from the group consisting of carbon, nitrogen, oxygen and sulfur. A heterocyclyl group can be linked to the parent molecular moiety via any substitutable carbon or nitrogen atom in the group, provided that a stable molecule results. A heterocyclyl may be, without limitation, a single ring. Non-limiting examples of single-ring heterocyclyls include furanyl, dihydrofuranyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazoly, pyranyl, dihydropyranyl, pyridinyl, piperidinyl, pyridazinyl, pyrimidinyl, pyrazinyl, piperazinyl, triazinyl, isoxazinyl, oxazolidinyl, isoxazolidinyl, oxathiazinyl, oxadiazinyl, morpholinyl, azepinyl, oxepinyl, thiepinyl, or diazepinyl. A heterocyclyl may also include, without limitation, two or more rings fused together, such as, for example, naphthyridinyl, thiazolpyrimidinyl, thienopyrimidinyl, pyrimidopyrimidinyl, or pyridopyrimidinyl. A heterocyclyl may comprise one or more sulfur atoms as ring members; and in some cases, the sulfur atom(s) is oxidized to SO or SO₂. The nitrogen heteroatom(s) in a heterocyclyl may or may not be quaternized, and may or may not be oxidized to N-oxide. In addition, the nitrogen heteroatom(s) may or may not be N-protected.

The term “alkylamino” refers to a group having the structure —NH(C₁-C₁₂ alkyl) where C₁-C₁₂ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

The terms “hal,” “halo” and “halogen,” as used herein, refer to an atom selected from fluorine, chlorine, bromine and iodine.

The term “oxo” as used herein, refers to an oxygen that is attached to a carbon, preferably by a double bond (e.g., carbonyl).

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The term “optionally substituted”, refers to groups that are substituted or unsubstituted by independent replacement of one, two, or three or more of the hydrogen atoms thereon with substituents including, but not limited to:

alkyl, alkenyl, alkynyl, cycloalkyl, aryl, heterocycloalkyl, heteroaryl, arylalkyl, heteroarylalkyl,

—F, —Cl, —Br, —I,

—OH, protected hydroxy, oxygen, oxo,

—NO₂, —CN,

—NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino,

—O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl,

—C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl,

—CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl,

—OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH— aryl, —OCONH— heteroaryl, —OCONH— heterocycloalkyl,

—NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂— aryl, —NHCO₂— heteroaryl, —NHCO₂— heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl,

—C(NH)NH—C₁-C₁₂-alkyl, —C(NH)NH—C₂-C₁₂-alkenyl, —C(NH)NH—C₂-C₁₂-alkenyl, —C(NH)NH—C₃-C₁₂-cycloalkyl, —C(NH)NH-aryl, —C(NH)NH-heteroaryl, —C(NH)NH-heterocycloalkyl,

—S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl-SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH— heteroaryl, —SO₂NH— heterocycloalkyl,

—NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl,

—CH₂NH₂, —CH₂SO₂CH₃, -alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, -methoxymethoxy, -methoxyethoxy, —SH, —S— alkyl, —S— alkenyl, —S— alkynyl, —S-cycloalkyl, —S-aryl, —S-heteroaryl, —S-heterocycloalkyl, or methylthiomethyl.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

Treat”, “treating” and “treatment” refer to a method of alleviating or abating a disease and/or its attendant symptoms.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating disorders through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Pharmaceutical Compositions

In another aspect, the invention provides a pharmaceutical composition comprising a compound of formula I, or any other compound as described herein, or a pharmaceutically acceptable ester, salt, or prodrug thereof, together with a pharmaceutically acceptable carrier.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other anti-proliferative, anti-cancer, immunomodulatory or anti-inflammatory substances. Where the compounds of the invention are administered in conjunction with other therapies, dosages of the co-administered compounds will of course vary depending on the type of co-drug employed, on the specific drug employed, on the condition being treated and so forth.

Combination therapy includes the administration of the subject compounds in further combination with other biologically active ingredients (such as, but not limited to, a second and different anticancer agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved chemotherapeutic agent, or it may be any one of a number of agents undergoing approval in the Food and Drug Administration that ultimately obtain approval for the treatment of protozoal infections and/or any disorder associated with cellular hyperproliferation. In certain other embodiments, the additional therapeutic agent is an anticancer agent, as discussed in more detail herein. In certain other embodiments, the compositions of the invention are useful for the treatment of protozoal infections. In the treatment of cancer or protein degradation disorders, the inventive compound may be combined with a proteasome inhibitor (e.g., bortezomib, R1 15777 FTI, MG132, NPI-0052, etc.). In the treatment of cancer or protein degradation disorders, the inventive compound may be combined with protein degradation inhibitor (e.g. another inventive compound, a tubacin-like compound, bortezomib, R1 15777 FTI, MG132, NPI-0052, SAHA, ¹⁶⁶Ho-DOTMP, arsenic trioxide, 17-AAG, MG 132, etc.).

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

The present invention encompasses pharmaceutically acceptable topical formulations of inventive compounds. The term “pharmaceutically acceptable topical formulation,” as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents {e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et ah, Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, IU. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide. fatty acid esters (e.g., isopropyl myristate, methyl laurate, glycerol monooleate, and propylene glycol monooleate) and N-methylpyrrolidine.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects (e.g., control of any adverse effects).

For example, other therapies or anticancer agents that may be used in combination with the inventive compounds of the present invention include, but not limited to, surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, antibodies, aptamers, siRNAs, oligonucletoides, enzyme, ion channel and receptor inhibitors or activators to name a-few), hyperthermia and cryotherapy, agents to attenuate any adverse effects (e.g., antiemetics), and other approved chemotherapeutic drugs, including, but not limited to, alkylating drugs (e.g., mechlorethamine, chlorambucil, Cyclophosphamide, Melphalan, Ifosfamide), antimetabolites (e.g., Methotrexate), purine antagonists and pyrimidine antagonists (e.g., 6-Mercaptopurine, 5-Fluorouracil, Cytarabile, Gemcitabine), spindle poisons (e.g., Vinblastine, Vincristine, Vinorelbine, Paclitaxel), podophyllotoxins (e.g., Etoposide, Irinotecan, Topotecan), antibiotics (Doxorubicin, Bleomycin, Mitomycin), nitrosoureas (e.g., Carmustine, Lomustine), inorganic ions (e.g., Cisplatin, Carboplatin), enzymes (e.g., Asparaginase), and hormones (e.g., Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/dmglistfrarne).

In certain embodiments, the pharmaceutical compositions of the present invention further comprise one or more additional therapeutically active ingredients (e.g., chemotherapeutic and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications, anti-pyretics, and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

The pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, disorders are treated or prevented in a subject, such as a human or other animal, by administering to the subject a therapeutically effective amount of a compound of the invention, in such amounts and for such time as is necessary to achieve the desired result. The term “therapeutically effective amount” of a compound of the invention, as used herein, means a sufficient amount of the compound so as to decrease the symptoms of a disorder in a subject. As is well understood in the medical arts a therapeutically effective amount of a compound of this invention will be at a reasonable benefit/risk ratio applicable to any medical treatment.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight (0.05 to 4.5 mg/m²). An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

In certain embodiments, a therapeutic amount or dose of the compounds of the present invention may range from about 0.1 mg/kg to about 500 mg/kg (about 0.18 mg/m² to about 900 mg/m²), alternatively from about 1 to about 50 mg/kg (about 1.8 to about 90 mg/m²). In g general, treatment regimens according to the present invention comprise administration to a patient in need of such treatment from about 10 mg to about 1000 mg of the compound(s) of this invention per day in single or multiple doses. Therapeutic amounts or doses will also vary depending on route of administration, as well as the possibility of co-usage with other agents.

Generally, the daily dosage of a compound of structural formula I may be varied over a wide range from 0.01 to 1000 mg per adult human per day. Most preferably, dosages range from 0.1 to 200 mg/day. For oral administration, the compositions are preferably provided in the form of tablets containing 0.01 to 1000 mg, particularly 0.01, 0.05, 0.1, 0.5, 1.0, 2.5, 3.0, 5.0, 6.0, 10.0, 15.0, 25.0, 50.0, 75, 100, 125, 150, 175, 180, 200, 225, and 500 milligrams of the active ingredient for the symptomatic adjustment of the dosage to the mammal to be treated.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

Upon improvement of a subject's condition, a maintenance dose of a compound, composition or combination of this invention may be administered, if necessary.

Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present invention will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration to a subject suffering from or susceptible to a disease or disorder.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the invention and a co-agent, are both administered to a patient simultaneously in the form of a single entity or dosage. The term “non-fixed combination” means that the active ingredients, e.g., a compound of the invention and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are another embodiment of the present invention.

### EXAMPLES

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. Definitions of variables in the structures in schemes herein are commensurate with those of corresponding positions in the formulae delineated herein.

### Example 1

### Synthesis

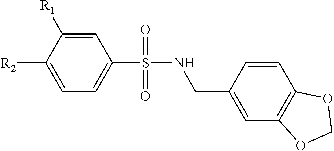

### Core Structure

| | R group | |
|---|---|---|
| SarCmpNumber | 3-position (R1) | 4-position (R2) |
| 1 | NC | CF₃ |
| 2 | NO₂ | CF₃ |
| 3 | CF₃ | NO₂ |
| 4 | CF₃ | NC |
| 5 | CN | CF₃ |
| 6 | CF₃ | CN |
| 7 | COCH₃ | CH₃ |
| 8 | COCH₂CH₃ | CH₃ |
| 9 | COCH₃ | NO₂ |
| 10 | COCH₂CH₃ | NO₂ |
| 11 | — | NHCOCH₃ |
| 12 | — | NHCOCH₂CH₃ |
| 13 | — | NHCOCH₂Br |
| 14 | — | NHCOCH₂CI |
| 15 | — | N(COCH₃)₂ |
| 16 | — | N(COCH₂CH₃)₂ |
| 17 | NHCOCH₂Br | — |
| 18 | NHCOCH₂CI | — |
| 19 | SCN | — |
| 20 | — | NHCOCF₃ |
| 21 | — | NHSO₂CH₃ |
| 22 | — | NHCOCH₂C(CH₃)₃ |
| 22A | | NHCOCH₂CH(CH₃)₂ |
| 23 | NCS | CF₃ |
| 24 | SCN | CF₃ |
| 25 | OCF₃ | COCH₃ |
| 26 | OCF₃ | COOH |
| 27 | OCF₃ | NO₂ |
| 28 | OCF₃ | COCH₃ |
| 29 | COCH₂CH₃ | OCF₃ |
| 29A | COCH₃ | OCF₃ |
| 30 | CONH₂ | OCF₃ |
| 31 | SO₂CH₃ | OCF₃ |
| 32 | CF₃ | COCH₃ |
| 33 | CF₃ | COCH₂CH₃ |
| 34 | CF₃ | COOH |
| 34A | CF₃ | CONH₂ |
| 35 | CF₃ | COCH₂CH₂CH₃ |
| 36 | OCF₃ | COCH₂CH₃ |

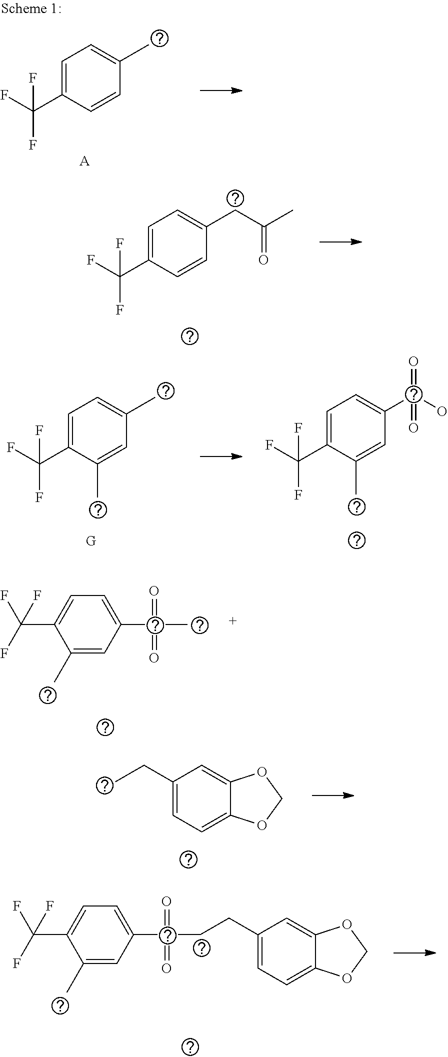

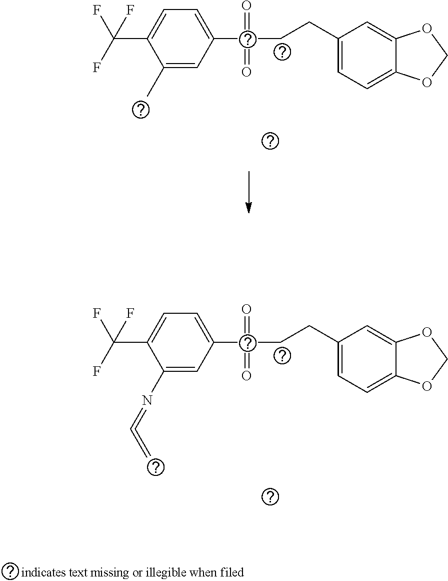

### Step 1: N-(4-(trifluoromethyl)phenyl)acetamide (B)

| | Chemicals/Reagents & | | | | | |
|---|---|---|---|---|---|---|
| Sr. No | Solvents | M. Wt | m.M | Eq. | Qty. | |
| 1. | 4-Aminobenzo trifluoride | 161 | 124.4 | 1 | 20 | |
| 2. | Acetic anhydride | 102 | 248.4 | 2 | 26 | g |
| 3. | Triethylamine | 101 | 310.5 | 2.5 | 40.2 | mL |
| 4. | DCM | | | | 200 | mL |

Reaction Time: 16 hour Reaction Temperature: 0° C. to rt.
Brief procedure: A mixture of 4-Amino benzotrifluoride, triethylamine was dissolved in DCM at 0° C. After 10 minutes acetic anhydride was added slowly. After complete addition, reaction mixture was stirred at rt for 16 hrs.
Work up: The reaction mixture was quenched with water and extracted with DCM. The organic layer was dried with Na₂SO₄ and evaporated the solvent.
Purification: used for next step without any further purifications
TLC system: 50% EtOAc in pet ether R_{f} value: 0.3
Nature: Off white solid. Yield: 15 gm (60%)

### Step 2: 3-nitro-4-(trifluoromethyl)aniline (C)

| | Chemicals/Reagents & | | | | | |
|---|---|---|---|---|---|---|
| Sr. No | Solvents | M. Wt | m.M | Eq. | Qty. | |
| 1. | Compound B | 203 | 123.5 | 1 | 25 | gm |
| 2. | KNO₂ | 101 | 147.78 | 1.2 | 14.94 | gm |
| 3. | H₂SO₄ | | | | 50 | mL |

### Reaction Time: 1 h Reaction Temperature: 0° C. to rt.

### Brief Procedure:

KNO₂ was added portion wise to sulfuric acid at 0° C. Then the nitration mixture was added to compound B in sulfuric acid at 0° C. Stirred the reaction mixture for 1 h at 0° C.

### Work Up:

The reaction mixture was poured into water and extracted with EtOAc. The combined organic layers washed with water followed by brine and dried over MgSO₄. The solvent was removed in vacuum to give the titled product which is used as it is in the next step.

TLC system: 30% EtOAc:Hexane R_{f} value: 0.2
Nature: yellow solid. Yield: 7.5 g (50%)

### Step 3: 3-nitro-4-(trifluoromethyl)benzene-1-sulfonyl chloride (1)

| | Chemicals/Reagents & | | | | | |
|---|---|---|---|---|---|---|
| Sr. No | Solvents | M. Wt | m.M | Eq. | Qty. | |
| 1. | Compound C | 206 | 24.27 | 1 | 5 | gm |
| 2. | NaNO₂ | 69 | 3.54 | 1.2 | 2.2 | gm |
| 3. | Acetic acid | — | — | — | 50 | mL |
| 4. | HC1 | — | — | — | 20 | mL |
| 5. | SO₂/ACOH/CUCl/H₂O | — | — | — | 40 | ml |

Reaction Time: 12 hour Reaction Temperature: 0° C. to rt.

### Brief Procedure:

Compound C suspension in glacial acetic acid was treated with a concentrated HCl. The resulting solution was then cooled approximately to 0° C. and treated drop wise with a solution of NaNO₂ in water. After 10 minutes the reaction mixture was added to a stirred solution of SO₂/ACOH/CUCl/H₂O (60 ml) (the preparation of the reagent from reported procedure (E. E. Gilbert, synthesis 1969, 1-10, p 6.) the reaction mixture was allowed to warm to room temperature and was stirred for 1 hour. The reaction was monitored by TLC.

### Work Up:

The reaction mixture was then poured into water and extracted with EtOAc. The combined organic layers were washed with water followed by brine and dried over MgSO₄. The solvent was removed under vacuum. to give the titled product which was used as crude in the next step.

TLC system: 30% EtOAc:Hexane R_{f} value: 0.6
Nature: Pale yellow solid. Yield: 2 g (29%)

### Step 4: N-(benzo[d][1,3]dioxol-5-ylmethyl)-3-nitro-4-(trifluoromethyl)benzenesulfonamide (comp2)

| | Chemicals/Reagents & | | | | | |
|---|---|---|---|---|---|---|
| S. No | Solvents | M. Wt | m.M | Eq. | Qty. | |
| 1. | Compound 1 | 286 | 6.62 | 1 | 2 | gm |
| 2. | Int-2 | 151 | 6.62 | 1.2 | 4 | gm |
| 3. | TEA | 101.1 | 13.24 | 1.3 | 3 | mL |
| 4. | DCM | — | — | — | 80 | mL |

### Brief Procedure:

Compound 1 was dissolved in DCM and TEA was added with stirring. The resulting solution was then cooled approximately to 0° c. and treated drop wise with a solution of Int-2. The reaction mixture is allowed to warm to room temperature and was stirred for 12 hour.

### Work Up:

The reaction mixture was then poured into water and extracted with DCM. The combined organic layers were washed with water followed by brine and dried over MgSO₄. The solvent was removed under reduced pressure

Purification: Purified through flash column chromatography using hexane and ethyl acetate as mobile phase.
TLC system: 50% EtOAc:Hexane R_{f} value: 0.3
Nature: White solid. Yield: 500 mg (20%)

### Step 4

### Preparation of N-(benzo[d][1,3]dioxol-5-ylmethyl)-3-isothiocyanato-4-(trifluoromethyl)benzenesulfonamide (compound 23)

| | Chemicals/Reagents & | | | | | |
|---|---|---|---|---|---|---|
| Sr. No | Solvents | M. Wt | m.M | Eq. | Qty. | |
| 1. | Int3 | 372 | 0.37 | 1 | 100 | mg |
| 2. | Triphosgene | 114 | 0.55 | 1.5 | 58 | mg |
| 3. | Triethylamine | 101 | 0.55 | 1.5 | 70 | mL |
| 4. | DCM | | | | 5 | mL |

Reaction Time: 1 hour. Reaction Temperature: 0° C.

### Brief Procedure:

The Int-3 was dissolved in DCM. Triphosgene was added at 0° C. and the reaction mixture was stirred for 1 hr at 0° C.

### Work Up:

The reaction mixture was then poured into water and extracted with DCM. The combined organic layers were washed with water followed by brine and dried over MgSO₄. The solvent was removed in vacuum.

Purification: Purified through flash column chromatography using hexane and ethyl acetate as mobile phase.
TLC system: 50% EtOAc:Hexane R_{f} value: 0.3
Nature: White solid. Yield: 20 mg

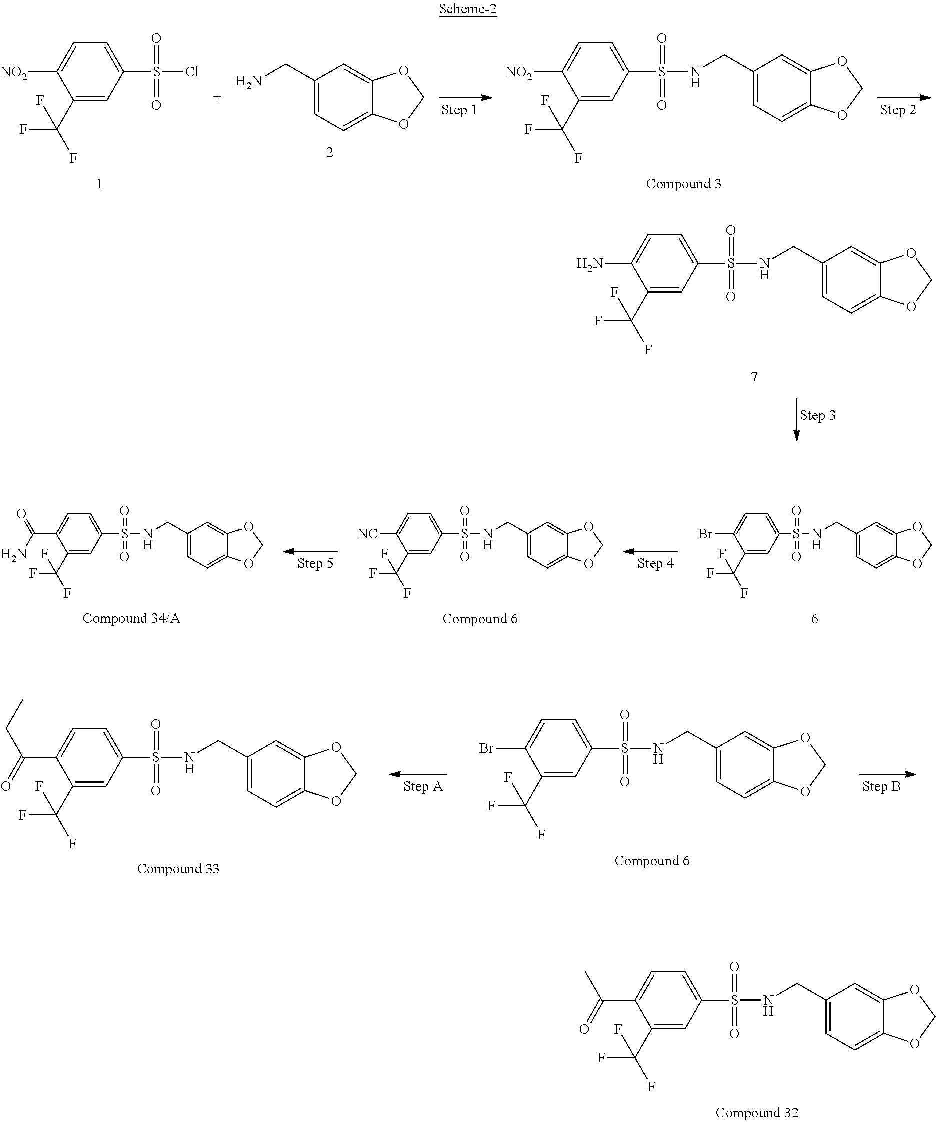

### Step 1: N-(benzo[d][1,3]dioxol-5-ylmethyl)-4-nitro-3-(trifluoromethyl)benzenesulfonamide (compound3)

| | Chemicals/Reagents & | | | | | |
|---|---|---|---|---|---|---|
| Sr. No | Solvents | M. Wt | m.M | Eq. | Qty. | |
| 1. | Compound 1 | 289 | 3.46 | 1 | 2 | gm |
| 2. | Compound 2 | 151 | 4.15 | 1.2 | 0.626 | g |
| 3. | TEA | 101.1 | 5.19 | 1.5 | 0.65 | mL |
| 4. | DCM | — | — | — | 80 | mL |

Reaction Time: 1 hour Reaction Temperature: 0° C. to rt.

### Brief Procedure:

Compound 1 was dissolved in DCM and TEA was added with stirring. The resulting solution was then cooled approximately to 0° c. and treated drop wise with a solution of 2. The reaction mixture was allowed to warm to room temperature and stirred for 12 hour.

### Work Up:

The reaction mixture was then poured into water and extracted with DCM. The combined organic layers were washed with water followed by brine and dried over MgSO₄. The solvent was removed under reduced pressure

Purification: Purified through flash column chromatography using hexane and ethyl acetate as mobile phase.
TLC system: 50% EtOAc:Hexane R_{f} value: 0.3
Nature: White solid. Yield: 2 g (55%)

### Step 2. 4-amino-N-(benzo[d][1,3]dioxol-5-ylmethyl)-3-(trifluoromethyl)benzenesulfonamide (3)

| | Chemicals/Reagents & | | | | | |
|---|---|---|---|---|---|---|
| Sr. No | Solvents | M. Wt | m.M | Eq. | Qty. | |
| 1. | Compound 3 | 402 | 4.97 | 1 | 2 | g |
| 2. | EtOH:THF:H2O (4:2:1) | | | | 35 | mL |
| 3. | Iron powder (Fe) | 56 | 19.99 | 4 | 1.13 | g |
| 4. | Ammonium chloride | 53.5 | 17.39 | 3.5 | 0.93 | g |

Reaction Time: 2 hour Reaction Temperature: 80° C.

### Brief Procedure:

Compound 3 was taken in EtOH:THF:H₂O (4:2:1) and then was added iron powder (Fe) and ammonium chloride at RT (25° C.). The mixture was refluxed for 2 hours. Reaction was monitored by TLC.

Work up: The reaction mixture was concentrated to get crude compound. Crude compound was taken in EtOAc, washed with water and brine solution. EtOAc layer was dried over anhydrous Na₂SO₄ and the concentrated under reduced pressure.
Purification: crude compound purified by flash column chromatography using EtOAc/Hexane as mobile phase.
TLC system: 40% EtOAc:Hexane R_{f} value: 0.2
Nature: Light brown solid. Yield: 1.2 g (65.93%)

### Step 3: N-(benzo[d][1,3]dioxol-5-ylmethyl)-4-bromo-3-(trifluoromethyl)benzenesulfonamide (compound 6)

| | Chemicals/Reagents & | | | | | |
|---|---|---|---|---|---|---|
| Sr. No | Solvents | M. Wt | m.M | Eq. | Qty. | |
| 1. | Compound 7 | 374 | 2.67 | 1 | 1 | |
| 2. | NaNO₂ | 69 | 3.20 | 1.2 | 220 | mg |
| 3. | Acetic acid | — | — | — | 10 | mL |
| 4. | HCl | — | — | — | 10 | mL |
| 5. | H₂O | | | | 20 | mL |
| 6. | CuBr | 145 | 4.00 | 1.5 | 580 | mg |
| 7. | HBr in ACOH | — | — | — | 40 | mL |

Reaction Time: 2 hour Reaction Temperature: 0° C. to rt.

### Procedure Brief:

Compound 7 suspension in glacial acetic acid was treated with a concentrated HCl and water. The resulting solution was then cooled approximately to 0° C. and was treated drop wise with a solution of NaNO₂ in water. After 30 minutes, the reaction mixture was added to stirred solution of CuBr in HBr, acetic acid (40 ml). The reaction mixture was allowed to warm to room temperature and stirred for 1 hour. The reaction was monitored by TLC.

### Work Up:

The reaction mixture was then poured into water and extracted with EtOAc. The combined organic layers were washed with water followed by brine and dried over MgSO₄. The solvent was removed in vacuum to give the titled product which was used crude in the next step.

TLC system: 40% EtOAc:Hexane R₁ value: 0.6
Nature: Off white solid. Yield: 600 mg (53%)

### Step B: Preparation of 4-acetyl-N-(benzo[d][1,3]dioxol-5-ylmethyl)-3-(trifluoromethyl)benzenesulfonamide compound (32)

| | Chemicals/Reagents & | | | | | |
|---|---|---|---|---|---|---|
| Sr. No | Solvents | M. Wt | m.M | Eq. | Qty. | |
| 1. | Int-6 | 435 | 0.229 | 1 | 100 | mg |
| 2. | tributyl(1-ethoxyvinyl) tin | 361.4 | 0.459 | 2 | 165 | mg |
| 3. | Tetrakistriphenylphosphine- | 701.81 | 0.011 | 0.05 | 8 | mg |
| | palladium | | | | | |
| 4. | 1,4-dioxane | — | — | — | 20 | mL |

Reaction Time: 16 hour Reaction Temperature: 85° C.

### Brief Procedure:

Int-6, tributyl (1-ehoxyvinyl) tin and tetrakistriphenylphosphinepalladium were added to dioxin in a seal tube. The reaction mixture was stirred overnight at 85° C. then diluted with ethyl acetate, washed with sat. NaHCO₃ and brine, dried over Na₂SO₄ and evaporated. The residue was re-dissolved in THF (10 ml), 1N HCl was added and the mixture was stirred for 18 h. The solvent was removed in vacuum.

Purification: Purified through flash column chromatography using hexane and ethyl acetate as mobile phase.
TLC system: 50% EtOAc:Hexane value: 0.3
Nature: Off white solid. Yield: 25 mg

### Step A: Preparation of N-(benzo[d][1,3]dioxol-5-ylmethyl)-4-propionyl-3-(trifluoromethyl)benzenesulfonamide (compound 33)

| | Chemicals/Reagents & | | | | | |
|---|---|---|---|---|---|---|
| Sr. No | Solvents | M. Wt | m.M | Eq. | Qty. | |
| 1. | Int-6 | 435 | 0.229 | 1 | 100 | mg |
| 2. | tributyl(1-ethoxyprop-1-enyl) | 361.4 | 0.458 | 2 | 165 | mg |
| | tin | | | | | |
| 3. | Tetrakistriphenylphosphine- | 701.81 | 0.027 | 0.05 | 8 | mg |
| | palladium | | | | | |
| 4. | 1,4-dioxane | — | — | — | 20 | mL |

Reaction Time: 16 hour. Reaction Temperature: 85° C.

### Brief Procedure:

Int-6, tributyl (1-ehoxyprop-1-enyl) tin and tetrakistriphenylphosphinepalladium were added to dioxin in a seal tube. The reaction mixture was stirred overnight at 85° C. then diluted with ethyl acetate, washed with sat. NaHCO₃ and brine, dried over Na₂SO₄ and evaporated. The residue was re-dissolved in THF (10 ml), 1N HCl was added and the mixture was stirred for 18 h. The solvent was removed in vacuum.

Purification: Purified through flash column chromatography using hexane and ethyl acetate as mobile phase.
TLC system: 50% EtOAc:Hexane R_{f} value: 0.3
Nature: White solid. Yield: 22 mg

### Step 4: preparation of N-(benzo[d][1,3]dioxol-5-ylmethyl)-4-isocyano-3-(trifluoromethyl)benzenesulfonamide (compound 6)

| Sr. No | Chemicals/Reagents & Solvents | M. Wt | m.M | Eq. | Qty. | |
|---|---|---|---|---|---|---|
| 1. | Int-6 | 435 | 0.229 | 1 | 100 | mg |
| 2. | CuCN | 89.56 | 1.145 | 5 | 102 | mg |
| 3. | NMP | — | — | — | 20 | mL |

Reaction Time: 16 hour. Reaction Temperature: 180° C.

### Brief Procedure:

Int-6 and CuCN were added to NMP (20 ml) in a seal tube. The reaction mixture was stirred overnight at 180° C. The reaction was monitored by TLC, then diluted with ethyl acetate, washed with water and brine, dried over Na₂SO₄ and evaporated.

### Purification:

Purified by flash column chromatography using hexane and ethyl acetate as mobile phase.

TLC system: 50% EtOAc:Hexane R_{f} value: 0.3
Nature: Brown solid. Yield: 60 mg (69%)

### Step 5: preparation of 4-(N-(benzo[d][1,3]dioxol-5-ylmethyl)sulfamoyl)-2-(trifluoromethyl)benzamide (compound 34/A)

| Sr. No | Chemicals/Reagents & Solvents | M. Wt | m.M | Eq. | Qty. | |
|---|---|---|---|---|---|---|
| 1. | Compound-6 | 384 | 0.130 | 1 | 50 | mg |
| 2. | NaOH | 40 | 0.651 | 5 | 26 | mg |
| 3. | Water | — | — | — | 3 | mL |
| 4. | Methanol | — | — | — | 3 | mL |

Reaction Time: 18 hour. Reaction Temperature: 100° C.

### Brief Procedure:

Compound 6 and NaOH were added to a mixture of methanol and water (20 ml) in an RBF. The reaction mixture was stirred overnight at 100° C. The reaction was monitored by TLC, then diluted with ethyl acetate, washed with water and brine, dried over Na₂SO₄ and evaporated.
Purification: Purified by base-acid techniques.
TLC system: 10% MeOH: DCM R_{f} value: 0.4
Nature: Off white solid. Yield: 25 mg (50%)

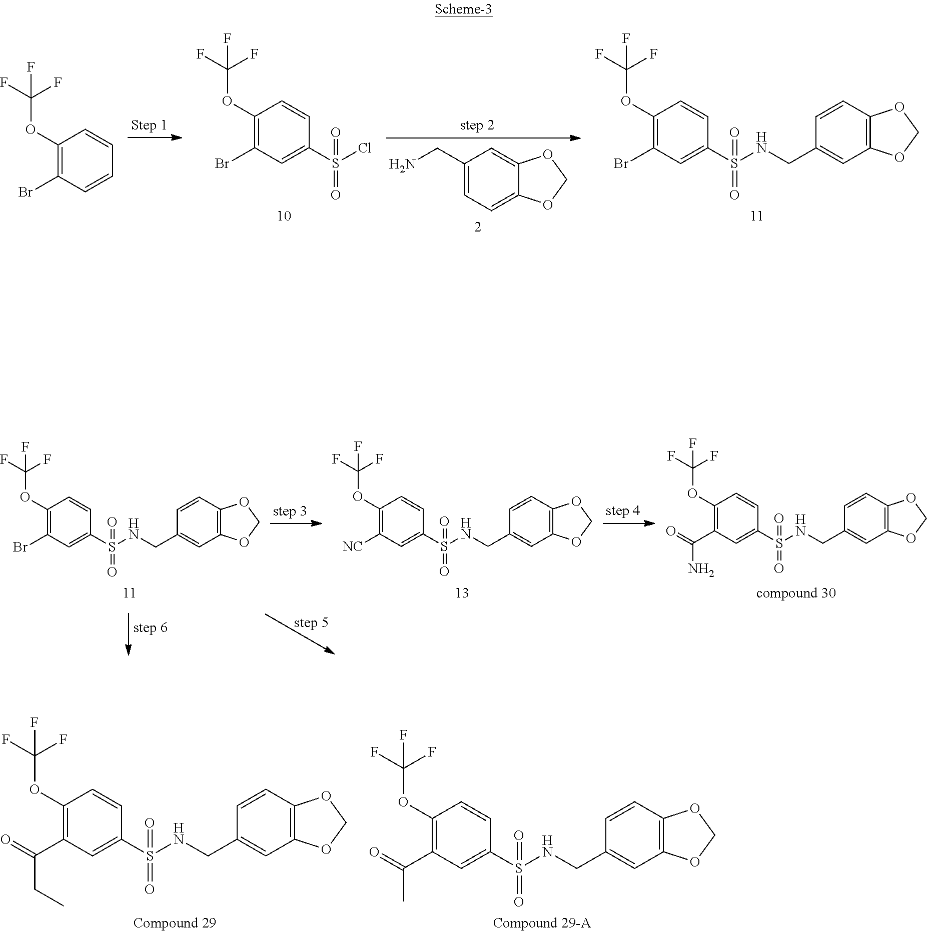

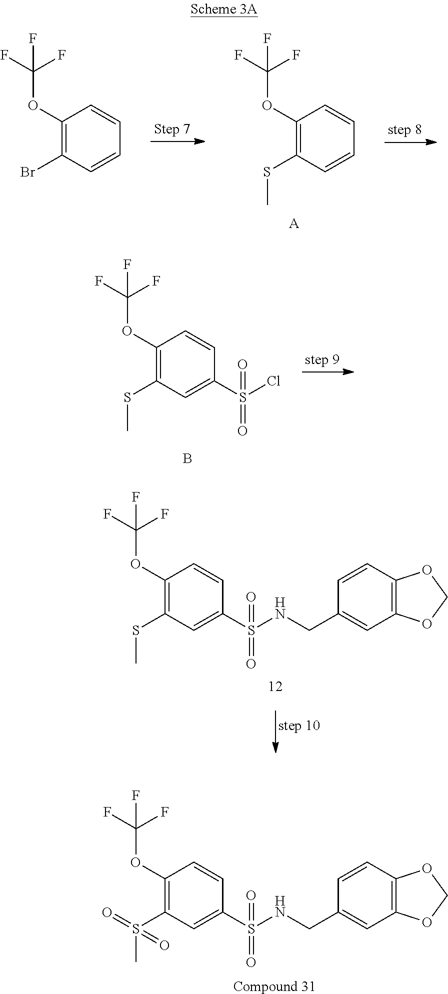

### Step 1

### Preparation of 3-bromo-4-(trifluoromethoxy)benzene-1-sulfonyl chloride (10)

| Sr. | | | | | | |
|---|---|---|---|---|---|---|
| No | Chemicals/Reagents & Solvents | M.Wt | m.M | Eq. | Qty. | |
| 5. | 1-bromo-2-(trifluoromethoxy) | 241 | 8.298 | 1 | 2 | g |
| | bezene | | | | | |
| 6. | Chlorosulfonoic acid(d: 1.753) | 116.52 | 58.09 | 7 | 6.76 | g |
| 7. | DCM | — | — | | 80 | mL |

Reaction Time: 3 hour Reaction Temperature: rt.
Brief procedure: 1-bromo-2-(trifluoromethoxy)benzene was dissolved in DCM and under cooling condition (0° C.). chlorosulfonic acid was added. The reaction mixture then allowed to stir at rt for 3 hr.
Work up: The reaction mixture was poured into ice cold water, extracted with ethyle acetate (25 mL×3), organic layer was evaporated under reduced pressure and subjected to column chromatography using 10% EtOAc:Hexane
TLC system: 5% EtOAc:Hexane R_{f} value: 0.6
Nature: Brown liquid. Yield: 1.2 (44.4%)
Analytical Data: The product is confirmed by ¹HNMR and LCMS.

### Step 2

### preparation of N-(benzo[d][1,3]dioxol-5-ylmethyl)-3-bromo-4-(trifluoromethoxy)benzenesulfonamide (11)

| Sr. | Chemicals/ | | | | | |
|---|---|---|---|---|---|---|
| No | Reagents & Solvents | M.Wt | m.M | Eq. | Qty. | |
| 8. | Intermediate 10 | 337.8 | 0.59 | 1 | 200 | mg |
| 9. | Reagent B | 151.65 | 0.59 | 1 | 87.78 | mg |
| 10. | TEA | 101.19 | 1.77 | 3 | 179.10 | mg |
| 11. | DCM | — | — | — | 10 | mL |

Reaction Time: 3 hour Reaction Temperature: 0° C. to rt.

### Brief Procedure:

Compound 10 & Reagent 2 were dissolved in DCM, TEA was added under cooled condition (at 0° C.) and allowed to stir at rt for 3 hr. The reaction monitored by TLC.

### Work Up:

The reaction mixture was poured into water and extracted with EtOAc (25 mL×3). The combined organic layer was washed with water, brine and dried over MgSO₄. The solvent was removed in vacuum and residue was subjected to column chromatography where expected spot eluted at 15% EtOAc:Hexane.

TLC system: 30% EtOAc:Hexane R_{f} value: 0.6
Nature: White colored solid. Yield: 200 mg (76.92%)
Analytical Data: The product is confirmed by ¹HNMR and LCMS

### Steps 5

### Preparation of N-(benzo[d][1,3]dioxol-5-ylmethyl)-3-cyano-4-(trifluoromethoxy)benzenesulfonamide (13)

| Sr. | | | | | | |
|---|---|---|---|---|---|---|
| No | Chemicals/Reagents & Solvents | M.Wt | m.M | Eq. | Qty. | |
| 1. | Intermediate 11 | 454.1 | 2.20 | 1 | l | g |
| 2. | CuCN | 89.56 | 11.0 | 5 | 986.1 | mg |
| 3. | NMP | — | — | — | 20 | mL |

Reaction Time: 16 hour. Reaction Temperature: 150° C.

### Brief Procedure:

Intermediate 11 and CuCN were added to NMP in a seal tube. The reaction mixture was stirred overnight at 150° C. The reaction was monitored by TLC, then diluted with ethyl acetate, washed with water and brine, dried over Na₂SO₄ and evaporated.

### Purification:

Purified by flash column chromatography. Using hexane and ethyl acetate as mobile phase.

TLC system: 30% EtOAc:Hexane R_{f} value: 0.6
Nature: White solid. Yield: 500 mg (56.81%)
Analytical Data: The product is confirmed by ¹HNMR and LCMS

### Step 7

### Preparation of methyl(2-(trifluoromethoxy)phenyl)sulfane (A)

| Sr. | | | | | | |
|---|---|---|---|---|---|---|
| No | Chemicals/Reagents & Solvents | M.Wt | m.M | Eq. | Qty. | |
| 5. | 1-bromo-2-(trifluoromethoxy) | 241 | 4.14 | 1 | 1 | g |
| | bezene | | | | | |
| 6. | Sodium methane thiolate | 70.09 | 4.97 | 1.2 | 348 | mg |
| 7. | DMA | — | | — | 10 | mL |

### Reaction Time: 3 hr Reaction Temperature: 80° C.

### Brief Procedure:

1-bromo-2-(trifluoromethoxy)benzene & Sodium methane thiolate were dissolved in DMA and allowed to stir at 80° C. for 3 hr.

### Work Up:

The reaction mixture is then poured into water and extracted with DCM (25 mL×3). The combined organic layer was washed with water, brine and dried over MgSO₄. The solvent was removed under reduced pressure.

Purification: Purified through flash column chromatography using hexane and ethyl acetate as mobile phase.
TLC system: 5% EtOAc:Hexane R_{f} value: 0.6
Nature: yellow color liquid. Yield: 200 mg (23.25%)
Analytical Data: The product is confirmed by ¹HNMR and LCMS

### Step 8

### Preparation of 3-(methylthio)-4-(trifluoromethoxy)benzene-1-sulfonyl chloride (B)

| Sr. | | | | | | |
|---|---|---|---|---|---|---|
| No | Chemicals/Reagents & Solvents | M.Wt | m.M | Eq. | Qty. | |
| 5. | Intermediate A | 208 | 0.96 | 1 | 200 | mg |
| 6. | Chlorosulfonoic acid(d: 1.753) | 116.52 | 6.73 | 7 | 784.26 | mg |
| 7. | DCM | — | — | — | 10 | mL |

Reaction Time: 3 hour. Reaction Temperature: rt

### Brief Procedure:

Compound A was dissolved in DCM and under cooled condition (0° C.) chlorosulfonic acid were added. The reaction mixture was allowed to stir at rt for 3 hr.

Work up: The reaction mixture was poured into ice cold water and extracted with ethyl acetate (25 mL×3). Evaporation of the solvent under reduced pressure and column chromatography using 10% EtOAC: Hexane gave the desired product.
TLC system: 10% EtOAc:Hexane 19 value: 0.4
Nature: Light brown color liquid. Yield: 300 mg
Analytical Data: The product is confirmed by ¹HNMR and LCMS

### Step 9

### Preparation of N-(benzo[d][1,3]dioxol-5-ylmethyl)-3-(methylthio)-4-(trifluoromethoxy)benzenesulfonamide (12)

| Sr. | | | | | | |
|---|---|---|---|---|---|---|
| No | Chemicals/Reagents & Solvents | M.Wt | m.M | Eq. | Qty. | |
| 5. | Intermediate B(crude) | 305.9 | 0.98 | 1 | 300 | mg |
| 6. | Reagent 2 | 151.65 | 0.98 | 1 | 146.75 | mg |
| 7. | TEA | 101.19 | 2.94 | 3 | 297.7 | mg |
| 8. | DCM | — | — | — | 10 | mL |

Reaction Time: 3 hour. Reaction Temperature: rt.

### Brief Procedure:

Intermediate B & Reagent 2 were dissolved in DCM and to this was added TEA under cooled condition (at 0° C.). The reaction mixture was allowed to stir at rt for 3 hr with monitoring by TLC.

### Work Up:

The reaction mixture was poured into water and extracted with EtOAc (25 mL×3). The combined organic layer was washed with water brine and dried over MgSO₄. The solvent was removed under reduced pressure and residue was subjected to column chromatography where expected spot eluted at 15% EtOAc:Hexane.

TLC system: 30% EtOAc:Hexane R_{f} value: 0.5
Nature: white solid 150 mg
Analytical Data: The product is confirmed by ¹HNMR and LCMS

### Step 10

### Preparation of N-(benzo[d][1,3]dioxol-5-ylmethyl)-3-(methylsulfonyl)-4-(trifluoromethoxy)benzenesulfonamide (compound 31)

| Sr. | Chemicals/Reagents & | | | | | |
|---|---|---|---|---|---|---|
| No | Solvents | M.Wt | m.M | Eq. | Qty. | |
| 4. | Intermediate 12 | 421 | 0.35 | 1 | 150 | mg |
| 5. | Oxone | 614.78 | 1.068 | 3 | 657.1 | mg |
| 6. | CH₃CN | — | — | — | 20 | ml |
| 7. | H₂O | — | — | — | 5 | ml |

### Reaction Time: 24 hr Reaction Temperature: rt

### Brief Procedure:

Intermediate 12 was dissolved in CH₃CN and water, oxone were added to reaction mixture then allowed to stir at rt for 24 hr. Reaction was monitored by TLC, then diluted with ethyl acetate, washed with water, brine, dried over Na₂SO₄ and evaporated.

### Purification:

Purified by flash column chromatography using hexane and ethyl acetate as a mobile phase.

TLC system: 50% EtOAC: Hexane R_{f} value: 0.2
Nature: White solid. Yield: 55 mg (34.16%)
Analytical Data: The product is confirmed by ¹HNMR and LCMS

### Step 5

### Preparation of 3-acetyl-N-(benzo[d][1,3]dioxol-5-ylmethyl)-4-(trifluoromethoxy)benzenesulfonamide (compound 29-A)

| Sr. | Chemicals/Reagents & | | | | | |
|---|---|---|---|---|---|---|
| No | Solvents | M.Wt | m.M | Eq. | Qty. | |
| 5. | Intermediate 11 | 452 | 0.66 | 1 | 300 | mg |
| 6. | 1-ethoxy vinyl tri n- | 361.1 | 40.99 | 1.5 | 359.5 | mg |
| | butyl tin | | | | | |
| 7. | Pd(PPh₃)₂Cl₂ | 701.81 | 0.039 | 0.06 | 27.79 | mg |
| 8. | DMF | — | — | — | 10 | mL |

Reaction Time: 2 hour. Reaction Temperature: 150° C.

### Brief Procedure:

Intermediate 11 was dissolved in DMF, degassed with N₂ gas (15 min) then added “Pd(PPh₃)₂Cl₂” again degassed with N₂ gas and added“1-ethoxy vinyltrin-butyltin”, The reaction mixture then was allowed to stir and irradiated in microwave at 150° c. for 2 h. monitored by TLC, new spots found at Rf 0.8 & 0.9, total reaction mixture treated with sat. KF solution and filtered through celite bed, to the filtrate given EtOAc extractions (25 ml×3). The organic layer was stirred with 3N HCl (100 ml) at rt for overnight, dried with Na₂SO₄ and evaporated.

### Purification:

Purified by preparative HPLC

TLC system: 10% EtOAC:Hexane R_{f} value: 0.4
Nature: White solid. Yield: 140 mg (50.58%)
Analytical Data: The product is confirmed by ¹HNMR and LCMS

### Step 6: Preparation of N-(benzo[d][1,3]dioxol-5-ylmethyl)-3-propionyl-4-(trifluoromethoxy)benzenesulfonamide (compound 29)

| S. | Chemicals/Reagents & | | | | | |
|---|---|---|---|---|---|---|
| No | Solvents | M.Wt | m.M | Eq. | Qty. | |
| 1. | Compound 11 | 452.1 | 0.55 | 1 | 250 | mg |
| 2. | tributyl(1-ethoxyprop-1- | 375.4 | 1.1 | 2 | 412 | mg |
| | enyl) tin | | | | | |
| 3. | Tetrakistriphenyl- | 1155.1 | 0.027 | 0.05 | 30 | mg |
| | phosphinepalladium | | | | | |
| 4. | 1,4-dioxane | — | — | — | 20 | ml |

Reaction Time: 16 hour. Reaction Temperature: 85° C.

### Brief Procedure:

Compound 11, tributyl (1-ehoxyprop-1-enyl) tin and tetrakistriphenylphosphinepalladium were added to 1,4-dioxane in a seal tube. The reaction mixture was stirred overnight at 85° C. then diluted with ethyl acetate, washed with sat. NaHCO₃, brine, dried over Na₂SO and evaporated. The residue was re-dissolved in THF (10 mL), 1N HCl was added and the mixture was stirred for 18 h. The solvent was removed in vacuum.

### Purification:

Purified by flash column chromatography using hexane and ethyl acetate as mobile phase.

TLC system: 50% EtOAc:Hexane R_{f} value: 0.3
Nature: White solid. Yield: 30 mg

### Step 4

### Preparation of 5-(N-(benzo[d][1,3]dioxol-5-ylmethyl)sulfamoyl)-2-(trifluoromethoxy)benzamide (compound 30)

| Sr. | | | | | | |
|---|---|---|---|---|---|---|
| No | Chemicals/Reagents & Solvents | M.Wt | m.M | Eq. | Qty. | |
| 1. | Intermediate 13 | 400 | 0.75 | 1 | 300 | mg |
| 2. | NaOH | 40 | 3.75 | 5 | 150 | mg |
| 3. | McOH | — | — | — | 10 | mL |
| 4. | H₂O | — | — | — | 5 | mL |

Reaction Time: 3 hour. Reaction Temperature: 150° C.

### Brief Procedure:

Intermediate 13 was dissolved in McOH and H₂O, NaOH was added to reaction mixture under cooled condition (at 0° C.) allowed to stir at 50° C. for 3 hr. The progress of reaction monitored by TLC.

### Work Up:

The reaction mixture was poured into water (20 mL) and extracted with EtOAc (15 mL×3). The combined organic layers washed with water followed by brine and dried over MgSO₄. The solvent was removed under reduced pressure.

### Purification:

Purified by preparative HPLC

TLC system: 30% EtOAc:Hexane/4 value: 0.4
Nature: White solid. Yield: 80 mg (25.51%)
Analytical Data: The product is confirmed by ¹HNMR and LCMS

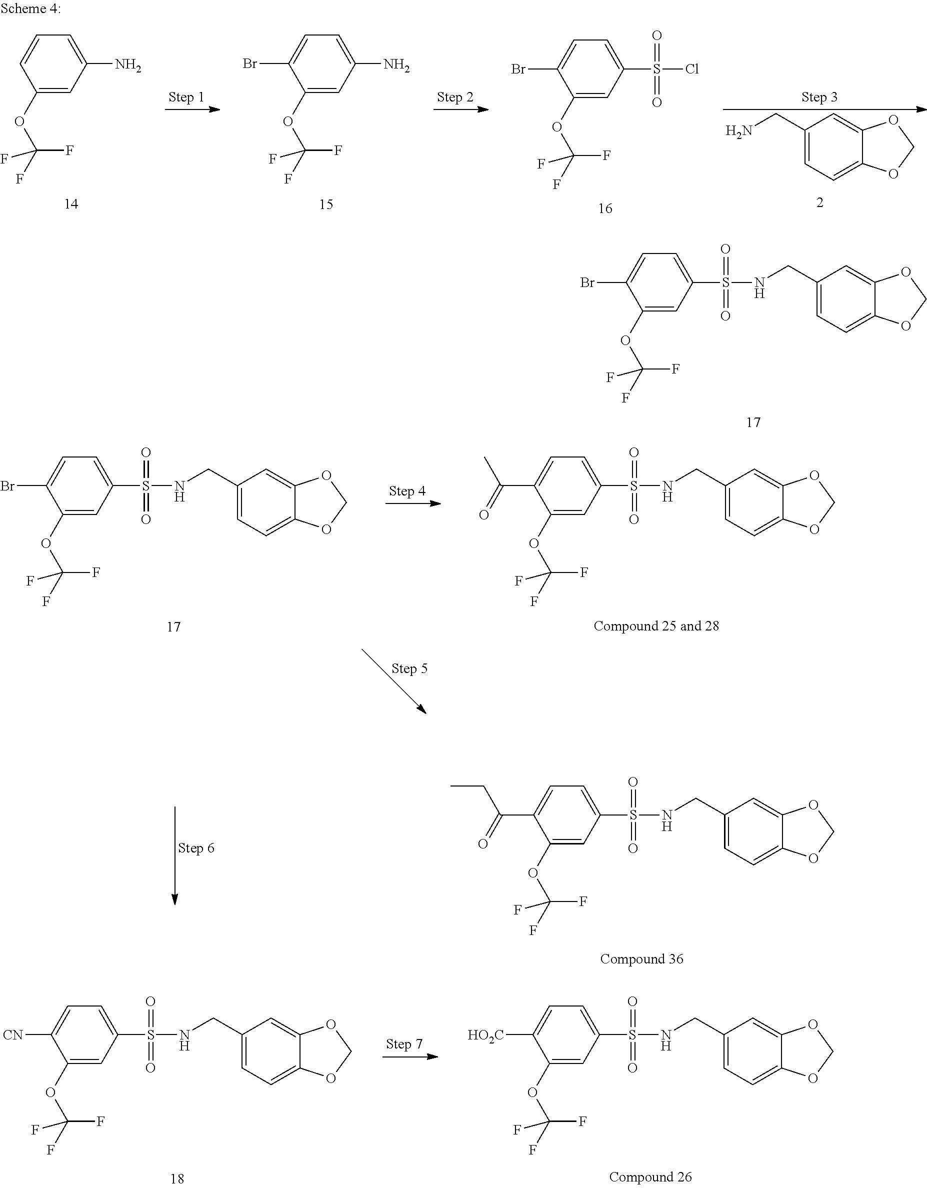

### Step 1

### Preparation of 4-bromo-3-(trifluoromethoxy)aniline (15)

| Sr. | | | | | | |
|---|---|---|---|---|---|---|
| No | Chemicals/Reagents & Solvents | M.Wt | m.M | Eq. | Qty. | |
| 1. | 3-(trifluoromethoxy)aniline | 177.13 | 12.9 | 1 | 2.3 | g |
| 2. | NBS | 178 | 15.6 | 1.2 | 2.6 | g |
| 3. | Silica gel | — | — | 30% | 0.69 | g |
| 4. | DCM | | | | 60 | mL |

### Reaction Time: 1 h Reaction Temperature: −5° C. to rt.

### Brief Procedure:

A mixture of 3-(trifluoromethoxy) aniline, silica gel and NBS in DCM was stirred for 1 h. Reaction was monitored by TLC.

Work up: The reaction mixture was filtered, diluted with water and extracted in DCM. Organic layer was evaporated under reduced pressure.
Purification: Purified through flash column chromatography using hexane and ethyl acetate as mobile phase.
TLC system: 10% EtOAc:Hexane R_{f} value: 0.4
Nature: Brown liquid.
Analytical Data: The product is confirmed by ¹HNMR and LCMS

### Step 2

### Preparation of 4-Bromo-3-9-trifluoromethoxy)benzene sulfonamide (16)

| Sr. | | | | | | |
|---|---|---|---|---|---|---|
| No | Chemicals/Reagents & Solvents | M.Wt | m.M | Eq. | Qty. | |
| 1. | Compound 15 | 254 | 2.95 | 1 | 700 | mg |
| 2. | NaNO₂ | 69 | 3.54 | 1.2 | 250 | mg |
| 3. | Acetic acid | — | — | — | 27.5 | mL |
| 4. | HCl | — | — | — | 7.5 | mL |
| 5. | SO₂/ACOH/CUCl₂/H₂O | — | — | — | 60 | mL |

### Reaction Time: 1 h Reaction Temperature: 0° C. to rt.

### Brief Procedure:

The suspension of compound 15 in glacial acetic acid was treated with a concentrated HCl. The resulting solution was cooled approximately to 0° C. and treated drop wise with a solution of NaNO₂ in water. After 10 minutes, the reaction mixture was added to a stir solution of SO₂/ACOH/CuCl₂/H₂O (60 ml) (the preparation of the reagent from reported procedure E. E. Gilbert, synthesis 1969, 1-10, p 6.) the reaction mixture was allowed to warm to room temperature and stirred for 1 hour. The reaction was monitored by TLC.

### Work Up:

The reaction mixture was then poured into water and extracted with EtOAc. The combined organic layers were washed with water brine and dried over MgSO₄. The solvent was removed under reduced pressure to give the titled product which was used crude in the next step.

TLC system: 30% EtOAC: Hexane R_{f} value: 0.2
Nature: yellow liquid. Yield: 600 mg (crude)
Analytical Data: The product is confirmed by ¹HNMR and LCMS

### Step 3

### Preparation of N-(benzo[d][1,3]dioxol-5-ylmethyl)-4-bromo-3-(trifluoromethoxy)benzenesulfonamide (17)

| Sr. | | | | | | |
|---|---|---|---|---|---|---|
| No | Chemicals/Reagents & Solvents | M.Wt | m.M | Eq. | Qty. | |
| 1. | Compound 16 | 339.5 | 1.76 | 1 | 600 | mg |
| 2. | Compound 2 | 69 | 1.94 | 1.2 | 300 | mg |
| 3. | TEA | 101.1 | 2.3 | 1.3 | 230 | mg |
| 4. | DCM | — | — | — | 80 | mL |

Reaction Time: 12 hour Reaction Temperature: 0° C. to rt.

### Brief Procedure:

Compound 16 was dissolved in DCM and TEA was added. The resulting solution was then cooled approximately to 0° C. and treated drop wise with a solution of compound 2 in DCM. The reaction mixture was allowed to warm to room temperature and stirred for 12 hours.

### Work Up:

The reaction mixture was poured into water and extracted with DCM. The combined organic layers washed with water, brine and dried over MgSO₄. The solvent was removed under reduced pressure.

Purification: Purified through flash column chromatography using hexane and ethyl acetate as mobile phase.
TLC system: 50% EtOAc:Hexane R_{f} value: 0.3
Nature: White solid. Yield: 400 mg (50%)
Analytical Data: The product is confirmed by ¹HNMR and LCMS

### Step 4

### Preparation of 4-acetyl-N-(benzo[d][1,3]dioxol-5-ylmethyl)-3-(trifluoromethoxy)benzenesulfonamide (compound 25)

| Sr. | Chemicals/Reagents & | | | | | |
|---|---|---|---|---|---|---|
| No | Solvents | M.Wt | m.M | Eq. | Qty. | |
| 1. | Compound 17 | 454.1 | 0.55 | 1 | 250 | mg |
| 2. | tributyl(1-ethoxyvinyl) | 361.4 | 1.1 | 2 | 400 | mg |
| | tin | | | | | |
| 3. | Tetralcistriphenyl- | 701.81 | 0.027 | 0.05 | 20 | mg |
| | phosphinepalladium | | | | | |
| 4. | 1,4-dioxane | — | — | — | 20 | mL |

### Reaction Time: 16 h Reaction Temperature: 85° C.

### Brief Procedure:

Compound 17, tributyl (1-ehoxyvinyl) tin and Tetrakistriphenylphosphinepalladium were added to dioxane in a seal tube. The reaction mixture was stirred overnight at 85° C., then diluted with ethyl acetate, washed with sat. NaHCO₃, brine, dried over Na₂SO and evaporated. The residue is dissolved in THF (10 mL), 1N HCl was added and the mixture was stirred for 18 h. The solvent was removed under reduced pressure.

Purification: Purified through flash column chromatography using hexane and ethyl acetate as mobile phase.
TLC system: 50% EtOAc:Hexane R_{f} value: 0.3
Nature: yellow solid. Yield: 15 mg
Analytical Data: The product is confirmed by ¹HNMR and LCMS

### Step 5

### Preparation of N-(benzo[d][1,3]dioxol-5-ylmethyl)-4-propionyl-3-(trifluoromethoxy)benzenesulfonamide (compound 36)

| Sr. | Chemicals/Reagents & | | | | | |
|---|---|---|---|---|---|---|
| No | Solvents | M.Wt | m.M | Eq. | Qty. | |
| 1. | Compound 17 | 454.1 | 0.55 | 1 | 250 | mg |
| 2. | tributyl(1-ethoxyprop-1- | 361.4 | 1.1 | 2 | 400 | mg |
| | enyl) tin | | | | | |
| 3. | Tetrakistriphenyl- | 701.81 | 0.027 | 0.05 | 20 | mg |
| | phosphinepalladium | | | | | |
| 4. | 1,4-dioxane | — | — | — | 20 | mL |

### Reaction Time: 16 h Reaction Temperature: 85° C.

### Brief Procedure:

Compound 17, tributyl (1-ehoxyprop-1-enyl) tin and Tetrakistriphenylphosphinepalladium were added to dioxane in a seal tube. The reaction mixture was stirred overnight at 85° C., then diluted with ethyl acetate, washed with sat. NaHCO₃, brine, dried over Na₂SO and evaporated. The residue was redissolved in THF (10 mL), 1N HCl was added and the mixture stirred for 18 h. The solvent was removed under reduced pressure

### Purification:

Purified by flash column chromatography using hexane and ethyl acetate as mobile phase.

TLC system: 50% EtOAC: Hexane R_{f} value: 0.3
Nature: yellow solid. Yield: 12 mg
Analytical Data: The product is confirmed by ¹HNMR and LCMS

### Step 6

### preparation of N-(benzo[d][1,3]dioxol-5-ylmethyl)-4-cyano-3-(trifluoromethoxy)benzenesulfonamide (18)

| Sr.No | Chemicals/Reagents &Solvents | M.Wt | m.M | Eq. | Qty. | |
|---|---|---|---|---|---|---|
| 1. | Compound 17 | 454.1 | 0.88 | 1 | 400 | mg |
| 2. | CuCN | 89.56 | 4.40 | 5 | 400 | mg |
| 3. | NMP | — | — | — | 20 | mL |

Reaction Time: 16 h. Reaction Temperature: 150° C.

### Brief Procedure:

Compound 17 and CuCN were added to NMP (20 mL) in a seal tube. The reaction mixture was stirred overnight at 150° C. The reaction was monitored by TLC, then diluted with ethyl acetate, washed with water, brine, dried over Na₂SO₄ and evaporated.

### Purification:

Purified by flash column chromatography using hexane and ethyl acetate as mobile phase.

TLC system: 50% EtOAc:Hexane R_{f} value: 0.3
Nature: Brown solid. Yield: 240 mg (68%)
Analytical Data: The product is confirmed by ¹HNMR and LCMS

### Step 7

### Preparation 4-(N-(benzo[d][1,3]dioxol-5-ylmethyl)sulfamoyl)-2-(trifluoromethoxy)benzoic acid of (compound 26)

| Sr.No | Chemicals/Reagents &Solvents | M.Wt | m.M | Eq. | Qty. | |
|---|---|---|---|---|---|---|
| 1. | Compound 18 | 400.1 | 0.59 | 1 | 240 | mg |
| 2. | NaOH | 40 | 2.9 | 5 | 119 | mg |
| 3. | Water | — | — | — | 10 | mL |
| 4. | Methanol | — | — | — | 10 | mL |

### Reaction Time: 18 h Reaction Temperature: 100° C.

### Brief Procedure:

Compound 18 and NaOH were added to a mixture of methanol and water (20 mL). The reaction mixture was stirred overnight at 100° C. The reaction was monitored by TLC, then diluted with ethyl acetate, washed with water and brine, dried over Na₂SO₄ and evaporated.

### Purification:

Purified by base-acid techniques.

TLC system: 10% MeOH: DCM R_{f} value: 0.5
Nature: Brown solid. Yield: 80 mg (32%)
Analytical Data: The product is confirmed by ¹HNMR and LCMS

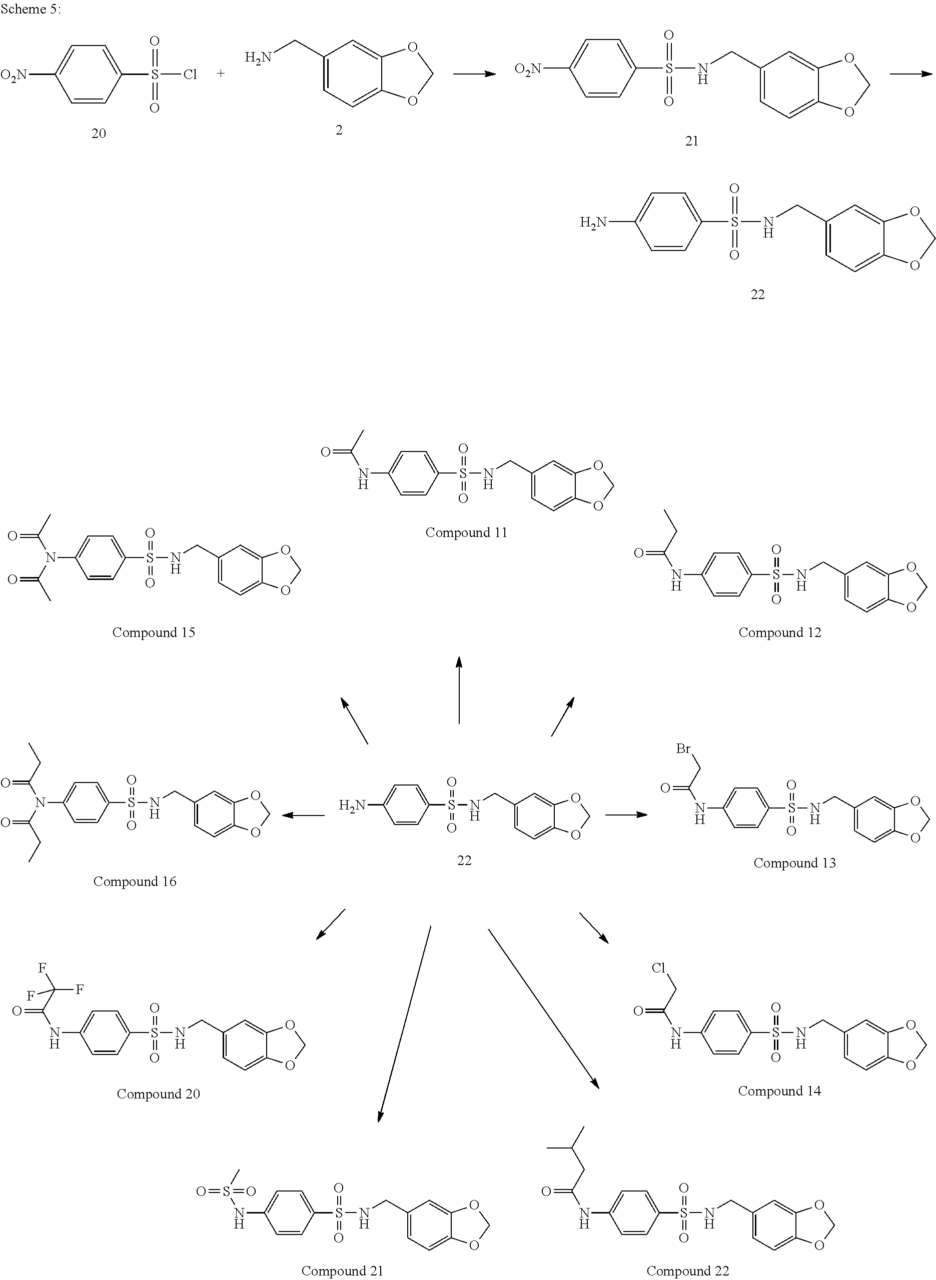

### Step 1

### Preparation of N-(benzo[d][1,3]dioxol-5-ylmethyl)-4-nitrobenzenesulfonamide (21)

| S.No | Chemicals/Reagents &Solvents | M.Wt | m.M | Eq. | Qty. | |
|---|---|---|---|---|---|---|
| 1. | Compound 2 | 151.65 | 6.59 | 1 | 1.0 | g |
| 2. | DCM | | | | 20 | ml |
| 3. | Triethyl amine | 101 | 9.89 | 1.5 | 0.99 | g |
| 4. | 4-Nitro benzene | 221.6 | 7.25 | 1.1 | 1.6 | g |
| | sulphonyl chloride | | | | | |

Reaction Time: 1 hour Reaction Temperature: 0° C. to rt
Brief procedure: In a clean dry RBF charged 1 in DCM under N₂ atm. cooled the flask to 0° C. and added 3. Stirred the reaction mixture for 5-10 min. Then 4 was added and maintained the reaction mixture for 1 hr. Reaction was monitored by TLC.
Work up: The reaction mixture was poured into ice water. Separated DCM layer, dried over anhydrous Na₂SO₄ and evaporated the solvent (DCM) under reduced pressure.
Purification: crude compound triturated with n-Hexane. Solid compound was filtered and was washed with n-hexane. Solid dried under reduced pressure.
TLC system: 40% EtOAc:Hexane R_{f} value: 0.3
Nature: Yellow solid. Yield: 2 g (90.9%)

### Step 2

### Preparation of 4-amino-N-(benzo[d][1,3]dioxol-5-ylmethyl)benzenesulfonamide (22)

| Sr.No | Chemicals/Reagents &Solvents | M.Wt | m.M | Eq. | Qty. | |
|---|---|---|---|---|---|---|
| 1. | Reactant 21 | 336.75 | 5.93 | 1 | 2 | g |
| 2. | EtOH:THF:H2O (4:2:1) | | | | 35 | mL |
| 3. | Iron powder (Fe) | 56 | 23.75 | 4 | 1.33 | g |
| 4. | Ammonium chloride | 53.5 | 20.75 | 3.5 | 1.11 | g |

### Reaction Time: 2 h Reaction Temperature: 80° C.

### Brief Procedure:

Reactant 21 was taken in EtOH:THF:H₂O (4:2:1) and then added Iron powder (Fe) and Ammonium chloride at RT (25° C.). The reaction mixture was brought to reflux for 2 hours. Reaction was monitored by TLC.

### Work Up:

The reaction mixture was concentrated to get crude compound. Crude compound was taken in EtOAc and was washed with water and brine solution. EtOAc layer dried over anhydrous Na₂SO₄ and the organic layer concentrated under reduced pressure.

Purification: crude compound purified by flash column chromatography using EtOAC/Hexane as mobile phase.
TLC system: 40% EtOAc: 60% Hexane R_{f} value: 0.2
Nature: Light brown solid. Yield: 1.2 g (65.93%)

### Step 3

### Preparation of N-(4-(N-(Benzo[d][1,3]dioxol-5-ylmethyl)sulfamoyl)phenyl)acetamide (Compound 11)

| S.No | Chemicals/Reagents &Solvents | M.Wt | m.M | Eq. | Qty. | |
|---|---|---|---|---|---|---|
| 1. | Reactant- 22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | Acetyl chloride | 78.5 | 0.31 | 1.2 | 24.5 | mg |

Reaction Time: 1 hour Reaction Temperature: 0° C. to rt.

### Brief Procedure:

Reactant 22(Int) was dissolved in DCM, TEA was added and stirred. The resulting solution was cooled to 0° C. and slowly drop wise was added acetyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.26
Nature: Light Brown solid. Yield: 31 mg (33.8%)

### Step 4

### Preparation of N-(4-(N-(Benzo[d][1,3]dioxol-5-ylmethyl)sulfamoyl)phenyl) propionamide (Compound 12)

| S.No | Chemicals/Reagents &Solvents | M.Wt | m.M | Eq. | Qty. | |
|---|---|---|---|---|---|---|
| 1. | Reactant-22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | Propionyl chloride | 92.5 | 0.31 | 1.2 | 28.7 | mg |

Reaction Time: 1 hour Reaction Temperature: 0° C. to rt.

### Brief Procedure:

Reactant 22(Int) was dissolved in DCM, TEA was added and stirred. The resulting solution was cooled to 0° C. and slowly drop wise was added propionyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.28
Nature: Yellow solid. Yield: 18 mg (19.14%)

### Step-5

### Preparation of N-(4-(N-(Benzo[d][1,3]dioxol-5-ylmethyl)sulfamoyl)phenyl) bromoacetamide (Compound 13)

| S.No | Chemicals/Reagents &Solvents | M.Wt | m.M | Eq. | Qty. | |
|---|---|---|---|---|---|---|
| 1. | Reactant-22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | Bromo acetyl bromide | 201.85 | 0.28 | 1.1 | 57.9 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added bromo acetyl bromide. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.3
Nature: Light Brown solid. Yield: 20 mg (18%)

### Step 6

### Preparation of Preparation of N-(4-(N-(Benzo[d][1,3]dioxol-5-ylmethyl)sulfamoyl) phenyl)chloroacetamide (Compound 14)

| S.No | Chemicals/Reagents &Solvents | M.Wt | m.M | Eq. | Qty. | |
|---|---|---|---|---|---|---|
| 1. | Reactant-22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | Chloro acetyl chloride | 112.9 | 0.286 | 1.1 | 32.2 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added chloroacetyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.3
Nature: Pale Yellow solid. Yield: 41 mg (41.4%)

### Step 7

### Preparation of N-acetyl-N-(4-(N-(Benzo[d][1,3]dioxol-5-ylmethyl)sulfamoyl)phenyl) acetamide

### Compound 15

| S.No | Chemicals/Reagents &Solvents | M.Wt | m.M | Eq. | Qty. | |
|---|---|---|---|---|---|---|
| 1. | Reactant- 22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | Acetyl chloride | 78.5 | 1.04 | 4.0 | 81 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added Acetyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.26
Nature: Light Brown solid. Yield: 16.7 mg (16.5%)

### Step 8

### Preparation of N-(4-(N-(Benzo[d][1,3]dioxol-5-ylmethyl)sulfamoyl)phenyl)-N-propionylpropionamide (Compound 16)

| S.No | Chemicals/Reagents &Solvents | M.Wt | m.M | Eq. | Qty. | |
|---|---|---|---|---|---|---|
| 1. | Reactant- 22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | Propionyl chloride | 92.5 | 1.04 | 4.0 | 96.5 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added propionyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.28
Nature: Light Green solid. Yield: 46 mg (42.2%)

### Step 9

### Preparation of N-(4-(N-(Benzo[d][1,3]-dioxol-5-ylmethyl)sulfamoyl)phenyl)-2,2,2-trifluoroacetamide (Compound 20)

| S.No | Chemicals/Reagents &Solvents | M.Wt | m.M | Eq. | Qty. | |
|---|---|---|---|---|---|---|
| 1. | Reactant- 22(Int) | 306.35 | 0.16 | 1 | 50 | mg |
| 2. | DCM | | | | 5 | mL |
| 3. | TEA | 101.1 | 0.21 | 1.3 | 21.4 | mg |
| 4. | Trifluoro acetic anhydride | 210.03 | 0.17 | 1.1 | 36.9 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added trifluoro acetic anhydride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.26
Nature: Off-White solid. Yield: 15 mg (21.9%)

### Step 10

### Preparation of N-(benzo[d][1,3]dioxol-5-ylmethyl)-4-(methylsulfonamido)benzenesulfonamide (Compound 21)

| S.No | Chemicals/Reagents &Solvents | M.Wt | m.M | Eq. | Qty. | |
|---|---|---|---|---|---|---|
| 1. | Reactant- 22(Int) | 306.35 | 0.26 | 1 | 80 | mg |
| 2. | THE | | | | 5 | mL |
| 3. | TEA | 101.1 | 0.33 | 1.3 | 34 | mg |
| 4. | Mesyl chloride | 114.5 | 0.28 | 1.1 | 32.7 | mg |

### Reaction Time: 1 h Reaction Temperature: −78° C.

### Brief Procedure:

Reactant 22(Int) was dissolved in DCM, TEA was added and stirred. The resulting solution was cooled to 0° C. and slowly drop wise was added Mesyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAC: 60% Hexane R_{f} value: 0.24
Nature: Light Brown solid. Yield: 28 mg (28%)

### Step 11

### Preparation of N-(4-(N-(Benzo[d][1,3]dioxol-5-ylmethyl)sulfamoyl)phenyl)-3,3-dimethylbutanamide (Compound 22)

| S.No | Chemicals/Reagents &Solvents | M.Wt | m.M | Eq. | Qty. | |
|---|---|---|---|---|---|---|
| 1. | Reactant- 22(Int) | 306.35 | 0.52 | 1 | 160 | mg |
| 2. | DCM | | | | 10 | mL |
| 3. | TEA | 101.1 | 0.33 | 1.3 | 68 | mg |
| 4. | 3,3-Dimethyl butanoyl chloride | 134.6 | 0.57 | 1.1 | 76.9 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added 3,3-dimethyl butanoyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAC: Hexane R_{f} value: 0.26
Nature: Pale Yellow solid. Yield: 76 mg (36%)

### Step 12

### Preparation of N-(4-(N-(Benzo[d][1,3]dioxol-5-ylmethyl)sulfamoyl)phenyl)-3-methyl butanamide (Compound 22A)

| S.No | Chemicals/Reagents &Solvents | M.Wt | m.M | Eq. | Qty. | |
|---|---|---|---|---|---|---|
| 1. | Reactant- 22(Int) | 306.35 | 0.26 | 1 | 80 | mg |
| 2. | DCM | | | | 5 | mL |
| 3. | TEA | 101.1 | 0.33 | 1.3 | 34 | mg |
| 4. | 3-methyl butanoyl chloride | 120.57 | 0.28 | 1.1 | 34.48 | mg |

### Reaction Time: 1 h Reaction Temperature: 0° C. to rt.

### Brief Procedure:

Reactant 22(Int) was dissolved in DCM, TEA was added and stirred. The resulting solution was cooled to 0° C. and slowly drop wise was added 3-methyl butanoyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAC: Hexane R_{f} value: 0.3
Nature: Off-White solid. Yield: 13.8 mg (13.5%)

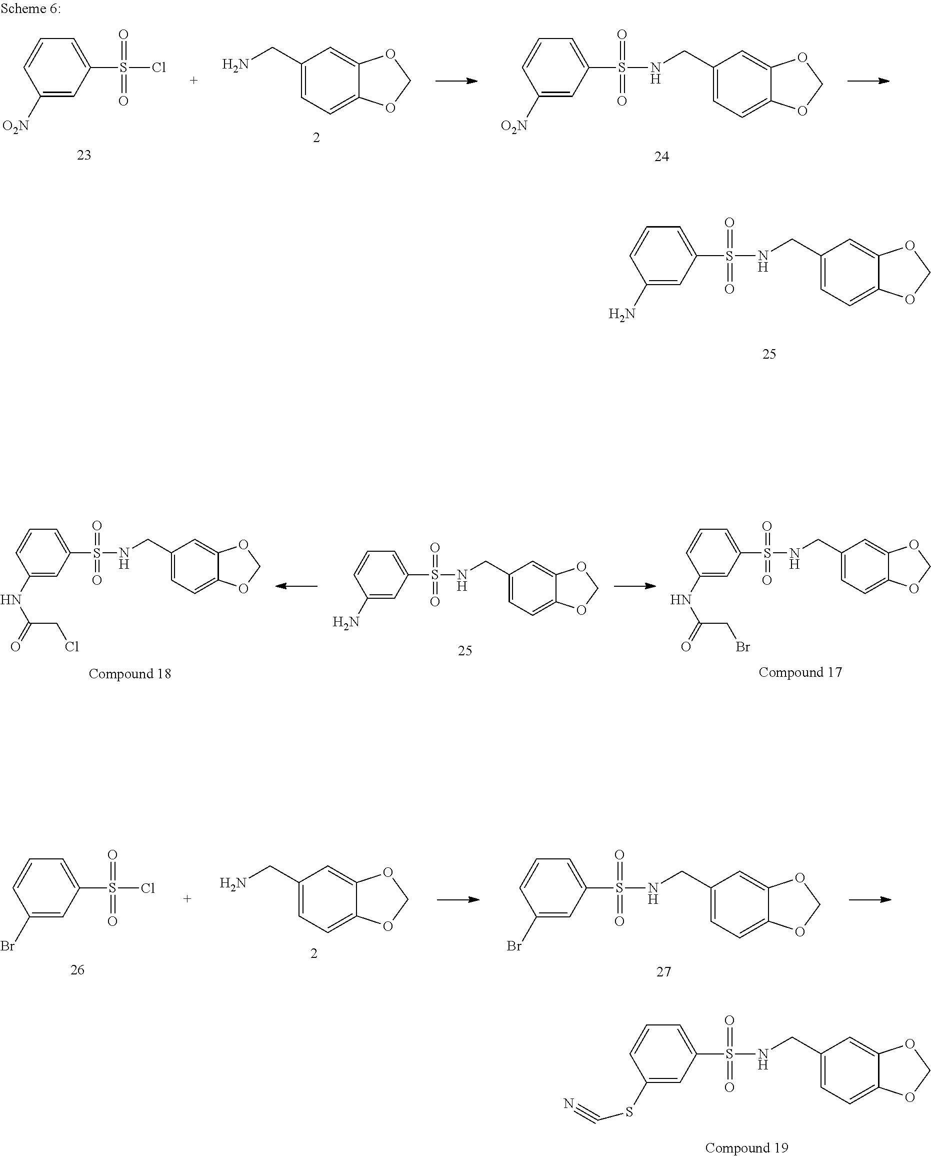

### Step 1

### Preparation of N-(benzo[d][1,3]dioxol-5-ylmethyl)-3-nitrobenzenesulfonamide (24)

| S.No | Chemicals/Reagents &Solvents | M.Wt | m.M | Eq. | Qty. | |
|---|---|---|---|---|---|---|
| 1. | Reactant- 2 | 151.65 | 3.29 | 1 | 0.5 | g |
| 2. | DCM | | | | 15 | mL |
| 3. | Triethyl amine | 101 | 4.9 | 1.5 | 0.49 | g |
| 4. | 3-Nitro benzene | 221.6 | 3.6 | 1.1 | 0.8 | g |
| | sulphonyl chloride | | | | | |

### Reaction Time: 1 Reaction Temperature: 0° C. to rt.

Brief procedure: In a clean dry RBF was charged with compound 2 in DCM under N2 atm. Cooled the flask to 0° C. and was added triethyl amine. Reaction mixture was stirred for 5-10 min. Then 3-Nitro benzene sulphonyl chloride was added and maintained the reaction mixture for 1 hr. Reaction was monitored by TLC.
Work up: The reaction mixture was poured into ice water. Separated DCM layer, dried over anhydrous Na₂SO₄ and evaporated the solvent (DCM) under reduced pressure.
Purification: crude compound triturated with n-Hexane. Solid compound was filtered and was washed with n-hexane. Solid dried under reduced pressure.
TLC system: 40% EtOAc:Hexane R_{f} value: 0.3
Nature: Yellow solid. Yield: 1 g (90.9%)

### Step 2

### Preparation of 3-amino-N-(benzo[d][1,3]dioxol-5-ylmethyl)benzenesulfonamide (25)

| | Chemicals/Reagents | | | | |
|---|---|---|---|---|---|
| S. No | & Solvents | M. Wt | m.M | Eq. | Qty. |
| 1. | Reactant-24 | 336.75 | 2.965 | 1 | 1 g |
| 2. | EtOH:THF:H2O (4:2:1) | | | | 20 mL |
| 3. | Iron powder (Fe) | 56 | 11.875 | 4 | 0.665 g |
| 4. | Ammonium chloride | 53.5 | 10.375 | 3.5 | 0.556 g |

### Reaction Time: 2 h Reaction Temperature: 80° C.

### Brief Procedure:

Compound 24 was taken in EtOH:THF:H₂O (4:2:1) and then was added Iron powder (Fe) and ammonium chloride at RT (25° C.). Heated the mixture to reflux for 2 hours. Reaction was monitored by TLC.

### Work Up:

The reaction mixture was concentrated to get crude compound. Crude compound was taken in EtOAc and washed with water and brine solution. EtOAc layer dried over anhydrous Na₂SO₄ and the organic layer was concentrated under reduced pressure.

Purification: crude compound was purified by flash column chromatography using EtOAc/Hexane as mobile phase.
TLC system: 40% EtOAc:Hexane R_{f} value: 0.2
Nature: Light brown solid. Yield: 0.6 g (65.93%)

### Step 3

### Preparation of N-(3-(N-(Benzo[d][1,3]dioxol-5-ylmethyl)sulfamoyl)phenyl)-2-bromoacetamide (Compound 17)

| | Chemicals/Reagents | | | | |
|---|---|---|---|---|---|
| S. No | & Solvents | M. Wt | m.M | Eq. | Qty. |
| 1. | Reactant-25(Int) | 306.35 | 0.326 | 1 | 100 mg |
| 2. | DCM | | | | 10 mL |
| 3. | TEA | 101.1 | 0.423 | 1.3 | 42.8 mg |
| 4. | Bromo acetyl bromide | 201.85 | 0.358 | 1.1 | 72.3 mg |

### Reaction Time: 1 h Reaction Temperature: 0° C. to rt.

### Brief Procedure:

Reactant 25(Int) was dissolved in DCM, TEA was added and stirred. The resulting solution was cooled to 0° C. and slowly drop wise was added bromoacetyl bromide. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.26
Nature: white solid. Yield: 41 mg (29.4%)

### Step 4

### Preparation of N-(3-(N-(Benzo[d][1,3]dioxol-5-ylmethyl)sulfamoyl)phenyl)-2-chloro acetamide (Compound 18)

| | Chemicals/Reagents | | | | |
|---|---|---|---|---|---|
| S. No | & Solvents | M. Wt | m.M | Eq. | Qty. |
| 1. | Reactant-25(Int) | 306.35 | 0.326 | 1 | 100 mg |
| 2. | DCM | | | | 10 mL |
| 3. | TEA | 101.1 | 0.423 | 1.3 | 42.9 mg |
| 4. | Chlroacetyl chloride | 112.9 | 0.358 | 1.1 | 40.4 mg |

### Reaction Time: 1 h Reaction Temperature: 0° C. to rt.

### Brief Procedure:

Reactant 25(Int) was dissolved in DCM, TEA was added and stirred. The resulting solution was cooled to 0° C. and slowly drop wise was added chloroacetyl chloride. The reaction mixture was allowed to warm to room temperature and was stirred for 1 hour. Reaction was monitored by TLC.

### Work Up:

The reaction mixture was poured into water and extracted with DCM. The combined organic layers were dried over Na₂SO₄. The solvent was concentrated under reduced pressure.

Purification: Purified by combiflash using hexane and ethyl acetate as mobile phase.
TLC system: 40% EtOAc:Hexane R_{f} value: 0.28
Nature: white solid. Yield: 46 mg (36.8%)

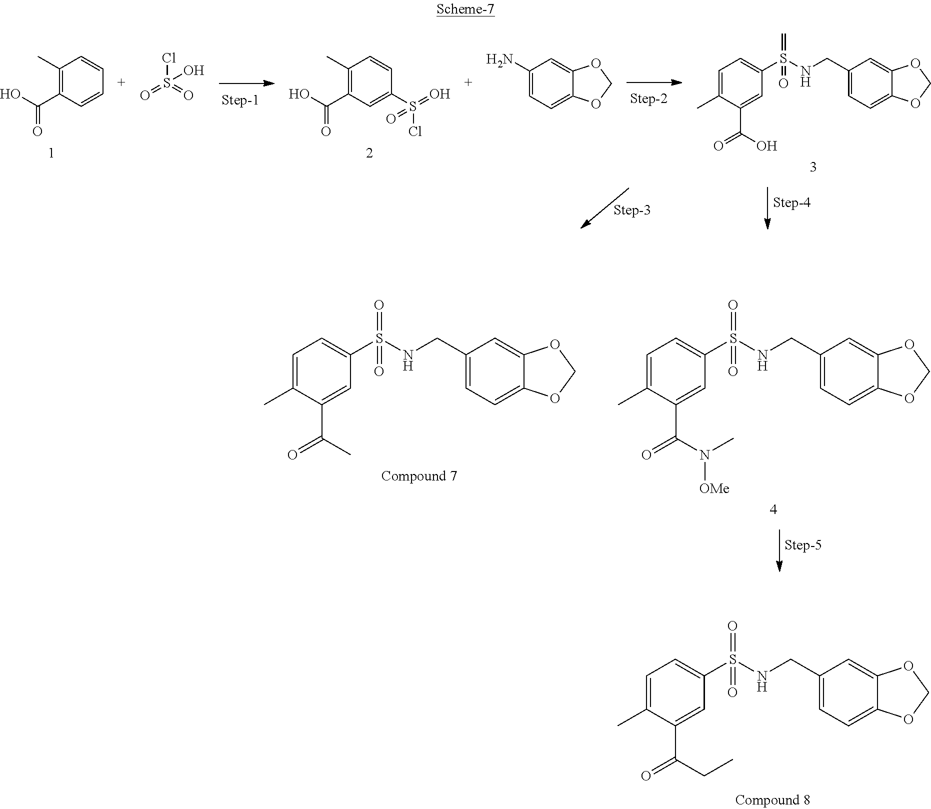

### Step 1: 5-(chlorosulfonyl)-2-methylbenzoic acid (2)

| Sr. No | Chemicals/Reagents & Solvents | M. Wt | m.M | Eq. | Qty. |
|---|---|---|---|---|---|
| 1. | O-Toluic acid | 136 | 22.0 | 1 | 3 gm |
| 2. | Chlorosulfonicacid | 116 | 44.0 | 2 | 4.5 mL |

### Reaction Time: 1 hrs Reaction Temperature: rt to 100° C.

Brief procedure: O-Toluic acid was dissolved in chlorosulfonic acid at rt. Then the reaction mixture was heated to 100° c. The reaction mixture was stirred for 1 hr.
Work up: The reaction mixture was quenched with ice cold water. The compound was precipitated as white solid. The solid compound was filtered and washed with n-Hexane.
Purification: used for next step without any further purification
TLC system: 10% MeOH in DCM R_{f} value: 0.3
Nature: white solid. Yield: 3 gm (60%)

### Step 2: 5-(N-(benzo[d][1,3]dioxol-5-ylmethyl)sulfamoyl)-2-methylbenzoic acid (3)

| Sr. No | Chemicals/Reagents & Solvents | M. Wt | m.M | Eq. | Qty. |
|---|---|---|---|---|---|
| 1. | Compound2 | 234 | 14.56 | 1 | 3 gm |
| 2. | Amine | 151 | 13.24 | 1.2 | 2 gm |
| 3. | TEA | 101 | 26.48 | 2 | 3.1 mL |
| 4. | DCM | | | | 60 mL |

### Reaction Time: 1 h Reaction Temperature: 0° C. to rt.

Brief procedure: Compound 2 was dissolved in DCM and TEA was added. The resulting solution was then cooled approximately to 0° c. and treated drop wise with a solution of amine. The reaction mixture was allowed to warm to room temperature and stirred for 12 hour.

### Work Up:

The reaction mixture was then poured into water and extracted with DCM. The combined organic layers were washed with water followed by brine and dried over MgSO₄. The solvent was removed in vacuum.

Purification: Purified through flash column chromatography using hexane and ethyl acetate as mobile phase.
TLC system: 50% EtOAc:Hexane R_{f} value: 0.3
Nature: White solid. Yield: 500 mg (20%)

### Step 3: 3-acetyl-N-(benzo[d][1,3]dioxol-5-ylmethyl)-4-methylbenzenesulfonamide (compound 7)

| | Chemicals/Reagents | | | | |
|---|---|---|---|---|---|
| Sr. No | & Solvents | M. Wt | m.M | Eq. | Qty. |
| 1. | Int-3 | 347 | 1.152 | 1 | 400 mg |
| 2. | Methyl lithium | 23 | 3.48 | 3 | 2.1 mL |
| 3. | THF | | | | 10 mL |

Reaction Time: 12 hour Reaction Temperature: 0° C. to rt.
Brief procedure: Int-3 was dissolved in THF, Methyllithium was added slowly to the above reaction mixture at rt. The reaction mixture was stirred for 1 hr at rt.
Work up: The reaction mixture was then poured into water and extracted with EtOAc. The combined organic layers washed with water followed by brine and dried over MgSO₄. The solvent is removed in vacuum to give the titled crude product
Purification: Purified through flash column chromatography using hexane and ethyl acetate as mobile phase.
TLC system: 30% EtOAc:Hexane R_{f} value: 0.6
Nature: White solid. Yield: 50 mg (15%)

### Step 4: Preparation of 5-(N-(benzo[d][1,3]dioxol-5-ylmethyl)sulfamoyl)-N-methoxy-N,2-dimethylbenzamide (4)

| | Chemicals/Reagents | | | | |
|---|---|---|---|---|---|
| Sr. No | & Solvents | M. Wt | m.M | Eq. | Qty. |
| 1. | Int-3 | 347 | 2.88 | 1 | 1 gm |
| 2. | CDI | 162 | 5.76 | 2 | 0.92 gm |
| 3. | N,O- dimethyloxylamine | 97.5 | 5.76 | 2 | 0.6 gm |
| | hydrochloride | | | | |
| 4. | Acetonitrile | | | | 20 mL |

Brief procedure: Int-3 was dissolved in acetonitrile. CDI was added portion wise at 0° C. The resulting solution was stirred for 1 hr at rt. Then the reaction mass was cooled to 0° C. and N,O-dimethyloxylamine hydrochloride was added portion wise. The reaction mixture was stirred for 4 hrs at RT.

### Work Up:

The reaction mixture was then poured into water and extracted with EtOAc. The combined organic layers were washed with water followed by brine and dried over MgSO₄. The solvent was removed in vacuum.

Purification: Purified through flash column chromatography. using hexane and ethyl acetate as mobile phase.
TLC system: 50% EtOAc:Hexane R_{f} value: 0.4
Nature: White solid. Yield: 500 mg (45%)

### Step 4: Preparation of N-(benzo[d][1,3]dioxol-5-ylmethyl)-4-methyl-3-propionylbenzenesulfonamide (compound 8)

| Sr. No | Chemicals/Reagents & Solvents | M. Wt | m.M | Eq. | Qty. |
|---|---|---|---|---|---|
| 1. | Int-4 | 390 | 1.282 | 1 | 500 mg |
| 2. | ethylmagnisiumbromide(1M) | 135 | 2.564 | 2 | 2 mL |
| 3. | THF | | | | 10 mL |

Reaction Time: 1 hour. Reaction Temperature: 0° C.

### Brief Procedure:

The Int-4 was dissolved in THE and ethylmagnisiumbromide (1M) was added at 0° C. The reaction mixture was stirred for 16 hr at rt

### Work Up:

The reaction mixture was then poured into water and extracted with EtOAc. The combined organic layers were washed with water followed by brine and dried over MgSO₄. The solvent was removed in vacuum.

Purification: Purified through flash column chromatography using hexane and ethyl acetate as mobile phase.
TLC system: 50% EtOAc:Hexane R_{f} value: 0.5
Nature: White solid. Yield: 50 mg

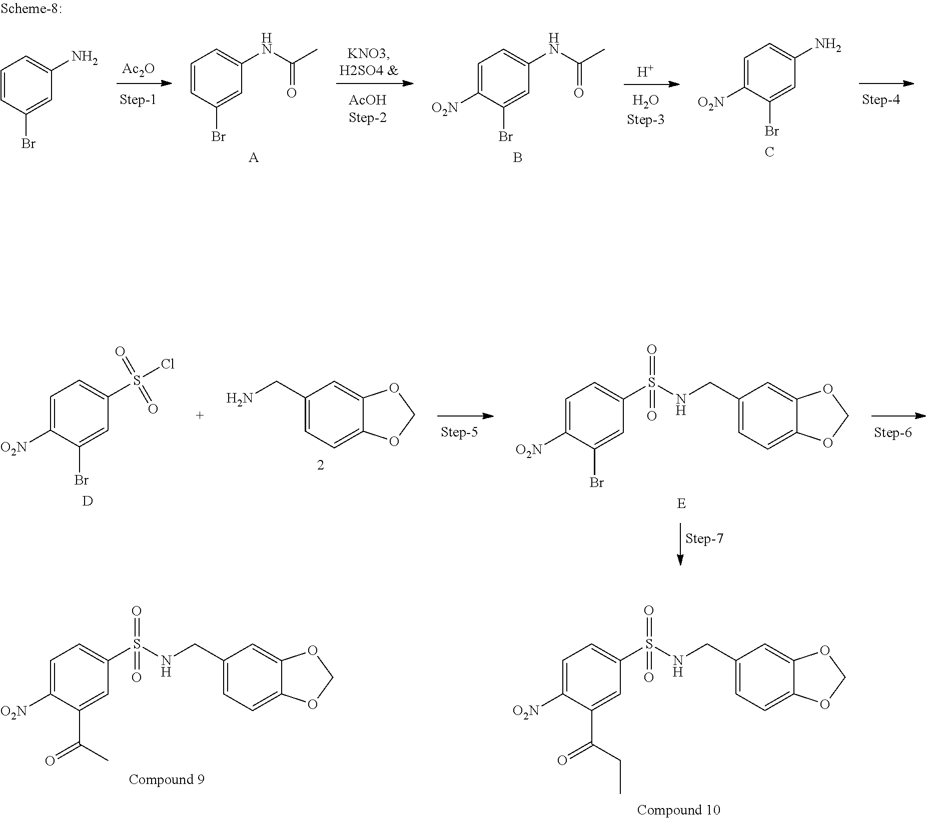

### Step 1: N-(3-bromophenyl)acetamide (A)

| S. No | Chemicals/Reagents & Solvents | M. Wt | m.M | Eq. | Qty. |
|---|---|---|---|---|---|
| 1. | 3-Bromo Aniline | 172.03 | 58.1 | 1.0 | 10.0 g |
| 2. | Acetic solvent | 102 | 116.2 | 2.0 | 11.0 mL |
| 3. | H2SO4 | | | | 1.0 mL |

Reaction Time: 15 mins Reaction Temperature: 0-5° C.
Brief procedure: A mixture of 3-Bromo Aniline, acetic solvent and H₂SO₄ was stirred for 15 mins. Reaction was monitored by TLC.
Work up: The reaction mixture was poured on to ice, the compound was precipitated. Then the compound was filtered, washed with water (50 ml) & dried.
TLC system: 40% EtOAc:Hexane R_{f} value: 0.4
Nature: Off-white solid Yield: 9.5 g (76%)

### Step 2: N-(3-bromo-4-nitrophenyl)acetamide (B)

| S. No | Chemicals/Reagents & Solvents | M. Wt | m.M | Eq. | Qty. |
|---|---|---|---|---|---|
| 1. | N-(3-Bromophenyl)acetamide A | 214 | 37.38 | | 8.0 g |
| 2. | H₂SO₄ | | | | 40.0 mL |
| 3. | KNO₃ | 101 | 41.1 | 1.1 | 4.1 g |
| 4. | Glacial AcOH | | | | 15.0 mL |

Reaction Time: 30-60 mins Reaction Temperature: 0-5° C.
Brief procedure: A mixture of N-(3-Bromophenyl)Acetamide A, glacial AcOH & 10.0 mL of H₂SO₄ were cooled to 0-5° C., added cold KNO3 in H₂SO₄ (30 mL). Reaction was monitored by TLC.
Work up: The reaction mixture was poured on to ice, the compound was precipitated. Then the compound was filtered, washed with water (50 mL) & dried.
Purification: The crude was purified using silica gel flash column chromatography. Our desired compound was eluted with 40% EtOAc:Hexane
TLC system: 40% EtOAc:Hexane R_{f} value: 0.2
Nature: Yellow solid Yield: 2.0 g (20%)

### Step 3: 3-bromo-4-nitroaniline (C)

| S. No | Chemicals/Reagents & Solvents | M. Wt | m.M | Eq. | Qty. |
|---|---|---|---|---|---|
| 1. | N-(3-Bromo-4- | 259 | 3.8 | | 1.0 g |
| | nitrophenyl)acetamide B | | | | |
| 2. | H₂SO₄ | | | | 10.0 mL |
| 3. | H₂O | | | | 30.0 mL |

### Reaction Time: 2 hrs Reaction Temperature: 90-100° C.

Brief procedure: A mixture of N-(3-Bromo-4-nitrophenyl)acetamide C, H₂SO₄ & H2O was refluxed at 90° C. for 2 hrs.
Work up: The reaction mixture was poured onto ice bath, the compound was precipitated, filtered, purified using silica gel flash column chromatography.
TLC system: 50% EtOAc:Hexane R_{f} value: 0.5
Nature: Yellow solid Yield: 350 mg (50%)

### Step 4: 3-bromo-4-nitrobenzene-1-sulfonyl chloride (D)

| | Chemicals/Reagents & | | | | |
|---|---|---|---|---|---|
| S. No | Solvents | M. Wt | m.M | Eq. | Qty. |
| 1. | 3-Bromo-4-nitro Aniline C | 217 | 1.1 | 1.0 | 0.25 g |
| 2. | NaNO2 | 68.9 | 1.1 | 1.0 | 0.8 g in |
| | | | | | 1 ml H2O |
| 3. | Glacial AcOH | | | | 8.0 ml |
| 4. | HCl | | | | 3.0 ml |
| 5. | SO2/AcOH/CuC12/H2O | | | | 20.0 ml |

Reaction Time: 30-60 mins Reaction Temperature: 0-5° C.
Brief procedure: HCl was added to the mixture of 3-Bromo-4-nitro Aniline C & glacial AcOH at 0° C., added NaNO2 in H2O dropwise, stirred for 15 mins & then SO2 solution was added dropwise.
Work up: Cold H2O (20 mL) was added, extracted with Et2O (20 mL×3). The combined organic layers were washed with cold H2O (20 mL×3), dried over Na2SO4 & evaporated
TLC system: 30% EtOAc:Hexane R_{f} value: 0.6
Nature: Yellow solid Yield: 140 g (20%)

### Step 5: N-(benzo[d][1,3]dioxol-5-ylmethyl)-3-bromo-4-nitrobenzenesulfonamide (E)

| S. No | Chemicals/Reagents & Solvents | M. Wt | m.M | Eq. | Qty. |
|---|---|---|---|---|---|
| 1. | 3-Bromo-4-nitrobenzene-1-sulfonyl | 300 | 0.4 | 1.0 | 140 mg |
| | chloride D | | | | |
| 2. | 2 | 151.6 | 0.5 | 1.1 | 0.06 mL |
| 3. | Triethyl amine | 101.1 | 0.6 | 1.3 | 0.06 mL |
| 4. | DCM | | | | 10.0 mL |

Reaction Time: 30-60 mins Reaction Temperature: 0-5° C.
Brief procedure: Compound 2 was dissolved in DCM and TEA was added and stirred. The resulting solution was then cooled approximately to 0° C. and treated drop wise with a solution of D. The reaction mixture is allowed to warm to room temperature and is stirred for 12 hour.
Work up: The reaction mixture was then poured into water and extracted with DCM. The combined organic layers were washed with water followed by brine and dried over MgSO₄. The solvent was removed in vacuum.
Purification: Purified through flash column chromatography using hexane and ethyl acetate as mobile phase.
TLC system: 50% EtOAc:Hexane R_{f} value: 0.6
Nature: Yellow solid Yield: 140 g (73%)

### Step 6: 3-acetyl-N-(benzo[d][1,3]dioxol-5-ylmethyl)-4-nitrobenzenesulfonamide (compound9)

| | Chemicals/Reagents | | | | |
|---|---|---|---|---|---|
| S. No | & Solvents | M. Wt | m.M | Eq. | Qty. |
| 1. | N-(benzo[d]dioxol-5- | 415.22 | 0.169 | 1 | 70 mg |
| | ylmethyl)-3-bromo-4- | | | | |
| | nitrobenzenesulfonamide E | | | | |
| 2. | tributyl(1-ethoxyvinyl) tin | 361.4 | 0.338 | 2 | 0.12g |
| 3. | Tetrakistriphenyl- | 1155.58 | 0.0084 | 0.05 | 0.009 |
| | phosphinepalladium | | | | |
| 4. | 1,4-dioxane | | | | 80 mL |

Reaction Time: 16 hour. Reaction Temperature: 85° C.

### Brief Procedure:

Int-E, tributyl (1-ehoxyvinyl) tin and tetrakistriphenylphosphinepalladium were added in dioxin in a seal tube. The reaction mixture was stirred overnight at 85° C., then diluted with ethyl acetate, washed with sat. NaHCO₃ and brine, dried over Na₂SO₄ and evaporated. The residue was dissolved in THF (10 mL), 1N HCl was added and the mixture was stirred for 18 h. The solvent was removed in vacuum.

Purification: Purified through flash column chromatography using hexane and ethyl acetate as mobile phase.
TLC system: 50% EtOAc:Hexane R_{f} value: 0.3
Nature: Pale yellow solid. Yield: 15 mg

### Step 7: N-(benzo[d][1,3]dioxol-5-ylmethyl)-4-nitro-3-propionylbenzenesulfonamide (compound10)

| | Chemicals/Reagents | | | | |
|---|---|---|---|---|---|
| S. No | & Solvents | M. Wt | m.M | Eq. | Qty. |
| 1. | N-(benzo[d]dioxol-5- | 415.22 | 0.169 | 1 | 70 mg |
| | ylmethyl)-3-bromo-4- | | | | |
| | nitrobenzenesulfonamide E | | | | |
| 2. | tributyl(1-ethoxyprop-1- | 376.4 | 0.338 | 2 | 0.12 |
| | enyl) tin | | | | |
| 3. | Tetrakistriphenyl- | 1155.58 | 0.0084 | 0.05 | 0.009 |
| | phosphinepalladium | | | | |
| 4. | 1,4-dioxane | | | | 80 mL |

Reaction Time: 16 hour. Reaction Temperature: 85° C.

### Brief Procedure:

Int-E, tributyl(1-ethoxyprop-1-enyl) tin and tetrakistriphenylphosphinepalladium were added in dioxin in a seal tube. The reaction mixture was stirred overnight at 85° C., then diluted with ethyl acetate, washed with sat. NaHCO₃ and brine, dried over Na₂SO₄ and evaporated. The residue was dissolved in THF (10 mL), 1N HCl was added and the mixture was stirred for 18 h. The solvent was removed in vacuum.

Purification: Purified through flash column chromatography using hexane and ethyl acetate as mobile phase.
TLC system: 50% EtOAc:Hexane R_{f} value: 0.3
Nature: Pale yellow solid Yield: 19 mg
The compounds of the invention can also be synthesized by methods following the synthetic schemes below:

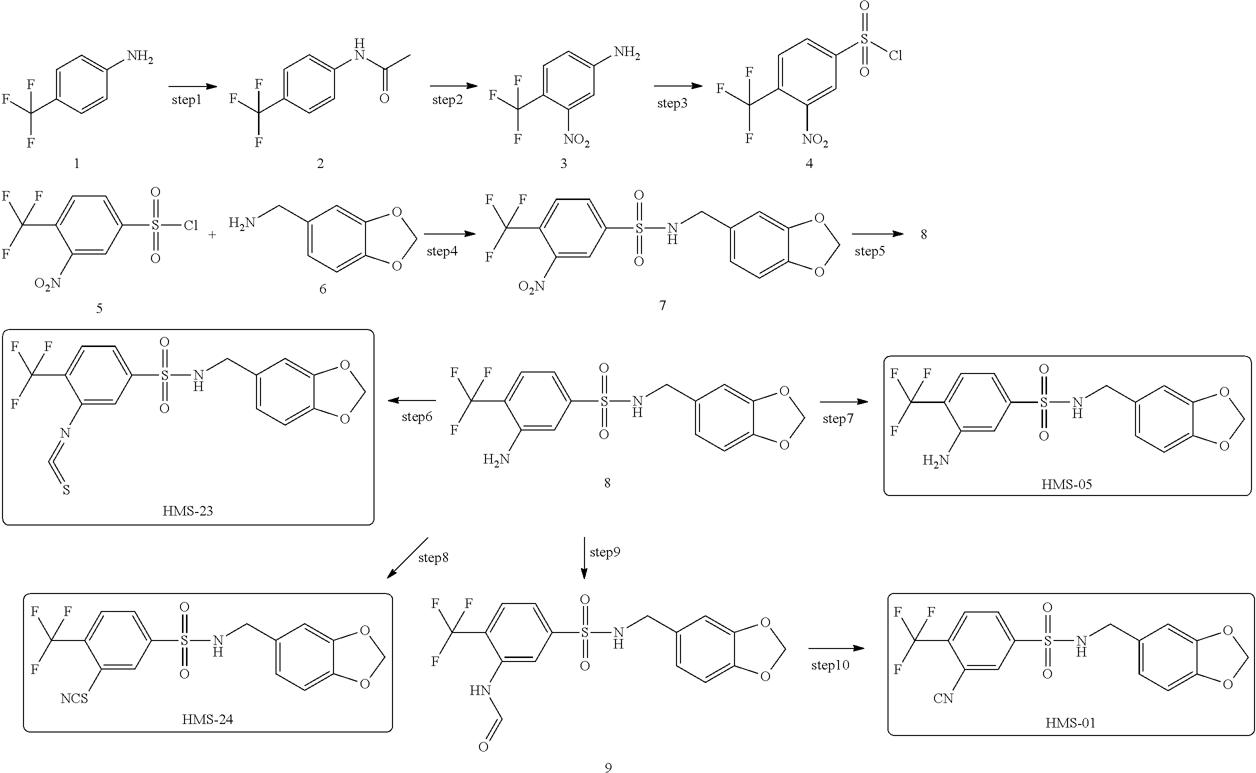

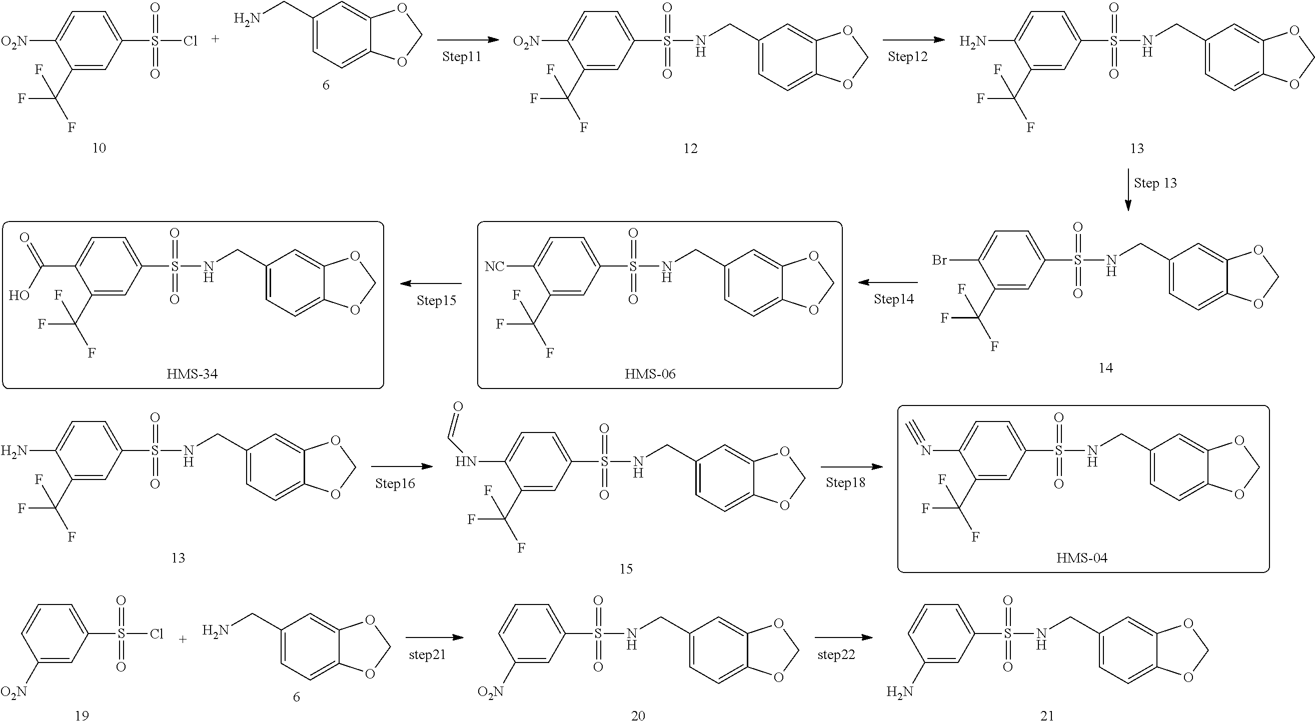

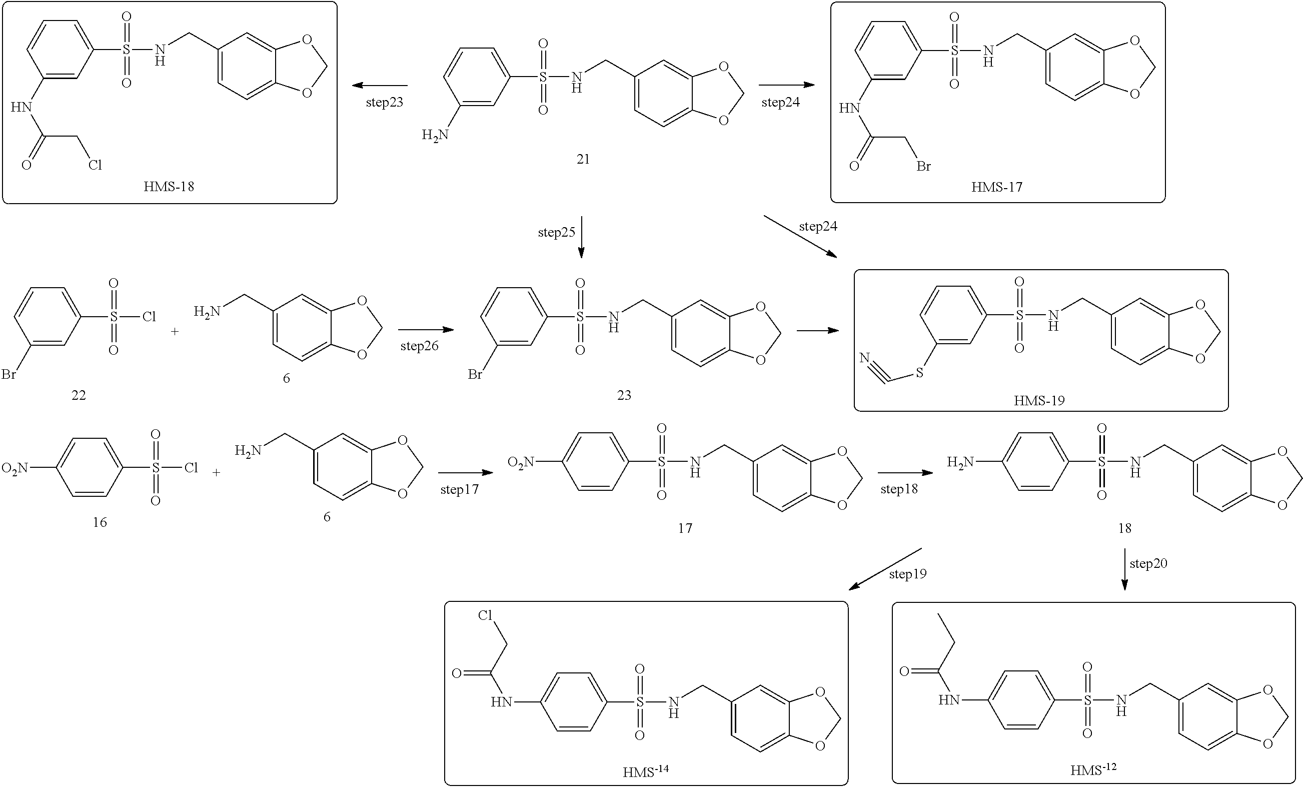

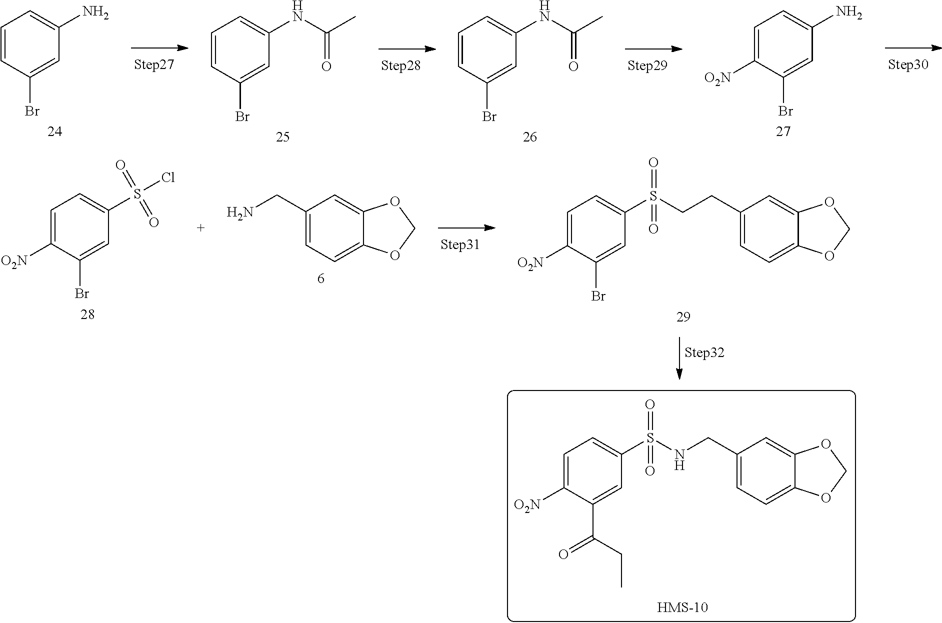

### Example 2

### Biological Data

### AR Homology Modeling

The refined homolog model of the AR in an antagonized conformation was constructed using the comparative protein modeling suite ORCHESTRAR (SYBYL 8.0, Tripos International, 1699 South Hanley Rd., St. Louis, Mo., 63144, USA.). The crystal structure of agonist bound AR (PDB code: 2AM9) as well as the crystal structure of progesterone receptor in complex with the corepressor NCoR (PDB code: 2OVM) were used as template structures in generating and validating the model (Kauppi, B., et al. J Biol Chem 278, 22748-22754 (2003); Madauss, K. P., et al. Mol Endocrinol 21, 1066-1081 (2007)). After searching the loops and fixing the side chains, the obtained structure was minimized with MMFF94 forcefield using the Biopolymer module (Halgren, T. A. J. Comput. Chem. 17, 490-519 (1996).). The AR model was submitted to PROCHECK and the quality of the protein structure evaluated using a Ramachandran plot (Laskowski, R. A., MacArthur, M. W., Moss, D. S. & Thornton, J. M. Journal of Applied Crystallography 26, 283-291 (1993).). We determined that the refined homology model of the AR in the antagonized conformation had an RMSD of 3.627 Å when superimposed with the wildtype AR structure.

### Ligand-Based Pharmacophore Development

A training set of 23 active reference compounds were subjected to the pharmacophore alignment using the GALAHAD (Richmond, N., et al. Journal of Computer-Aided Molecular Design 20, 567-587 (2006).). Briefly, the molecules were aligned using the constellation of ligand pharmacophore descriptors, and single conformer pharmacophore models were generated using triplet tuplets. These triplet tuplet pharmacophores were generated using the point features including: positive nitrogens, negative centers, hydrogen bond donors, acceptors atoms; and hydrophobic centers with the multiple edge lengths binned at 0.5 Å intervals (Richmond, N. J., Willett, P. & Clark, R. D. Journal of Molecular Graphics and Modelling 23, 199-209 (2004).). One cluster of our virtual repository of 41300 compounds (˜400 k) were minimized using CONCORD (R. S. Pearlman, “Concord,” distributed by Tripos International, St. Louis, Mo., 63144, USA.) and subjected to the tuplet conformer generation (Abrahamian, E., et al. Journal of Chemical Information and Computer Sciences 43, 458-468 (2003).). These 3D pharmacophoric tuplet hypotheses were generated with the 100 random conformations of the active molecules resulting in a hit list of 4130 compounds (top 1% of chemical repository) that were then docked into our AR LBD homology model using SBVSs.

### Structure-Based Virtual Screening

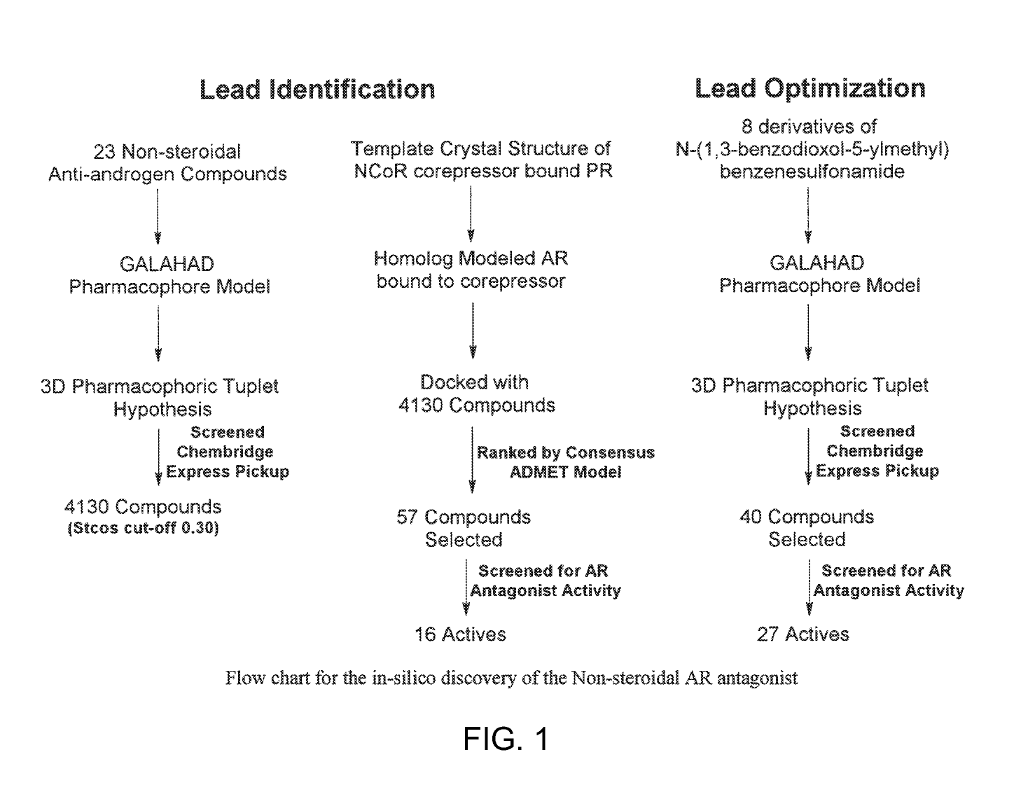

The hits from our pharmacophore screens were subjected to structure-based docking evaluations using our AR homology model and cross docked into the variant AR crystal structure bound to bicalutamide using the Surflex 2.4 suite (Jain, A. N. J Med Chem 46, 499-511 (2003).). Docking carried out with the initial generation of protomol with threshold range of 0.50 Å. Furthermore prior to docking, the ligands were energy minimized using the Tripos force field that employs Powell minimization and simplex optimization with a distance dependent dielectric function and an energy gradient of 0.01 kcal/molÅ with a maximum of 1000 iterations (Powell, M. J. D. Mathematical Programming 12, 241-254 (1977).). The top 4130 compounds from the primary LBVSs were further selected for use in subsequent in silico ADMET evaluation (SARCHITECT 2.5; Strand Biosciences, Bellary Road, Hebbal, Bangalore 560024, INDIA). Final compound ranking was based consensus ranking on our normalized total interaction energy from docking as well as, ADMET ranking that used drug-like truPk and truTox properties (Subramanian, K. Expert Opinion on Drug Metabolism & Toxicology 1, 555-564 (2005). The schematic work flow for the in-silico discovery of Non-steroidal AR antagonist is illustrated in FIG. 1.

### Cell Culture

We cultured COS7 cells (American Type Culture Collection CRL-1651) in DMEM media containing 10% FBS. We cultured PC3-AR, C4-2, and VCaP in RPMI media containing 10% FBS.

### Plasmid Constructs

The AR-responsive firefly luciferase reporter consists of 4 tandem repeats of the consensus steroid response element cloned upstream of the pGL3-Basic Luciferase vector (Promega). The AR WT construct expresses full-length human AR under cmv promoter control. AR N/C interaction assay constructs and the VP16-AR plasmid were generated using vectors from the Mammalian Matchmaker Two-Hybrid Assay Kit (Clontech 630301). The T887A, W741C, and AR□H12 mutant constructs were generated using the QuikChange II Site-Directed Mutagenesis system (Stratagene 200524).

### Transfection and Reporter Assays

We transfected cells using Lipofectamine 2000 (Invitrogen 11668). Luciferase activity was determined using the Dual-Luciferase Reporter Assay System (Promega E1910).

### Nuclear/Cytoplasmic Fractionation Assays

We used the NE-PER Nuclear and Cytoplasmic Extraction Reagents (Pierce 78833).

(II)

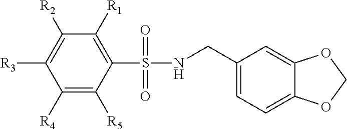

| | | | | | | | % Angtag |
|---|---|---|---|---|---|---|---|
| | | | | | | | (12.5 μM |
| En- | | | | | | logP | vs. 1 nM |
| try | R₁ | R₂ | R₃ | R₄ | R₅ | (o/w) | DHT) |
| 1 | | | Cl | | | 3.0860 | |
| 2 | | | F | | | 2.6470 | |
| 3 | | CH₃ | | | CH₃ | 3.1250 | |
| 4 | | | Br | | | 3.2920 | |
| 5 | | Cl | | | | 3.1230 | |
| 6 | | Cl | F | | | 3.2740 | |
| 7 | | CH₃ | | | | 2.8290 | |
| 8 | | CH₃ | F | | | 2.9800 | |
| 9 | | | CH₃ | | | 2.7920 | |
| 10 | | CH₃ | Cl | CH₃ | | | |
| 11 | | CH₃ | CH₃ | | | 3.1250 | 60 |
| 12 | Cl | Cl | | | | | |
| 13 | CH₃ | | Br | | | 3.6250 | 70 |
| 14 | Cl | | Cl | | | 3.7130 | 90 |
| 15 | Br | | Br | | | 4.1250 | 85 |
| 16 | OCH₃ | Br | | | | | |
| 17 | | | OCH₃ | | | 2.4500 | 20 |
| 18 | Cl | CH₃ | Cl | | | | |
| 19 | | | I | | | 3.6840 | 70 |
| 20 | OCH₃ | Cl | Cl | | | 3.6280 | 0 |
| 21 | Cl | | Cl | Cl | | 4.3400 | 70 |
| 22 | | OCH₃ | Cl | | | 3.0770 | 70 |
| 23 | Cl | Cl | Cl | | | 4.2640 | 75 |
| 24 | Cl | Cl | OCH₃ | | | 3.6280 | 0 |
| 25 | CH₃ | | CH₃ | OCH₃ | | 3.1160 | 50 |
| 26 | Br | CH₃ | CH₃ | | | | |
| 27 | | | t-Bu | | | 3.9930 | 0 |
| 28 | O—Et | Br | | | | | |
| 29 | F | | | | | 2.6450 | 60 |
| 30 | | | C₆H₅CH₃ | | | 4.7520 | 20 |
| 31 | | F | | | | 2.6840 | 85 |
| 32 | Cl | | | | Cl | 3.6740 | 50 |
| 33 | CH₃ | CH₃ | OCH₃ | | | 3.0400 | 20 |
| 34 | | | F | OCH₃ | | 2.6380 | 20 |
| 35 | | F | | | OCH₃ | 2.6380 | 50 |
| 36 | Cl | Cl | OCH₃ | | | | |
| 37 | Br | OCH₃ | | | | | |
| 38 | | Cl | OCH₃ | | | 3.0770 | 60 |
| 39 | | I | OCH₃ | | | 3.6750 | 35 |
| 40 | | | | SCH₃ | | | |
| 41 | O—Et | Cl | | | | | |
| 42 | Cl | Cl | O—Et | | | | |
| 43 | | | F | O—Et | | 2.9790 | 35 |
| 44 | CH₃ | CH₃ | O—Et | | | 3.3810 | 35 |
| 45 | | OCH₃ | Br | | | 3.2830 | 65 |
| 46 | CH₃ | Cl | Cl | | | | 80 |
| 47 | | CH₃ | O—Et | | | 3.1240 | 60 |
| 48 | | | Et | | | 3.2670 | 60 |
| 49 | | F | F | | | 2.8350 | 85 |
| 50 | CH₃ | | Cl | CH₃ | | 3.7520 | |
| 51 | Cl | | | Cl | | 3.7130 | 70 |
| 52 | OCH₃ | | | Br | | 3.2830 | 30 |
| 53 | Cl | | Cl | CH₃ | | 4.0460 | 50 |
| 54 | Cl | | OCH₃ | Cl | | 3.7040 | 70 |
| 55 | CH₃ | | CH₃ | Br | | 3.9580 | 70 |
| 56 | O—Et | | | Br | | 3.6240 | 50 |
| 57 | | Br | OCH₃ | | | 3.2830 | 60 |
| 58 | | | SCH₃ | | | 3.0860 | 15 |
| 59 | O—Et | | CH₃ | Cl | | 3.7510 | 35 |
| 60 | Cl | | O—Et | Cl | | 4.0450 | 90 |
| 61 | CH₃ | | Cl | Cl | | 4.0460 | |

### INCORPORATION BY REFERENCE

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. A compound comprising one or more functional groups that is capable of contacting or interacting with a binding pocket or a portion of a binding pocket of an AR ligand binding domain supporting or inducing the AR in an antagonized conformation, comprising any one or more of the following contacts:
(a) hydrogen bonding with Arg752 and Gln711;
(b) hydrogen bonding with a side chain amino group of Val746;
(c) hydrogen bonding with a side chain amide group of Gln783;
(d) hydrogen bonding with Asn705 and Gln783;
(e) hydrogen bonding with a side chain amine group of Asn705 of helix 3;
(f) interaction with a side chain amine group of Gln783 of helix 7;
(g) hydrogen bonding with residues Asn705 and Gln783;
(h) interaction with a carbonyl group of Gln783; and
(i) hydrophobic interaction with residues Leu707, Met745, and Met749 in a hydrophobic cleft.

2. The compound of claim 1, of formula I, 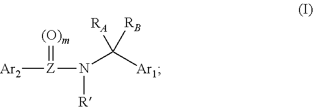
wherein,
Ar₁ is aryl or heteroaryl, each of which is optionally substituted;
Ar₂ is aryl or heteroaryl, each of which is optionally substituted;
R_{A} is H, alkyl, aralkyl, aryl, heteroaryl, halo, nitro, or CN, each of which is optionally substituted;
R_{B} is H, H, alkyl, aralkyl, aryl, heteroaryl, halo, nitro, or CN, each of which is optionally substituted;
R′ is H, alkyl, aryl, or aralkyl, each of which is optionally substituted;
Z is C or S, and
m 0, 1, or 2.

3. The compound of claim 2, wherein Ar1 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

4. The compound of claim 2, wherein Ar2 is phenyl, naphthalenyl, anthracenyl, indenyl, pyrazolyl, oxazolyl, isoxazolyl, thiazolyl, 1,2,3-, 1,2,4-, 1,2,5-, and 1,3,4-oxadiazolyl, isothiazolyl, pyridinyl, pyrazinyl, pyrimidinyl, pyridazinyl, 1,3,5-, 1,2,4-, and 1,2,3-triazinyl, benzothiofuranyl, isobenzothiofuranyl, benzisoxazolyl, benzoxazolyl, purinyl, anthranilyl, quinolinyl, isoquinolinyl, or benzoxazinyl.

5. The compound of claim 2, of formula I-A, 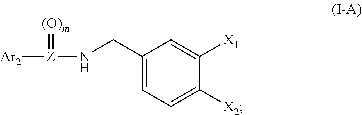
wherein,
X₁ and X₂, together with the atoms to which each is attached, form a fused carbocyclic, aryl, heterocyclic, or heteroaryl ring, each of which is optionally substituted; or
X₁ is halo or absent;
X₂ is halo or absent;
Ar₂ is aryl or heteroaryl, each of which is optionally substituted;
Z is C or S; and
m 0, 1, or 2.

6. The compound of claim 2 of formula II, 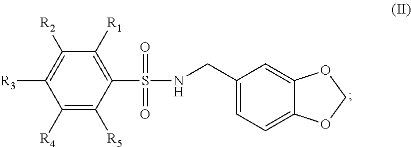
wherein,
R₁ and R₅ are each independently H, halo, alkyl, cycloalkyl, heterocycloalkyl, aryl, heteroaryl, aralkyl, alkenyl, alkynyl, haloalkyl, or alkoxy, each of which is optionally substituted;
R₂ and R₄ are each independently
(a) H, alkyl, cycloalkyl, heterocycloalkyl, aryl, heteroaryl, aralkyl, alkenyl, alkynyl, haloalkyl, or alkoxy, each of which is optionally substituted;
(b) halogen, nitro, cyano, —NC, —S—CN, or —N═C═S; or
(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
R₃ is
(a) H, alkyl, cycloalkyl, heterocycloalkyl, aryl, heteroaryl, aralkyl, alkenyl, alkynyl, haloalkyl, or alkoxy, each of which is optionally substituted;
(b) halogen, nitro, cyano, —NC, —S—CN, or —N═C═S; or
(c) C(O)R′, C(O)OR′, C(O)N(R″)(R′), OR′, OC(O)R′, OC(O)OR′, OC(O)N(R″)(R′), S(O)ₙR′, S(O)ₙC(O)R′, S(O)ₙN(R″)(R′), NR″R′, NR″C(O)R′, NR″C(O)OR′, NR″C(O)N(R″)(R′), or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and
each n is independently 0, 1, or 2.

7. The compound of claim 6, wherein
R₁ and R₅ are each independently H, halo, or optionally substituted alkyl;
R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′:
R₃ is
(a) H, alkyl, or haloalkyl; (b) halogen, nitro, or cyano; or (c) C(O)R′, C(O)OR′, OR′, NR″R′, NR″C(O)R′, or NR″S(O)ₙR′;
each R′ is independently H, alkyl, cycloalkyl, heterocycloalkyl, aryl, heteroaryl, aralkyl, alkenyl, alkynyl, haloalkyl, or alkoxy, each of which is optionally substituted;
each R″ is independently H or optionally substituted alkyl; and
each n is independently 0, 1, or 2.

8. The compound of claim 7, wherein R₃ is haloalkyl.

9. The compound of claim 7, wherein R₂ and R₄ are each independently H or haloalkyl.

10. The compound of claim 7, wherein R₃ is OR′ and R′ is haloalkyl.

11. The compound of claim 7, wherein R₂ and R₄ are each independently H or OR′ and R′ is haloalkyl.

12. The compound of claim 7, wherein R₃ is NR″R′, NR″C(O)R′, or NR″S(O)ₙR′.

13. The compound of claim 6, wherein R₃ and R₄ are each independently H, CH₃, CF₃, CN, COCH₃, COOH, COCH₂CH₃, COCH₂CH₂CH₃, CONH₂, NO₂, NC, NHCOCH₃, NHCOCH₂CH₃, NHCOCH₂Br, NHCOCH₂Cl, N(COCH₃)₂, N(COCH₂CH₃)₂, NHCOCF₃, NHSO₂CH₃, NHCOCH₂C(CH₃)₃, NCS, OCF₃, SCN, or SO₂CH₃.

14. The compound of claim 6, of formula III: 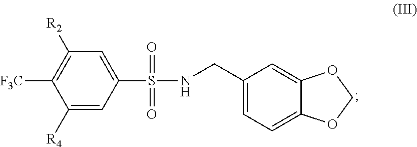
wherein R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′.

15. The compound of claim 14, wherein R₂ is H and R₄ is nitro, —NC, —S—CN, —N═C═S, or cyano.

16. The compound of claim 6, of formula IV: 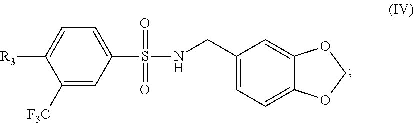
wherein R₃ is (a) H, alkyl, or haloalkyl; (b) halogen, nitro, —NC, or cyano; or (c) C(O)R′, C(O)OR′, OR′, NR″R′, NR″C(O)R′, or NR″S(O)ₙR′.

17. The compound of claim 16, wherein R₃ is nitro, —NC, cyano, C(O)R′, C(O)OR′, or NR″R′, wherein
each R′ is independently H, alkyl, or alkynyl, each of which is optionally substituted; and
each R″ is independently H or optionally substituted alkyl.

18. The compound of claim 6, of formula V: 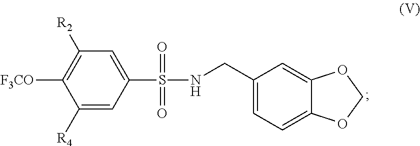
wherein R₂ and R₄ are each independently (a) H or haloalkyl; (b) halogen, nitro, cyano, —NC, —S—CN, or —N═C═S; or (c) C(O)R′, C(O)N(R″)(R′), OR′, S(O)ₙR′, NR″R′, or NR″C(O)R′;

19. The compound of claim 18, wherein R₂ is H and R₄ is C(O)R′, C(O)N(R″)(R′), or S(O)ₙR′; and n is 2; wherein
each R′ is independently H, alkyl, cycloalkyl, heterocycloalkyl, aryl, heteroaryl, aralkyl, alkenyl, alkynyl, haloalkyl, or alkoxy, each of which is optionally substituted; and
each R″ is independently H or optionally substituted alkyl.

20. The compound of claim 6, of formula VI: 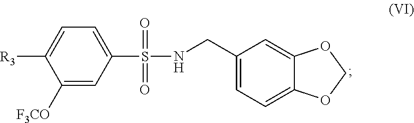
wherein R₃ is (a) H, alkyl, or haloalkyl; (b) halogen, nitro, —NC, or cyano; or (c) C(O)R′, C(O)OR′, OR′, NR″R′, NR″C(O)R′, or NR″S(O)ₙR′.

21. The compound of claim 120, wherein R₃ is nitro, C(O)R′, or C(O)OR′, wherein
each R′ is independently H, alkyl, or alkynyl, each of which is optionally substituted.

22. The compound of claim 2, selected from the following:

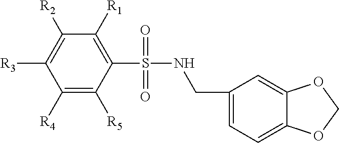

| Entry | R₁ | R₂ | R₃ | R₄ | R₅ |
|---|---|---|---|---|---|
| 1 | | —NO₂ | —CF₃ | | |
| 2 | | —NC | —CF₃ | | |
| 3 | | —SCN | —CF₃ | | |
| 4 | | —N═C═S | —CF₃ | | |
| 5 | | —CN | —CF₃ | | |
| 6 | | —CF₃ | —NO₂ | | |
| 7 | | —CF₃ | 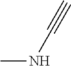 | | |
| 8 | | —CF₃ | —NC | | |
| 9 | | —CF₃ | —COOH | | |
| 10 | | —CF₃ | —C(O)CH₂CH₃ | | |
| 11 | | —CF₃ | —C(O)(CH₂)₂CH₃ | | |
| 12 | | —CF₃ | —C(O)CH₃ | | |
| 13 | | —SO₂CH₃ | —OCF₃ | | |
| 14 | | —C(O)CH₃ | —OCF₃ | | |
| 15 | | —C(O)NH₂ | —OCF₃ | | |
| 16 | | —OCF₃ | —C(O)CH₃ | | |
| 17 | | —OCF₃ | —C(O)CH₂CH₃ | | |
| 18 | | —OCF₃ | —COOH | | |
| 19 | | —OCF₃ | —NO₂ | | |
| 20 | | | —N(COCH₃)₂ | | |
| 21 | | | —NHCOCH₃ | | |
| 22 | | | —NHCOCH₂CH₃ | | |
| 23 | | | —NHCOCH₂Br | | |
| 24 | | | —NHCOCH₂Cl | | |
| 25 | | | 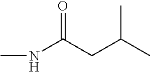 | | |
| 26 | | | —NHSO₂CH₃ | | |
| 27 | | | —NHCOCF₃ | | |
| 28 | | | —N(COCH₂CH₃)₂ | | |
| 29 | | —NHCOCH₂Cl | | | |
| 30 | | —NHCOCH₂Br | | | |
| 31 | | —SCN | | | |
| 32 | | —C(O)CH₂CH₃ | —CH₃ | | |
| 33 | | —C(O)CH₃ | —CH₃ | | |
| 34 | | —CF₃ | —CN | | |
| 35 | | —C(O)CH₃ | —NO₂ | | |
| 36 | | —C(O)CH₂CH₃ | —NO₂ | | |
| 37 | | | 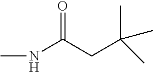 | | |
| 38 | | —C(O)CH₂CH₃ | —OCF₃ | | |

23-28. (canceled)

29. A method for modulating activity of an androgen receptor, comprising contacting an androgen receptor with a compound of claim 1.

30. The method of claim 29, wherein the androgen receptor is in a cell.

31. A method of antagonizing an androgen receptor, comprising administering an effective amount of the compound of claim 1, to a subject.

32. (canceled)

33. A method for treating a subject having a condition susceptible to treatment with an androgen receptor antagonist, comprising administering to the subject a therapeutically effective amount of a compound of claim 1.

34. The method of claim 33, wherein the condition is selected from: cancer, cancer cachexia, Kennedy's disease, adenomas and neoplasies of the prostate and malignant tumor cells expressing the androgen receptor; and osteosarcoma.

35. The method of claim 34, wherein the subject has cancer.

36. The method of claim 35, wherein the subject has androgen dependent prostate cancer.

37. The method of claim 35, wherein the subject has androgen independent prostate cancer.

38. The method of claim 35, wherein the subject has prostate cancer that has relapsed after AR deprivation therapy (ADT).

39. The method of claim 33, further comprising the step of identifying a subject having a condition susceptible to treatment with the compound of claim 1.

40. A method for decreasing the activity and amount of androgen receptor in cells, comprising contacting the cells with a compound of claim 1;
wherein the amount of androgen receptors in the cells is decreased.

41. (canceled)

42. (canceled)

43. The method of claim 42, further comprising the use of an additional therapeutic agent.

44-46. (canceled)

47. A pharmaceutical composition comprising the compound of claim 1, for use in treating a subject having a condition susceptible to treatment with an androgen receptor antagonist.

48. A method for identifying a compound which modulates the activity of AR, the method comprising: contacting AR in an antagonized conformation with a compound under conditions suitable for modulation of the activity of said AR; and detecting modulation of the activity of said AR by the compound,
wherein the compound interacts with a binding site comprising one or more of
(a) hydrogen bonding with residues Arg752 and Gln711;
(b) hydrogen bonding with a side chain amino group of Val746;
(c) hydrogen bonding with a side chain amide group of Gln783;
(d) hydrogen bonding with Asn705 and Gln783;
(e) hydrogen bonding with a side chain amine group of Asn705 of helix 3;
(f) interaction with a side chain amine of Gln783 of helix 7;
(g) hydrogen bonding with residues Asn705 and Gln783;
(h) interaction with a carbonyl group of Gln783; and
(i) hydrophobic interaction with residues Leu707, Met745, and Met749 in a hydrophobic cleft; or
A method of identifying a compound that modulates AR, comprising:
a) using a three dimensional model/structure of a binding site of an antagonized conformation of AR, wherein said binding site comprises one or more of Arg752; Gln711; Val746; Gln783; Asn705; Leu707; Met745; and Met749; and
b) employing said antagonized conformation of AR template to select said AR modulator compound, wherein said modulator compound binds to said binding site; or
A method of identifying a compound that modulates AR, the method comprising:
a. providing a three dimensional model/structure of a binding site of an antagonized conformation of AR, wherein said binding site comprises one or more of Arg752; Gln711; Val746; Gln783; Asn705; Leu707; Met745; and Met749; and
b. simulating a binding interaction between said binding site and a compound, wherein the interaction of the compound with the binding site occurs at one or more of Arg752; Gln711; Val746; Gln783; Asn705; Leu707; Met745; and Met749; and
c. determining whether said compound binds to one or more amino acid residues selected from the group consisting of, Arg752; Gln711; Val746; Gln783; Asn705; Leu707; Met745; and Met749, of said binding site, wherein said compound which binds to said amino acid residue of the binding site.

49-50. (canceled)
